(19) United States
(12) Patent Application Publication
(10) Pub. No.: US 2020/0023081 A1
(43) Pub. Date: Jan. 23, 2020

(54) SELF-ASSEMBLING CYCLOPEPTIDE-DYE COMPOUNDS, FORMULATIONS THEREOF, AND USES THEREOF

(71) Applicant: The Board of Trustees of the Leland Stanford Junior University, Stanford, CA (US)

(72) Inventors: Hao Chen, Menlo Park, CA (US); Zhen Cheng, Mountain View, CA (US)

(21) Appl. No.: 16/519,101

(22) Filed: Jul. 23, 2019

Related U.S. Application Data
(60) Provisional application No. 62/702,069, filed on Jul. 23, 2018.

(51) Int. Cl. A61K 49/00; C09B 69/10

(52) U.S. Cl. CPC A61K 49/0056; C09B 69/109

(57) ABSTRACT

Provided are pH-sensitive self-assembling cyclopeptide-dye CH-RGD molecules that sharply respond to pH changes in a diseased tissue and which are useful as NIR-II imaging agents. The pH-sensitive cyclopeptide-dye molecules in a slightly acidic environment can self-assembling or aggregate into nanoparticles with enhanced fluorescence. They combine the advantages of both small molecule and nanoparticles, while being safe, excretable, and show high tumor accumulation and retention time properties. The probes show micro-scale resolution in blood and lymphatic vessels, and high contrast and resolution in mice bone/lymph node/orthotopic/metastatic tumor imaging. They accumulate in tumors after intradermal injection and allow intradermal injection-mediated NIR-II fluorescent imaging guided orthotopic tumor surgery.

### CROSS-REFERENCE TO RELATED APPLICATIONS

This application claims priority to and the benefit of U.S. Provisional Application 62/702,069 titled “SELF-ASSEMBLING CYCLOPEPTIDE-DYE COM POUNDS, FORMULATIONS THEREOF, AND USES THEREOF” filed Jul. 23, 2018, the disclosure of which is incorporated herein by reference in its entirety.

### STATEMENT ON FUNDING PROVIDED BY THE U.S. GOVERNMENT

This invention was made with Government support under contract CA151459 awarded by the National Institutes of Health and contract DE-SC0008397 awarded by the Department of Energy. The Government has certain rights in the invention.

### TECHNICAL FIELD

The present disclosure is generally related to pH-dependent self-assembling cyclopeptide-dye nanoparticles. The present disclosure is further related to uses of the pH-dependent self-assembling cyclopeptide-dye nanoparticles for imaging of tumors.

### BACKGROUND

Nanomedicines have numerous benefits for cancer therapy drug development, demonstrating the enhanced permeability and retention (EPR) effect and specific tumor targeting abilities. However, complex formulas and ambiguous chemical formulas have stood in the way of FDA approval of nanosized drugs (Fanciullino et al., (2013) Crit. Rev. Oncol. Hematol. 88: 504-513). Furthermore, while small molecules can have properties that allow them to respond to stimuli like pH changes, designing small molecules that are responsive to changes in physiological pH conditions (approximately pH 6.8-7.4) has been difficult.

An acidic microenvironment is found in solid tumor tissues (pH 6.5-6.8) (Wang et al., 2014) Nat. Mater. 13: 204-212) and inflammatory sites (Kato et al., (2013) Cancer Cell Int. 13: 89), and is associated with bone resorption by osteoclasts (pH less than 5.5) (Baron et al., (1985) J. Cell Biol. 101: 2210-2222). A pH sensitive agent responsive to the acidic microenvironment of solid tumor tissues (pH 6.5-6.8) (Wang et al., 2014) Nat. Mater. 13: 204-212) would be invaluable for potential therapeutic drugs. Previous sharp pH-responsive nanoparticles (translation at pH 6.7, ΔpH_{ON/OFF} less than 0.25) mapped the tumor with a high contrast ratio (Wang et al. 2014) Nat. Mater. 13: 204-212) and were capable of fluorescent image guided surgery (Zhao et al., (2017) Nat. Biomed. Eng. 1: 0006), but were less clinically translatable, while potentially translatable small molecule tracers lack sharp pH responses.

Fluorescent imaging is clinically used for blood perfusion evaluation of organs transplantation after vascular anastomosis (Sekijima et al., (2004) Transplant Proc. 36: 2188-2190), sentinel lymph node dissection (Zhang et al., (2016) Plos One 11: e0155597), and imaging guided oncologic surgery (Mondal et al., (2014) Adv. Cancer Res. 124: 171-211). Fluorescent imaging in the second near-infrared window (NIR-II; 1,000-1,700 nm) has shown significantly improved imaging quality due to diminished tissue autofluorescence, reduced photon scattering, and minor light absorption at longer wavelengths, encouraging the development of small molecule dyes to replace nanoparticle fluorophores for NIR-II imaging (Antaris et al., (2016) Nat. Mater. 15, 235-242). However, small molecule dyes often suffer from low quantum yield. Several methods have been developed to improve the low quantum yield (Antaris et al., (2017) Nat. Commun. 8: 15269; Yang et al., (2017) Adv. Mater 0.29: Cheng et al., (2017) ACS Nano. 11, 12276-12291), but increase the metabolic load.

### SUMMARY

One aspect of the disclosure encompasses embodiments of a compound comprising a dye molecule attached to at least one cyclopeptide, wherein the composition can be a monomer when at a first pH and forms a self-aggregate when at a second pH, wherein the second pH is less than the first pH.

In some embodiments of this aspect of the disclosure, the at least one cyclopeptide can be conjugated directly to the dye molecule or is attached thereto via a linker.

In some embodiments of this aspect of the disclosure, the at least one at least one cyclopeptide can be according to Formula I:

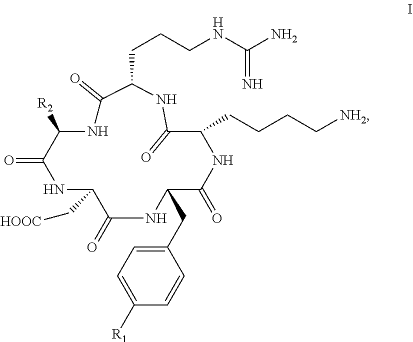

wherein R₁ can be OH or H, and R₂ can be H or CH₃.

In some embodiments of this aspect of the disclosure, the dye molecule can be a NIR-II dye.

In some embodiments of this aspect of the disclosure, the dye molecule can have a formula selected from the group consisting of:

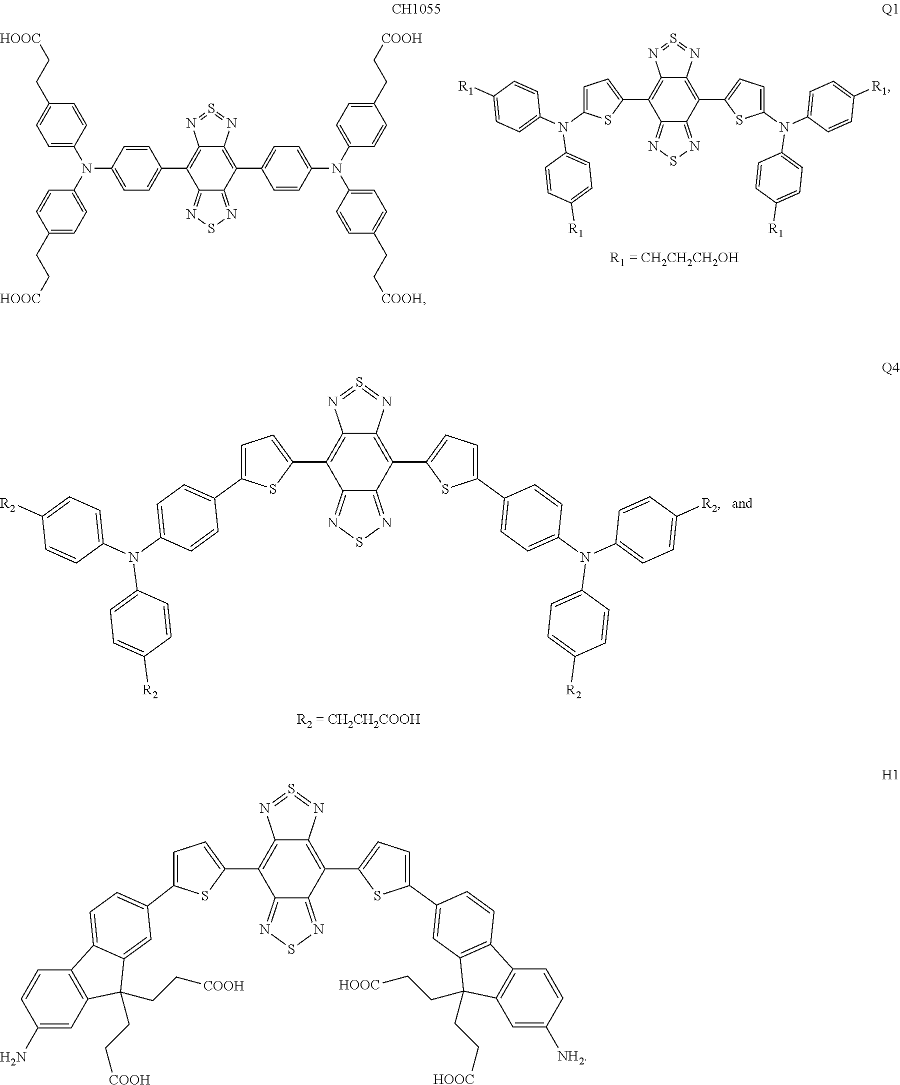

In some embodiments of this aspect of the disclosure, the cyclopeptide can be attached to the dye molecule via a polyethylene glycol linker.

In some embodiments of this aspect of the disclosure, the compound can form a self-aggregate when at a pH ranging from about 6.5 to about 7.0.

In some embodiments of this aspect of the disclosure, the compound can form a self-aggregate when at a pH lower than about 6.5.

Another aspect of the disclosure encompasses embodiments of a composition comprising: a compound comprising a dye molecule attached to at least one cyclopeptide, wherein the composition can be a monomer at a first pH and forms a self-aggregate at a second pH, wherein the second pH is less than the first pH; and a pharmaceutically acceptable carrier.

In some embodiments of this aspect of the disclosure, the at least one cyclopeptide can be conjugated directly to the dye molecule or is attached thereto via a linker.

In some embodiments of this aspect of the disclosure, the at least one cyclopeptide can be according to Formula I:

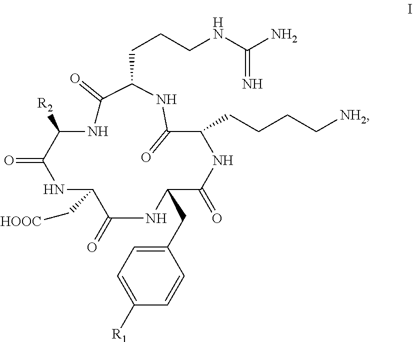

wherein R₁ can be OH or H and R₂ can be H or CH₃.

In some embodiments of this aspect of the disclosure, the dye molecule is a NIR-II dye.

In some embodiments of this aspect of the disclosure, the dye molecule has a formula selected from the group consisting of:

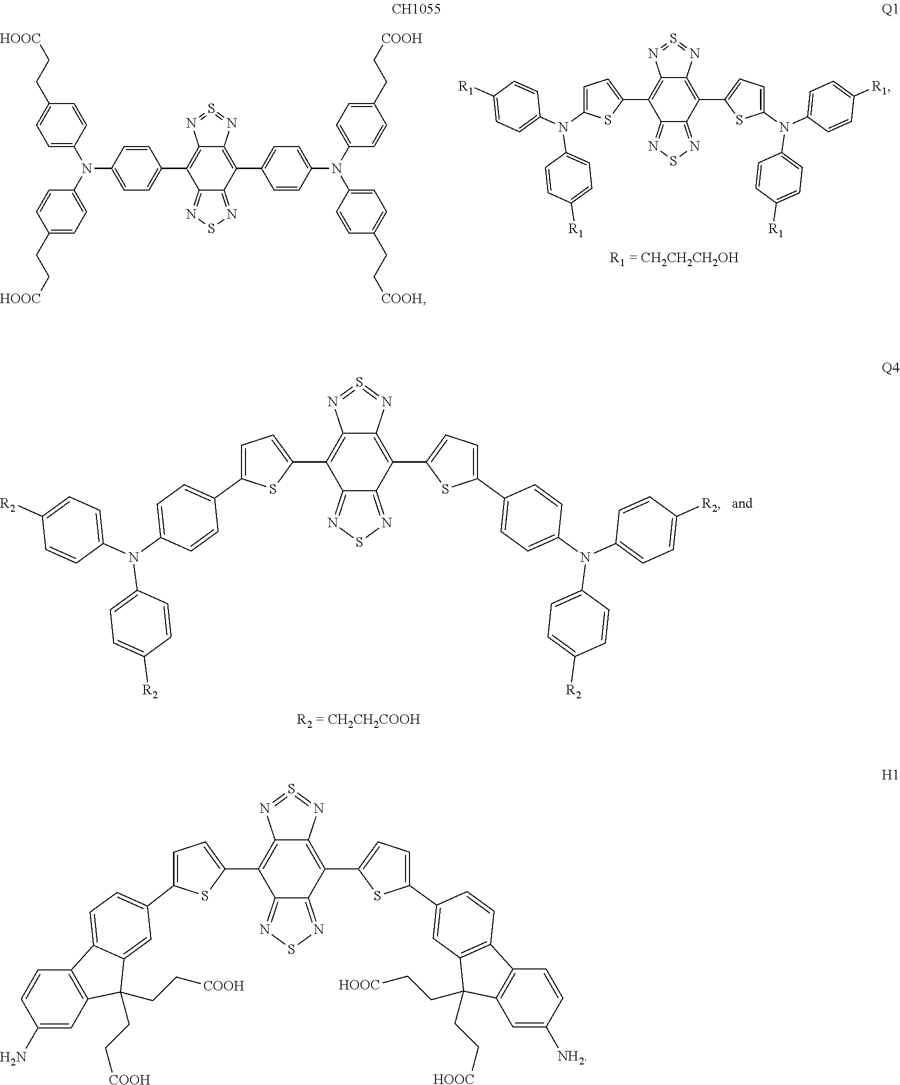

In some embodiments of this aspect of the disclosure, the at least one cyclopeptide can be attached to the dye molecule via a polyethylene glycol linker.

In some embodiments of this aspect of the disclosure, the composition can have a pH greater than the pH at which the compound forms a self-aggregate.

In some embodiments of this aspect of the disclosure, the composition can have a pH at which the compound forms a self-aggregate.

Yet another aspect of the disclosure encompasses embodiments of a method comprising: administering to an animal or human subject a composition comprising: a compound comprising a dye molecule attached to at least one cyclopeptide, wherein the composition can be a monomer at a first pH and forms a self-aggregate at a second pH, wherein the second pH can be less than the first pH; and a pharmaceutically acceptable carrier.

In some embodiments of this aspect of the disclosure, the at least one cyclopeptide is according to Formula I

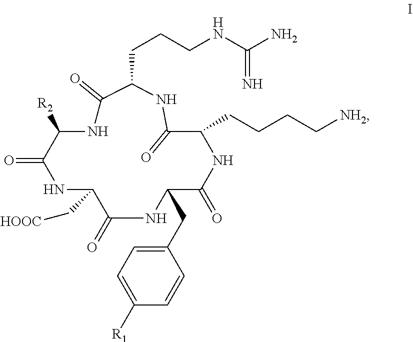

wherein R₁ can be OH or H and R₂ can be H or CH₃, and the dye molecule can have a formula selected from the group consisting of:

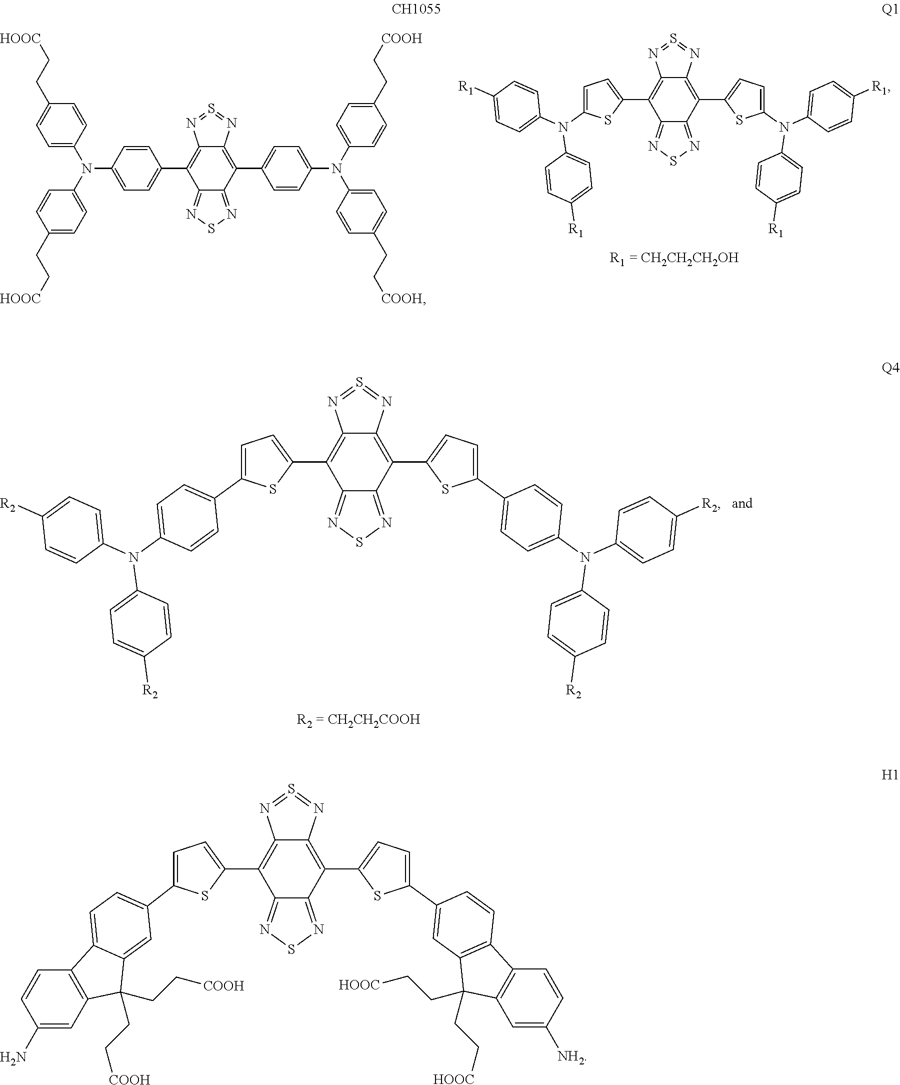

In some embodiments of this aspect of the disclosure, the method can further comprise the step of imaging a portion of the subject using a fluorescent imaging technique.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1A and 1B show pH sensitive self-assembled CH-RGD for NIR-II in vivo imaging. FIG. 1A illustrates a schematic of CH-RGD self-assembly (top), TEM images and chemical structure (bottom) in different pH buffers.

FIG. 1B illustrates a schematic of in vivo behavior of CH-RGD nanoparticles after intravenous injection (IV) or intradermal injection (ID).

FIGS. 2A-2P illustrate CH1055 derivatives self-assembly and pH sensitive properties.

FIG. 2A illustrates the synthesis of CH-RGD derivatives. Inserts: images of CH1055 (left) and CH-c(RGDyk) (right) dispersed in water.

FIG. 2B shows hydrodynamic diameters of CH-c(RGDyk), CH-3c(RGDyk) and CH-PEG2-c(RGDfk) measured by DLS.

FIGS. 2C-2E show CH1055 derivatives were dissolved in water at the concentration of 39 μM, fluorescence spectrum measurement.

FIG. 2F shows NIR-II fluorescent images (10 ms, 1000LP) of CH1055 derivatives in water at the concentration of 1 μM, excited by 808 nm laser 140 mW/cm². Inserts: the corresponding photograph of 1 μM CH1055 derivatives.

FIG. 2G shows fluorescent brightness of 1 μM CH1055 derivatives.

FIG. 2H is a plot of the integrated fluorescence spectrum of CH1055 derivatives and IR26 at five different concentrations. Linear fits were used to calculate quantum yield.

FIG. 2I shows hydrodynamic diameters of CH-c(RGDyk) (CH-RGD) in different pH phosphate buffers measured by DLS.

FIG. 2J shows NIR-II fluorescent images (10 ms, 1000LP) of 1 μM CH-RGD in different pH phosphate buffers, the upper yellow bands in pH 5.3 and 6.5 buffer tube are CH-RGD precipitates (low pH). Inserts: the corresponding photograph of CH-RGD in different pH buffers.

FIG. 2K shows fluorescence of 1 μM CH-RGD in different pH buffers.

FIG. 2L shows photostability of CH-RGD in various biological media.

FIG. 2M shows hydrodynamic diameters of self-assembly CH-RGD nanoparticles in FBS solutions in 37° C. incubator.

FIG. 2N shows macrophage cell uptake of 10 μM CH-RGD NPs. Top: NIR-II image (500 ms, 1000LP), excitation by 785 nm, 100 mW/cm² laser; bottom: bright field image.

FIG. 2O shows CH-RGD NPs imaging, bound to U87MG cells. Left: 10 μM CH-RGD NPs, right: 10 μM CH-RGD NPs with 200 μM RGD. Top: NIR-II image (1000 ms, 1000LP), bottom: bright field image.

FIG. 2P shows CH-RGD NPs 143B cell binding imaging. Left: 10 μM CH-RGD NPs in normal pH 7.4 medium, middle: 10 μM CH-RGD NPs in adjusted pH 6.5 medium, Right: 10 μM CH-RGD NPs with 200 μM RGD. White scale bar: 50 μm.

FIGS. 3A-3N show in vivo CH-RGD and CH-RAD NPs mice bone NIR-II imaging.

FIG. 3A illustrates a supine position, top CH-RGD, bottom CH-RAD 3 h post-injection. 22 nmol CH-RGD or CH-RAD was intravenously injected into 20-25 g hairless BALB/c mice (n=6, 3 males, 3 female). NIR-II imaging (200 ms, 1150LP, 808 nm 140 mW/cm² laser excitation) was taken at different time points post-injection (post-injection).

FIG. 3B illustrates a prone position, 6 h post-injection.

FIG. 3C illustrates a Left lateral position, CH-RGD 6 h post-injection.

FIG. 3D illustrates a supine position, upper CH-RGD, lower CH-RAD 24 h post-injection.

FIG. 3E illustrates a prone position, 72 h post-injection.

FIG. 3F illustrates an NIR-II fluorescent intensity changes at 72 h post-injection in different bone tissues and BAT. Right blue dashed frame, 72 h post-injection intensity change curve; left red dashed frame, zoom at the first 24 h post-injection. The results are presented as the mean+s.d. (n=6).

FIGS. 3G-3N illustrates cross-sectional fluorescence intensity profiles (black) and Gaussian fit (red) along the white-dashed bars in a, b. Corresponding serial number (1-8), names and signal to background ratio (S/B) are attached to the curve. Scale bar: 5 mm.

FIGS. 4A-4W show blood vessel, lymphatic vessel, and lymph node imaging with CH-RGD (NIR-II), ICG and IR800-RGD (NIR-I). 15 nmol CH-RGD, CH-PEG, ICG, IR800-RGD were IV injected into hairless C57BL/6 mice (FIGS. 4A-4C) or nude mice (FIGS. 4D and 4E). NIR-II (FIGS. 4A, 4B, 4D, 1000 ms, 1350LP) and NIR-I imaging (FIGS. 4C, 4E, 200 ms, 814-851BP) was taken 10 min post-injection. FIGS. 4A-4C, C57BL/6 mice brain vessel images.

FIG. 4A shows CH-RGD NIR-II image (top) and cross-sectional fluorescence intensity profiles (bottom) taken along red-dashed lines.

FIG. 4B shows CH-PEG NIR-II image (top) and cross-sectional fluorescence intensity profiles (bottom) taken along red-dashed lines.

FIG. 4C shows ICG NIR-II image (top) and cross-sectional fluorescence intensity profiles (bottom) taken along red-dashed lines.

FIGS. 4D and 4E show nude mice in supine position, left hind limb vasculature imaging of CH-RGD (FIG. 4D), IR800-RGD (FIG. 4E).

FIGS. 4F and 4G show cross-sectional fluorescence intensity profiles taken along red-dashed lines in (FIGS. 4D and 4E). Gaussian fits to the profiles are shown in red dashed curves. Vessel width measurements were taken from Gaussian curves. Lymphatic vessels and lymph node imaging: 3 nmol CH-RGD (FIGS. 4H-4J, 4Q, 4Y), ICG (FIGS. 4K and 4R) and IR800-RGD(s) ID into BALB/c mice (n=4 per probe). NIR-II (50 ms, 1100LP)/NIR-I (100 ms, 814-851BP) imaging was taken 1 h (FIGS. 4H-4J)/(FIG. 4K), 5 h (FIG. 4Q)/FIGS. 4R and 4S), 30 d (FIG. 4U) post ID, injection site (FIG. 4I).

FIG. 4M-4O show cross-sectional fluorescence intensity profiles of (FIGS. 4I-4K) correspondingly.

FIG. 4P shows BALB/c mouse in prone position. Stars indicate position of lateral iliac and subiliac lymph nodes. Black box corresponds to the imaging field of view (FIGS. 4Q-4S). After ID of CH-RGD (FIG. 4Q), ICG (FIG. 4R), IR800-RGD (FIG. 4S), NIR-II/1 imaging were taken 5 h post-injection.

FIG. 4T shows fluorescent cross-sectional intensity profile of white dashed line on lymph nodes of (FIGS. 4Q and 4R), black for CH-RGD, red for ICG. FWHM was 1.827 mm (subiliac lymph node), 2.645 mm (lateral iliac lymph node) for CH-RGD, and 2.803 mm (subiliac lymph node) for ICG after Gaussian fitting.

FIG. 4U shows CH-RGD 30 post-injection NIR-II imaging (500 ms, 1100LP).

FIG. 4V shows subiliac lymph node NIR-II fluorescent intensity and S/B change curve (n=4).

FIG. 4W shows the NIR-II (200 ms, 1100LP) fluorescent intensity of different organs 30 d post-injection of CH-RGD (n=4). Scale bar: 5 mm

FIGS. 5A-5M show subcutaneous tumor imaging with CH-RGD, CH-RAD, CH-4PEG₂₀₀₀ and IR800-RGD.

FIG. 5A is a color photograph of a nude mouse with double xenograft brain tumor (U87MG) located on the left (approximately 1 mm) and right (approximately 8 mm) shoulder. Intravenous injection of CH-RGD 10 nmol into the double tumor nude mice (n=3). NIR-II imaging (50 ms, 1000LP) at 3 days post-injection was taken at prone (FIG. 5B), right (FIG. 5C) and left (FIG. 5D) lateral decubitus.

FIG. 5E shows 19 days post-injection, CH-RGD NIR-II imaging, right lateral decubitus. Complete NIR-II imaging information can be found at FIG. 16.

FIG. 5F shows time related fluorescent intensity (solid line, left y-axis), tumor to background ratio (dish line, right y-axis) change curve of the double tumor mice.

FIG. 5G demonstrates that the best contrast images were showed after probe administration. Specifically, 5 nmol CH-RGD, CH-RGD+free RGD (20 times excess), CH-RAD, CH-4PEG₂₀₀₀, IR800-RGD were administrated into mice (n=3 per group) by IV (top) or ID (bottom). Then NIR-II (200 ms, 1000LP) or NIR-I (200 ms, 814-851BP) images were taken at different time points. The left color photograph is the representation nude mouse with subcutaneous U87MG tumor. Green arrow points to the ID injection site. Black dashed line box is the image field of view for ID injection mice.

FIG. 5H-5L show fluorescent intensity related to time for different probes (solid line, left y-axis), tumor to background ratio (dashed line, right y-axis).

FIG. 5M show the best tumor-to-normal tissue ratios obtained by IV or ID of the different probes (n=3, p less than 0.05). Dashed line demarcates the T/B ratio obtained with NIR-I targeted probe IR800-RGD. Error bars correspond to standard deviation. Scale bar: 5 mm.

FIGS. 6A-6W show orthotopic brain tumor, osteosarcoma and its metastatic tumor imaging.

FIG. 6A is a color photograph of mouse brain. Black circle denotes the glioblastoma tumor (U87MG) inoculation point.

FIGS. 6B and 6C are T2 weighted MRI images of a representative nude mouse in the coronal (6B) and sagittal (6C) planes. Arrows point to the brain tumor.

FIG. 6D shows NIR-II fluorescence (1000 ms, 1200LP) and bright field merged imaging, 4 day post-injection of 7.5 nmol CH-RGD.

FIG. 6E shows time-related fluorescent intensity (solid line, left y-axis), TBR (dish line, right y-axis) curve of (FIG. 6D) (n=3).

FIG. 6F shows brain only, bright field (FIG. 6G), NIR-II (FIG. 6H), and their merged image (FIG. 6I) of mouse brain.

FIG. 6J shows cross-sectional fluorescence intensity profiles along blue-dashed line 1 of (FIG. 6I) (n=3).

FIG. 6K shows NIR-II imaging (2000 ms, 1100LP) of a representative histological section (50 μm) near the blue-dashed line 1 of FIG. 6I. The red dots show the aggregation of CH-RGD in low pH tumor tissue. H&E staining of a representative histological section (10 μm) of (FIG. 6I) near the blue-dashed line 1 (FIG. 6L) and 2 (FIG. 6N). T: tumor tissue. FIG. 6M, 6O show zoom image of the yellow dashed line box of FIGS. 6L and 6N accordingly.

FIG. 6P is a color photograph of the orthotopic osteosarcoma mouse.

FIG. 6Q shows NIR-II imaging (200 ms, 1050LP) (3 days post-injection, n=3 per group), time related fluorescent intensity, TBR curve.

FIG. 6R shows micro-CT image of a femur osteosarcoma bearing mouse. Right femur bone was destructed (lower right corner).

FIG. 6S and 6T show T2 weighted MRI images of a representative mouse in the sagittal (s) and transverse (c) planes demonstrate the femur osteosarcoma position.

FIG. 6U is a color photograph of a representative mouse tail with metastatic osteosarcoma (n=3).

FIG. 6V illustrates a NIR-Ill/bright field merged image (left) and a NIR-II image (right, 100 ms, 1100LP) at 1, 2, and 3 days after intradermal injection of 7.5 nmol CH-RGD.

FIG. 6W shows H&E staining section (8 μm) around the blue-dashed line in (FIG. 6Y). Scale bar: 5 mm, unless noted otherwise.

FIG. 7 shows the structure of CH1055, CH1055-RGD and RGD derivatives.

FIGS. 8A-8D show CH-RGD derivatives transmission electron microscopy (TEM) image.

FIG. 8A shows different CH-RGD derivatives TEM images.

FIG. 8B shows CH-c(RGDyk) or CH-RGD NPs in different pH buffers TEM image.

FIG. 8C shows, in different pH buffers, CH-RGD NPs size curve. Sharp change in pH 7 to approximately 6.8.

FIG. 8D illustrates CH-RGD NPs stability in FBS for a 1 month period.

FIG. 9 shows a comparison of CH-RGD NPs, ICG, and IR800-RGD NIR-II fluorescence. All probes are 10 μM in 1× pH 7.4, PBS buffer. Upper: different probes under different longpass filter normalized fluorescent intensity comparison curve; bottom: NIR-II imaging of different probes under different longpass filter.

FIG. 10 shows in vivo CH-RGD and CH-RAD NPs mice bone NIR-II imaging.

FIG. 11 shows mice femur, femur bone marrow and skull NIR-II imaging. IV inject 22 nmol CH-RGD into BALB/c mice (n=3), 10 days post injection (post-injection), mouse femur, femur bone marrow (in capillary tube) and skull were taken out and NIR-II imaging (1100IP, 500 ms). Upper row: left, inject 1× pH7.4 PBS; right, inject CH-RGD NPs. Middle row: left, femur bone marrow taken from PBS injection mice femur; right, femur bone marrow taken from CH-RGD NPs injection mice femur. Bottom row: skull taken from CH-RGD NPs injection mice brain.

FIGS. 12A-12D show CH-RGD NPs ex vivo imaging. IV inject 22 nmol CH-RGD NPs into BALB/c mice (n=3). 3 days post-injection, mice were sacrificed, abdomen and thorax were opened. Organs were token out gradually, followed by NIR-II imaging (FIG. 12A, 1150lp, 200 ms). Brown adipose tissue (BAT) were found near the thorax large blood vessels (yellow star marked). H&E staining pathological analysis was used to proof the BAT tissue (arrow points out); FIG. 12B, the NIR-II imaging comparison of BAT and muscle, this BAT is between the scapulae. FIG. 12C, BAT and muscle mean NIR-II intensity comparison. FIG. 12D, H&E staining pathological analysis of the BAT between the scapulae. Black scale bar represents 200 μm.

FIG. 13 is a graph illustrating CH-RGD biodistribution of NPs after IV injection. 22 nmol CH-RGD NPs IV injected into BALB/c mice (n=3). 3 days post-injection, mice were sacrificed, organs were harvest and token the NIR-II imaging. Mean intensities were used for the biodistribution histogram.

FIGS. 14A-14B show CH-RGD NPs blood half-life measurement.

FIG. 14A shows different time point BALB/c mice (n=5) NIR-II imaging (10001p, 500 ms). Arrows indicate the blood vessel signal.

FIG. 14B shows normalized NIR-II blood fluorescent signal curve. Half-life: approximately 776 min.

FIG. 15 shows IR800-RGD intradermal injection NIR-II imaging. IR800-RGD (3 nmol) was injected intradermally into hair-removed BALB/c mouse (n=4 per probe, male, 1 years old, weight approximately 42 g) tail base. NIR-1 imaging (100 ms, 814 approximately 851 bandpass) was taken 1 h, 3 h, 5 h post-injection. Fluorescent signal appears in mice bladder from 1 h post-injection. No lymphatic vessel or lymph node was found during the imaging period.

FIG. 16 shows double subcutaneous U87MG tumor imaging.

FIG. 17 shows subcutaneous U87MG tumor imaging.

FIG. 18 shows orthotopic osteosarcoma 143B tumor imaging. The ID CH-RGD NPs, 7 days post-injection, mice were used for NIR-II fluorescent imaging guided surgery.

FIG. 19 shows CH-RGD NPs liver signal decay after IV injection. IV inject 10 nmol CH-RGD NPs into BALB/c mice (n=5). NIR-II fluorescent imaging (10001p, 200 ms) of mice in a supine position were taken 1, 2, 3, 4, 8, 12, 32, 60 days (d) post injection. Of 5 mice, 4 gave the strongest signal 2 days post-injection. One mouse gave it 1 day post-injection. Each liver signal was normalized with the 2 days post-injection intensity. GraphPad Prism 6 one phase decay analysis was used to get the half-life approximately 7.65 d.

FIGS. 20A-20F is a series of graphs showing CH-RGD NPs IV and ID injection biodistribution histograms. Three tumor bearing nude mice per group. 5 nmol CH-RGD NPs were IV injected (FIG. 20A) or ID injected (FIG. 20B) into subcutaneous U87MG tumor-bearing mice, 7 days post-injection, NIR-II signal in different organs; 5 nmol CH-RGD NPs & 100 nmol free RGD were IV injected (FIG. 20C) or ID injected (FIG. 20D) into subcutaneous U87MG tumor bearing mice, 7 days post-injection, NIR-II signal in different organs; 5 nmol CH-RGD NPs were IV injected (FIG. 20E) or ID injected (FIG. 20F) into orthotopic 143B bone tumor bearing mice, 7 days post-injection, NIR-II signal in different organs. NIR-II imaging 10001p, 200 ms exposure time.

FIG. 21 is a graph showing CH-RGD NP cytotoxicity.

FIGS. 22A-22D show cool-white LED NIR-II imaging.

FIG. 22A illustrates 300 mW/cm2, 6000K, cool-white LED light NIR-II imaging under different longpass filter (1000-1400 nm) with a multi exposure time (20-2000 ms).

FIG. 22B illustrates a cool-white LED light spectrum.

FIG. 22C illustrates Si and InGaAs detector response curves.

FIG. 22D illustrates a cool-white LED light fluorescent intensity curve.

FIGS. 23A-23D are digital images showing dissected lymph node H&E staining pathological analysis.

FIG. 23A illustrates a dissected lymph node.

FIGS. 23B-23D show the zoom-in of the labeled b, c, and d black dashed boxes of FIG. 23A; 1, tumor embolus in lymphatic vessel; 2, tumor lymphatic metastasis; 3, tumor necrosis; 4, normal lymph node tissue. Black scale bar represents 200 μm.

FIGS. 24A-24D show dissected 143B bone tumor H&E staining pathological analysis. FIG. 24A, NIR-II image during the imaging guided surgery. Dissected 143B bone tumor; FIG. 24B, zoom-in b black dashed box of FIG. 24A, T: tumor; TN: tumor necrosis. Red scale bar represents 1 mm. Black scale bar represents 200 μm.

FIG. 25A shows the MALDI-TOF spectrum of CH-c(RGDyk).

FIG. 25B shows the MALDI-TOF spectrum of CH-3c(RGDyk).

FIG. 25C shows the MALDI-TOF spectrum of CH-PEG2-c(RGDfk).

FIG. 25D shows the MALDI-TOF spectrum of CH-c(RADyk).

FIG. 25E shows the MALDI-TOF spectrum of CH-c(RGDfk).

FIGS. 26A-26B show human cancer cell growth inhibition dose-response curves of CH-RGD NPs.

FIG. 26A illustrates the cell viability of human glioblastoma U87MG cell line. IC₅₀=2.15×10⁻⁵.

FIG. 26B illustrates the cell viability of human osteosarcoma 143B cell line. I₅₀=2.013×10⁻⁵.

### DETAILED DESCRIPTION

This disclosure is not limited to particular embodiments described, and as such may, of course, vary. The terminology used herein serves the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, dimensions, frequency ranges, applications, or the like, as such can vary. It is also to be understood that the terminology used herein is for purposes of describing particular embodiments only, and is not intended to be limiting. It is also possible in the present disclosure that steps can be executed in different sequence, where this is logically possible. It is also possible that the embodiments of the present disclosure can be applied to additional embodiments involving measurements beyond the examples described herein, which are not intended to be limiting. It is furthermore possible that the embodiments of the present disclosure can be combined or integrated with other measurement techniques beyond the examples described herein, which are not intended to be limiting.

It should be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. Further, documents or references cited in this text, in a Reference List before the claims, or in the text itself; and each of these documents or references (“herein cited references”), as well as each document or reference cited in each of the herein-cited references (including any manufacturer's specifications, instructions, etc.) are hereby expressly incorporated herein by reference.

Prior to describing the various embodiments, the following definitions are provided and should be used unless otherwise indicated.

### Definitions

As used herein, “active agent” or “active ingredient” can refer to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “agent” can refer to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “amphiphilic” can refer to a molecule combining hydrophilic and lipophilic (hydrophobic) properties.

As used herein, “attached” can refer to a covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used herein, “dose,” “unit dose,” or “dosage” can refer to physically discrete units suitable for use in a subject, each unit containing a predetermined quantity of the self-assembling cyclopeptide-dye compounds and/or a formulation thereof calculated to produce the desired response or responses in association with its administration.

As used herein, “effective amount” can refer to the amount of a compound provided herein that is sufficient to effect beneficial or desired biological, emotional, medical, or clinical response of a cell, tissue, system, animal, or human. An effective amount can be administered in one or more administrations, applications, or dosages. The term cam also include within its scope amounts effective to enhance or restore to substantially normal physiological function. The “effective amount” can refer to the amount of the self-assembling cyclopeptide-dye compounds or formulation thereof described herein that can allow for imaging of a cell, tissue, organ, or other portion of a subject to which the compound or formulation thereof is administered.

The term “hydrophilic”, as used herein, refers to substances that have strongly polar groups that are readily soluble in water.

The term “hydrophobic”, as used herein, refers to substances that lack an affinity for water; tending to repel and not absorb water as well as not dissolve in or mix with water.

The term “lipophilic”, as used herein, refers to compounds having an affinity for lipids.

The term “molecular weight”, as used herein, can generally refer to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein “peptide” can refer to chains of at least 2 amino acids that are short, relative to a protein or polypeptide.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” can refer to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” can refer to any acid or base addition salt whose counter-ions are non-toxic to the subject to which they are administered in pharmaceutical doses of the salts.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, “substantially pure” can mean an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” can refer to an amount (e.g. mass, volume, dosage, concentration, and/or time period) needed to achieve one or more desired result(s). For example, a therapeutically effective amount refers to an amount needed to achieve one or more therapeutic effects.

### Abbreviations

c(RGD)fk, Cyclo(-Arg-Gly-Asp-D-Phe-Lys); c(RGD)yk, Cyclo(-Arg-Gly-Asp-D-Tyr-Lys); c(RAD)fk, Cyclo(-Arg-Ala-Asp-D-Tyr-Lys); PEG, polyethylene glycol; NIR, near infrared; ICG, indocyanine green; RGD, arginine-glycine-asparate tripeptide; RAD, arginine-alanine-asparate tripeptide; microCT, microtomography or micro computed tomography; TEM, transmission electron microscopy; NP(s), nanoparticle(s); H&E, hematoxylin and eosin; MALDI-TOF, mMatrix assisted laser desorptionionization time-of-flight mass (spectrometry): BAT, brown adipose tissue; EPR, enhanced permeability and retention; SBR, signal to background ratio; IV, intravenous; ID, intradermal

### Discussion

In cancer chemotherapy, self-assembling small molecules and pH sensitive nanoparticles are drawing increasing attention due to their potential as therapeutic agents.

However, small molecular-based NPs sharply responding to pathological pH are scarce. The present disclosure encompasses embodiments of a pH-sensitive self-assembling cyclopeptide-dye CH-RGD for NIR-II imaging. It responds, when in a slightly acidic environment (pH 7.0-6.8), by aggregating into big nanoparticles with enhanced fluorescence. It combines the advantages of both small molecules and nanoparticles, while being safe, excretable, and showing high tumor accumulation and retention time properties. This clinically translatable probe accomplishes micro-scale resolution in blood and lymphatic vessels, and high contrast and resolution in mice bone/lymph node/orthotopic/metastatic tumor imaging. It accumulates in a tumor after intradermal injection, with a ratio of approximately 15:1 for tumor to background ratio. This allowed the first intradermal injection-mediated NIR-II fluorescent imaging guided orthotopic tumor surgery with low exposure time.

Provided are embodiments of a self-assembled amphiphilic cyclopeptide-dye probe: CH-RGD. This small molecule self-assembled nanoparticle adjusts its aggregation morphology in response to environmental pH changes, accompanied by increasing fluorescence as aggregation proceeds. In physiological pH, the fluorescence-enhanced approximately 80 nm CH-RGD NPs can be used for high resolution blood, lymphatic vessel, lymph node, bone, intestine, and brown adipose tissue (BAT) imaging. The sharp morphology response from pH 7 to 6.8 (ΔpH approximately 0.2) is essential for aggregation and stacking in acidic tumor extracellular pH, resulting in increasing tumor targeting, emission and retention time. Surprisingly, tumor imaging was found to be more efficient through intradermal (ID) injection than intravenous (IV) injection.

Nanomedicines have numerous benefits for cancer therapy drug development, demonstrating the enhanced permeability and retention (EPR) effect and specific tumor targeting abilities. However, complex formulas and ambiguous chemical formulas have stood in the way of FDA approval of nanosized drugs. Furthermore, while small molecules can have properties that allow them to respond to stimuli like pH changes, designing small molecules that are sensitive enough in physiological pH conditions (about 6.8-7.4) has been difficult.

An acidic microenvironment is found in solid tumor tissues (pH 6.5 to about 6.8), inflammatory sites, and is associated with bone resorption by osteoclasts (pH less than 5.5). A pH sensitive agent, therefore, that can respond to the acidic microenvironment of solid tumor tissues (pH 6.5-6.8) would be invaluable as potential therapeutic drugs. Previous sharp pH responsive nanoparticles (translation at pH 6.7, ΔpH_{ON/OFF} less than 0.25) mapped the tumor with a high contrast ratio, were capable of fluorescent image guided surgery, but were less clinically translatable, while potentially translatable small molecule tracers lack sharp pH responses.

Fluorescent imaging is clinically used for blood perfusion evaluation of organs transplantation after vascular anastomosis, sentinel lymph node dissection, and imaging guided oncologic surgery. Fluorescent imaging in the second near-infrared window (NIR-II; 1,000-1,700 nm) has shown significantly improved imaging quality due to diminished tissue autofluorescence, reduced photon scattering, and minor light absorption at longer wavelengths, encouraging the development of small molecule dyes to replace nanoparticle fluorophores for NIR-II imaging. However, small molecule dyes often suffer from low quantum yield. Several methods have been developed to improve the low quantum yield, but increase the metabolic load.

With these deficiencies in mind, described herein are pH-sensitive self-assembling cyclopeptide-dyes that can be used for NIR-II imaging. The compounds described herein can have a dye molecule and a cyclopeptide, where the cyclopeptide can be attached to the dye molecule via the directly or indirectly by means of an intervening linker. The compounds described herein can self-assemble in to nanospheres and/or irregular aggregate precipitants at various pHs, most particularly slightly acidic pHs. The compounds, nanospheres thereof, and/or aggregates thereof can be administered to a subject. The compounds, nanospheres thereof, and/or aggregates thereof can usefully accumulate in various tissues, such as tumors, which can allow improved imaging of specific tissues in a subject. Also described are methods of using the compounds and formulations thereof described herein. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Self-Assembling Cyclopeptide-Dye Compounds and Formulations Thereof

Described herein are compounds that can be self-assembling in response to pH and formulations thereof. At some pH levels, the compounds can self-assemble to form nanospheres. At some pH levels, the compounds can self-assemble to form nanospheres greater than about 500 nm. At some pH levels the compounds can self-assemble can aggregate into irregular precipitates. In use, the compounds can be administered and self-assemble when in various pH microenvironments. The compounds can accumulate in tissue environments that have pH levels that stimulate the compounds to self-assemble in to nanospheres and/or aggregates. The compounds can include a dye molecule and a cyclopeptide, as discussed in greater detail below. The cyclopeptide can provide a probe active targeting effect and the self-assembled nanosphere, nanoparticle, and/or aggregate enhanced permeability and retention effect (EPR). Thus, where the compounds accumulate in the tissue the more dye can be present in the area for a prolonged period that can be measured using a suitable detection technique and provide for enhanced imaging techniques.

### Self-Assembling Cylcopeptide-Dye Compounds

The compounds described herein can comprise a dye molecule and a cyclopeptide, where the cyclopeptide can attached directly or indirectly via a linker to the dye molecule. The cyclopeptide can be according to Formula I

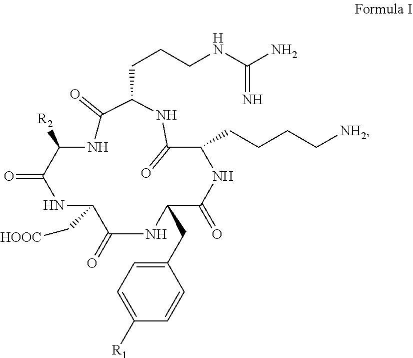

where R₁ is OH or H and R₂ is H or CH₃.

The dye molecule can be a fluorescent dye.

The dye molecule can be a near-infrared dye.

The dye molecule can be suitable for NIR-I and/or NIR-II imaging.

The dye molecule can be, but is not limited to, CH1055, Q1, Q4, and H1:

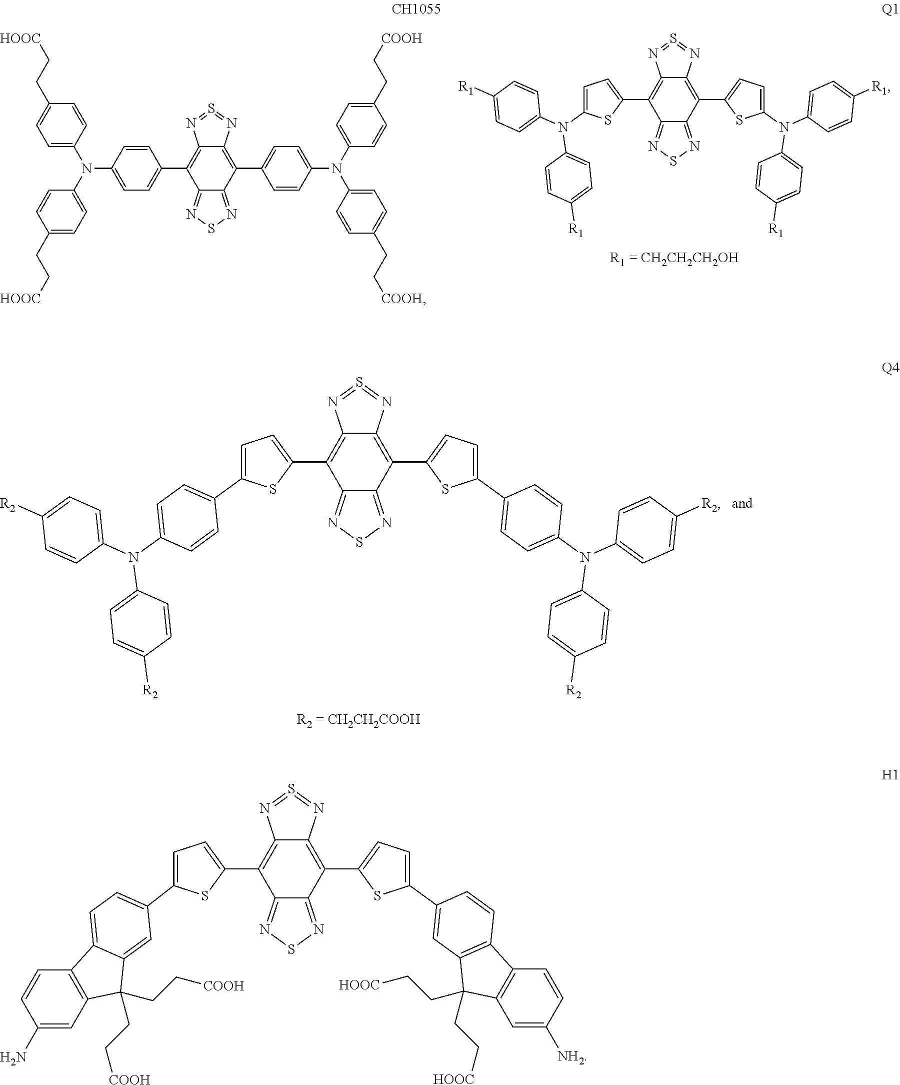

The compound can include 1, 2, 3, or 4 cyclopeptides. Each cyclopeptide can be attached directly or indirectly (e.g. via a linker) to the dye molecule at a suitable functional group on the dye. Likewise, the dye can be attached (either directly or indirectly (e.g. via a linker) to the cyclopeptide at a functional group (e.g. an amine) on the cyclopeptide. For example each cyclopeptide can be attached to a carboxyl group in CH1055. Where linkers are used to indirectly attach the cyclopeptide to the dye (e.g. CH1055), the linker can be attached to the functional group of the dye molecule, such as a carboxyl group, and a functional group, such as an amine, on the cyclopeptide.

Where a linker is present, the linker can be, but is not limited to, a polyethylene glycol (PEG), straight-chain paraffin, or branched paraffin linker. The PEG or paraffin linker (includes branched and straight chain) can have 1-10 repeat units. In some embodiments, the PEG or paraffin linker can have 2 or more repeat units.

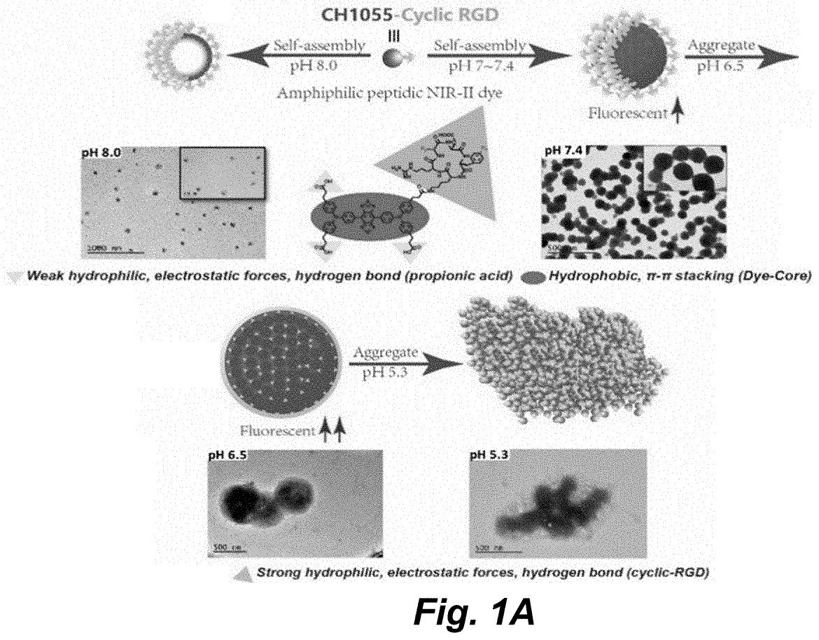

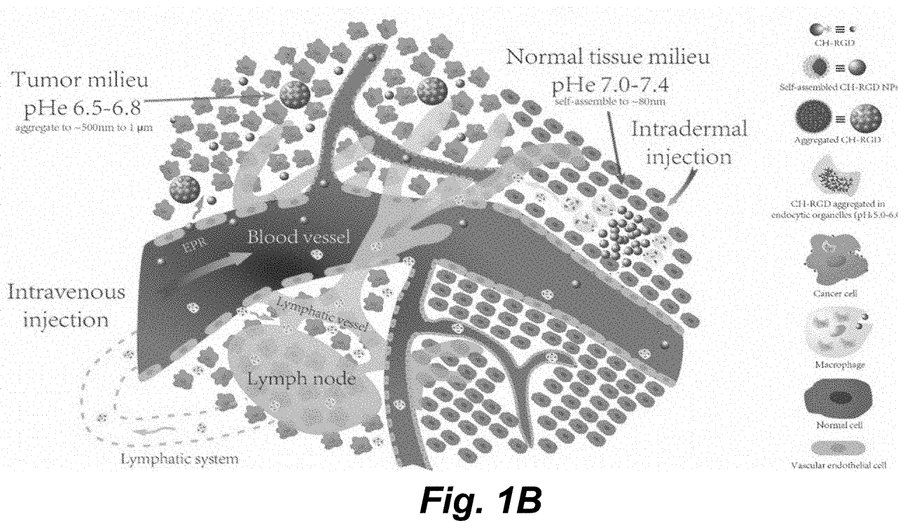

As shown in FIGS. 1A and 1B, the compounds can self-assemble into various sized nanospheres and aggregates. The size of the nanosphere or aggregate, and whether the compounds self-assemble into a nanosphere or an aggregate, can be dependent on the pH of the environment they are exposed to. The compounds can self-assemble into nanospheres at a pH ranging from about 6.5 to about 8.0. The diameter of the nanospheres can range from about 20-30 nm to 500 nm or greater. The compounds can self-assemble into particles above 500 nm in diameter at a pH ranging from about 6.5 to about 6.8. The compounds can self-assemble into aggregates of irregular precipitates at a pH lower than about 6.5. The lowest pH in physiological conditions at which these will form is above a pH 5.5. As such in some embodiments, the pH of the environment or solution in which the particles described herein can self-assemble at can range from about 5.5 to about 8.0. The particle size can be greater than 1000 nm at a pH lower than 6.5, about 1000 nm to about 500 nm at a pH range of 6.5 to 6.8 and about 80 nm at a pH range of about 7.0 to about 7.4 and about 50 nm at a pH of about 8.0.

### Formulations Containing the Self-Assembling Cyclopeptide-Dye Compounds

The self-assembling cyclopeptide-dye compounds described herein can be included in a formulation that, in addition to the compound, can further include a suitable carrier. The carrier can be a pharmaceutically acceptable carrier. The formulation can be a pharmaceutical formulation. The compounds and/or formulations described herein can be administered to a subject. The subject can be in need of diagnostic and/or therapeutic imaging of one or more regions of the body. The subject can be a subject in need thereof. The compounds and formulations described herein can be administered by a suitable route such as, but not limited to, oral, infusion, and intravenous. Other suitable routes are described elsewhere herein.

### Parenteral Formulations

The self-assembling cyclopeptide-dye compounds described herein can be formulated for parenteral delivery, such as injection or infusion, in the form of a solution or suspension. The formulation can be administered via any route, such as, the blood stream or directly to the organ or tissue to be treated.

The formulations of the disclosure may have a pH that retains the cyclopeptide-dye compounds in their non-aggregated state. However, the formulations of the disclosure may have a pH that has allowed the cyclopeptide-dye compounds to be partially or completely in their non-aggregated state. In this case, the pH of the formulation can be adjusted (elevated) to disaggregate or disassemble the nanoparticles to allow for easier administration to a recipient animal or human subject. It is contemplated, however, that suspensions of the aggregated cyclopeptide-dye compounds can also be delivered to the subject such that when the aggregates encounter the pH of the recipient tissue or serum that disassemble until they encounter a target such as a tumor.

Parenteral formulations can be prepared as aqueous compositions using techniques is known in the art. Typically, such compositions can be prepared as injectable formulations, for example, solutions or suspensions; solid forms suitable for using to prepare solutions or suspensions upon the addition of a reconstitution medium prior to injection; emulsions, such as water-in-oil (w/o) emulsions, oil-in-water (o/w) emulsions, and microemulsions thereof, liposomes, or emulsomes.

The carrier can be a solvent or dispersion medium containing, for example, water, ethanol, one or more polyols (e.g., glycerol, propylene glycol, and liquid polyethylene glycol), oils, such as vegetable oils (e.g., peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride.

Solutions and dispersions of the self-assembling cyclopeptide-dye compounds described herein can be prepared in water or another solvent or dispersing medium suitably mixed with one or more pharmaceutically acceptable excipients including, but not limited to, surfactants, dispersants, emulsifiers, pH modifying agents, and combination thereof.

Suitable surfactants can be anionic, cationic, amphoteric or nonionic surface active agents. Suitable anionic surfactants include, but are not limited to, those containing carboxylate, sulfonate and sulfate ions. Suitable anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Suitable cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Suitable nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-3-iminodipropionate, myristoamphoacetate, lauryl betaine and lauryl sulfobetaine.

The formulation can contain a preservative to prevent the growth of microorganisms. Suitable preservatives include, but are not limited to, parabens, chlorobutanol, phenol, sorbic acid, and thimerosal. The formulation can also contain an antioxidant to prevent degradation of the self-assembling cyclopeptide-dye compound(s).

The formulation can be buffered to a pH of 3-8 for parenteral administration upon reconstitution. In some aspects, the pH of the formulation can be a pH of about 7.0-7.4 upon reconstitution. Suitable buffers include, but are not limited to, phosphate buffers, acetate buffers, and citrate buffers.

Water-soluble polymers can be used in the formulations for parenteral administration. Suitable water-soluble polymers include, but are not limited to, polyvinylpyrrolidone, dextran, carboxymethylcellulose, and polyethylene glycol. Sterile injectable solutions can be prepared by incorporating the self-assembling cyclopeptide-dye compound(s) in the desired amount in the appropriate solvent or dispersion medium with one or more of the excipients listed above, as required, followed by filtered sterilization. Dispersions can be prepared by incorporating the various sterilized self-assembling cyclopeptide-dye compound(s) into a sterile vehicle which contains the basic dispersion medium and the required other ingredients from those listed above. Sterile powders for the preparation of sterile injectable solutions can be prepared by vacuum-drying and freeze-drying techniques, which yields a powder of the self-assembling cyclopeptide-dye compound(s) with or without any additional desired ingredient from a previously sterile-filtered solution thereof. The powders can be prepared in such a manner that the particles are porous in nature, which can increase dissolution of the particles. Methods for making porous particles are well known in the art.

Pharmaceutical formulations for parenteral administration can be in the form of a sterile aqueous solution or suspension of the self-assembling cyclopeptide-dye compound(s). Acceptable solvents include, for example, water, Ringer's solution, phosphate buffered saline (PBS), and isotonic sodium chloride solution. The formulation can also be a sterile solution, suspension, or emulsion in a nontoxic, parenterally acceptable diluent or solvent such as 1,3-butanediol.

In some instances, the formulation can be distributed or packaged in a liquid form. In other embodiments, formulations for parenteral administration can be packed as a solid, obtained, for example by lyophilization of a suitable liquid formulation. The solid can be reconstituted with an appropriate carrier or diluent prior to administration.

Solutions, suspensions, or emulsions for parenteral administration can be buffered with an effective amount of buffer necessary to maintain a pH suitable for ocular administration. Suitable buffers include, but are not limited to, acetate, borate, carbonate, citrate, and phosphate buffers.

Solutions, suspensions, or emulsions for parenteral administration can also contain one or more tonicity agents to adjust the isotonic range of the formulation. Suitable tonicity agents include, but are not limited to, glycerin, mannitol, sorbitol, sodium chloride, and other electrolytes.

Solutions, suspensions, or emulsions for parenteral administration can also contain one or more preservatives to prevent bacterial contamination of the ophthalmic preparations. Suitable preservatives include, but are not limited to, polyhexamethylenebiguanidine (PHMB), benzalkonium chloride (BAK), stabilized oxychloro complexes (otherwise known as Purite®), phenylmercuric acetate, chlorobutanol, sorbic acid, chlorhexidine, benzyl alcohol, parabens, thimerosal, and mixtures thereof.

Solutions, suspensions, or emulsions, use of nanotechnology including nanoformulations for parenteral administration can also contain one or more excipients, such as dispersing agents, wetting agents, and suspending agents.

### Topical Formulations

The self-assembling cyclopeptide-dye compound(s) can be formulated for topical administration. Suitable dosage forms for topical administration include creams, ointments, salves, sprays, gels, lotions, emulsions, liquids, and transdermal patches. The formulation can be formulated for transmucosal, transepithelial, transendothelial, or transdermal administration. The topical formulations can contain one or more chemical penetration enhancers, membrane permeability agents, membrane transport agents, emollients, surfactants, stabilizers, and combination thereof.

In some embodiments, the self-assembling cyclopeptide-dye compound(s) can be administered as a liquid formulation, such as a solution or suspension, a semi-solid formulation, such as a lotion or ointment, or a solid formulation. In some embodiments, the self-assembling cyclopeptide-dye compound(s) can be formulated as liquids, including solutions and suspensions, such as eye drops or as a semi-solid formulation, such as ointment or lotion for topical application to the skin, to the mucosa, such as the eye, to the vagina, or to the rectum.

The formulation can contain one or more excipients, such as emollients, surfactants, emulsifiers, penetration enhancers, and the like.

Suitable emollients include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In some embodiments, the emollients can be ethylhexylstearate and ethylhexyl palmitate.

Suitable surfactants include, but are not limited to, emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In some embodiments, the surfactant can be stearyl alcohol.

Suitable emulsifiers include, but are not limited to, acacia, metallic soaps, certain animal and vegetable oils, and various polar compounds, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In some embodiments, the emulsifier can be glycerol stearate.

Suitable classes of penetration enhancers include, but are not limited to, fatty alcohols, fatty acid esters, fatty acids, fatty alcohol ethers, amino acids, phospholipids, lecithins, cholate salts, enzymes, amines and amides, complexing agents (liposomes, cyclodextrins, modified celluloses, and diimides), macrocyclics, such as macrocylic lactones, ketones, and anhydrides and cyclic ureas, surfactants, N-methyl pyrrolidones and derivatives thereof, DMSO and related compounds, ionic compounds, azone and related compounds, and solvents, such as alcohols, ketones, amides, polyols (e.g., glycols).

Suitable emulsions include, but are not limited to, oil-in-water and water-in-oil emulsions. Either or both phases of the emulsions can include a surfactant, an emulsifying agent, and/or a liquid non-volatile non-aqueous material. In some embodiments, the surfactant can be a non-ionic surfactant. In other embodiments, the emulsifying agent is an emulsifying wax. In further embodiments, the liquid non-volatile non-aqueous material is a glycol. In some embodiments, the glycol is propylene glycol. The oil phase can contain other suitable oily pharmaceutically acceptable excipients. Suitable oily pharmaceutically acceptable excipients include, but are not limited to, hydroxylated castor oil or sesame oil can be used in the oil phase as surfactants or emulsifiers.

Lotions containing the self-assembling cyclopeptide-dye compound(s) are also described herein. In some embodiments, the lotion can be in the form of an emulsion having a viscosity of between 100 and 1000 centistokes. The fluidity of lotions can permit rapid and uniform application over a wide surface area. Lotions can be formulated to dry on the skin leaving a thin coat of their medicinal components on the skin's surface.

Creams containing the self-assembling cyclopeptide-dye compound(s) are also described herein. The cream can contain emulsifying agents and/or other stabilizing agents. In some embodiments, the cream is in the form of a cream having a viscosity of greater than 1000 centistokes, typically in the range of 20,000-50,000 centistokes. Creams, as compared to ointments, can be easier to spread and easier to remove.

One difference between a cream and a lotion is the viscosity, which is dependent on the amount/use of various oils and the percentage of water used to prepare the formulations. Creams can be thicker than lotions, can have various uses, and can have more varied oils/butters, depending upon the desired effect upon the skin. In some embodiments of a cream formulation, the water-base percentage can be about 60% to about 75% and the oil-base can be about 20% to about 30% of the total, with the other percentages being the emulsifier agent, preservatives and additives for a total of 100%.

Ointments containing the self-assembling cyclopeptide-dye compound(s) and a suitable ointment base are also provided. Suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

Also described herein are gels containing the self-assembling cyclopeptide-dye compound(s), a gelling agent, and a liquid vehicle. Suitable gelling agents include, but are not limited to, modified celluloses, such as hydroxypropyl cellulose and hydroxyethyl cellulose; carbopol homopolymers and copolymers; thermoreversible gels and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents can be selected for their ability to dissolve the drug. Other additives, which can improve the skin feel and/or emolliency of the formulation, can also be incorporated. Such additives include, but are not limited, isopropyl myristate, ethyl acetate, C₁₂-C₁₅ alkyl benzoates, mineral oil, squalene, cyclomethicone, capric/caprylic triglycerides, and combinations thereof.

Buffers can be used to control pH of a composition. The buffers can buffer the composition from a pH of about 4 to a pH of about 7.5, from a pH of about 4 to a pH of about 7, or from a pH of about 5 to a pH of about 7. In some embodiments, the buffer can be triethanolamine.

Preservatives can be included to prevent the growth of fungi and microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, and thimerosal.

In certain embodiments, the formulations can be provided via continuous delivery of one or more formulations to a patient in need thereof. For topical applications, repeated application can be done or a patch can be used to provide continuous administration of the noscapine analogs over an extended period of time.

### Enteral Formulations

The self-assembling cyclopeptide-dye compound(s) can be prepared in enteral formulations, such as for oral administration. Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Formulations containing the self-assembling cyclopeptide-dye compound(s) can be prepared using pharmaceutically acceptable carriers. As generally used herein “carrier” includes, but is not limited to, diluents, preservatives, binders, lubricants, disintegrators, swelling agents, fillers, stabilizers, and combinations thereof. Polymers used in the dosage form include, but are not limited to, suitable hydrophobic or hydrophilic polymers and suitable pH dependent or independent polymers. Suitable hydrophobic and hydrophilic polymers include, but are not limited to, hydroxypropyl methylcellulose, hydroxypropyl cellulose, hydroxyethyl cellulose, carboxy methylcellulose, polyethylene glycol, ethylcellulose, microcrystalline cellulose, polyvinyl pyrrolidone, polyvinyl alcohol, polyvinyl acetate, and ion exchange resins. “Carrier” also includes all components of the coating composition which can include plasticizers, pigments, colorants, stabilizing agents, and glidants.

Formulations containing the self-assembling cyclopeptide-dye compound(s) can be prepared using one or more pharmaceutically acceptable excipients, including diluents, preservatives, binders, lubricants, disintegrators, swelling agents, fillers, stabilizers, and combinations thereof.

Delayed release dosage formulations containing the self-assembling cyclopeptide-dye compound(s) can be prepared as described in standard references such as “Pharmaceutical dosage form tablets”, eds. Liberman et al., (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

The formulations containing the self-assembling cyclopeptide-dye compound(s) can be coated with a suitable coating material, for example, to delay release once the particles have passed through the acidic environment of the stomach. Suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings can be formed with a different ratio of water soluble polymer, water insoluble polymers and/or pH dependent polymers, with or without water insoluble/water soluble non polymeric excipient, to produce the desired release profile. The coating can be performed on a dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Additionally, the coating material can contain conventional carriers such as plasticizers, pigments, colorants, glidants, stabilization agents, pore formers and surfactants. Optional pharmaceutically acceptable excipients include, but are not limited to, diluents, binders, lubricants, disintegrants, colorants, stabilizers, and surfactants.

Diluents, also referred to as “fillers,” can be used to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. The usual diluents include inert powdered substances such as starches, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful.

Binders can impart cohesive qualities to a solid dosage formulation, and thus can ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone. Typical tablet binders include substances such as starch, gelatin and sugars such as lactose, fructose, and glucose. Natural and synthetic gums, including acacia, alginates, methylcellulose, and polyvinylpyrrolidone can also be used. Polyethylene glycol, hydrophilic polymers, ethylcellulose and waxes can also serve as binders.

Lubricants can be included to facilitate tablet manufacture. Suitable lubricants include, but are not limited to, magnesium stearate, calcium stearate, stearic acid, glycerol behenate, polyethylene glycol, talc, and mineral oil. A lubricant can be included in a tablet formulation to prevent the tablet and punches from sticking in the die. The lubricant can be chosen from such slippery solids as talc, magnesium and calcium stearate, stearic acid and hydrogenated vegetable oils.

Disintegrants can be used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone® XL from GAF Chemical Corp).

Stabilizers can be used to inhibit or retard drug decomposition reactions which include, by way of example, oxidative reactions. Suitable stabilizers include, but are not limited to, antioxidants, butylated hydroxytoluene (BHT); ascorbic acid, its salts and esters; Vitamin E, tocopherol and its salts; sulfites such as sodium metabisulphite; cysteine and its derivatives; citric acid; propyl gallate, and butylated hydroxyanisole (BHA).

### Methods of Using the Self-Assembling Cyclopeptide-Dye Compounds and Formulations Thereof

In use, the compounds and formulations thereof can be administered to a subject. The compounds once administered can circulate through the subject and self-assemble when in specific pH microenvironments that stimulate self-assembly, such as those that are slightly acidic. The compounds can accumulate in tissue environments that have pH levels that stimulate the compounds to self-assemble in to nanospheres and/or aggregates. Thus, the dye accumulates in areas where the compounds have self-assembled and accumulated and facilitates imaging areas that have pH microenvironments that stimulate self-assembly of the compounds. Tumors, for example, have an acidic environment. Thus, the compounds described herein and formulations thereof can be useful for imaging tumors. The compounds and formulations thereof described herein can be useful for use during imaging-guided surgical or other medical procedures. Such procedures include, but are not limited to, tissue resections, which can include tumor resection surgeries. The compounds and formulations thereof described herein can also be useful for imaging vasculature and related components including, but not limited to, blood and lymphatic vessels and lymph nodes. The compounds and formulations thereof described herein can be useful for vascular, lymphatic, tumor, and orthotopic imaging.

In some embodiments, an amount of the self-assembling cyclopeptide-dye compound(s) or formulation thereof can be administered to a subject having or is suspected of having a cancer or a tumor. The subject can have a condition afflicting the blood vessels. The subject can have or be suspected of having a blood vessel anastomosis. The subject can be in need of a blood vessel evaluation, such as a blood vessel anastomosis evaluation. The subject can be in need of a lymph node biopsy. The amount can be an amount sufficient to allow for imaging at least a portion of the subject. After administration, at least a portion of the subject can be imaged using a suitable imaging technique to detect the dye component of the self-assembling cyclopeptide-dye compound. In some embodiments, the imaging technique is a fluorescent imaging technique. In some embodiments, the imaging technique is a near-infrared (NIR) imaging technique. In some embodiments, the imaging technique is a NIR-I or NIR-II imaging technique.

The self-assembling cyclopeptide-dye compound(s) or formulation thereof described herein can be co-administered or be a co-therapy with another active agent or ingredient that can be included in the formulation or provided in a dosage form separate from the self-assembling cyclopeptide-dye compound(s) or formulation thereof.

The amount of the self-assembling cyclopeptide-dye compound(s) or formulation thereof can range from about 0.1 μg/kg to up to about 1000 mg/kg or more, depending on the factors mentioned elsewhere herein. In certain embodiments, the amount can range from 0.1 μg/kg up to about 500 mg/kg, or 1 μg/kg up to about 500 mg/kg, 5 μg/kg up to about 500 mg/kg, 0.1 μg/kg up to about 100 mg/kg, or 1 μg/kg up to about 100 mg/kg, 5 μg/kg up to about 100 mg/kg.

Administration of the self-assembling cyclopeptide-dye compound(s) or formulation thereof can be systemic or localized. The self-assembling cyclopeptide-dye compound(s) or formulation thereof can be administered to the subject in need thereof one or more times per hour or day. In embodiments, the self-assembling cyclopeptide-dye compound(s) or formulation thereof can be administered once daily. In other embodiments, the self-assembling cyclopeptide-dye compound(s) or formulation thereof can be administered can be administered 2, 3, 4, 5, 6, 7, 8, 9, 10, 11, 12, or more times daily. In some embodiments, when administered, an effective amount of the self-assembling cyclopeptide-dye compound(s) or formulation thereof can be administered to the subject in need thereof. The self-assembling cyclopeptide-dye compound(s) or formulation thereof can be administered one or more times per week. In some embodiments, self-assembling cyclopeptide-dye compound(s) or formulation thereof can be administered 1, 2, 3, 4, 5, 6, or 7 days per week. In some embodiments, the self-assembling cyclopeptide-dye compound(s) or formulation thereof can be administered 1, 2, 3, 4, 5, 6, 7, 8, 9, 10, 11, 12, or more times per month. In some embodiments, the self-assembling cyclopeptide-dye compound(s) or formulation thereof can be administered 1, 2, 3, 4, 5, 6, 7, 8, 9, 10, 11, 12, 13, 14, 15, 16, 17, 18, or more time per year.

In some embodiments, the self-assembling cyclopeptide-dye compound(s) or formulation thereof can be administered in a dosage form. The amount or effective amount of the self-assembling cyclopeptide-dye compound(s) or formulation thereof can be divided into multiple dosage forms. For example, the effective amount can be split into two dosage forms and the one dosage forms can be administered, for example, in the morning, and the second dosage form can be administered in the evening. Although the effective amount can be given over two or more doses, in one day, the subject can receives the effective amount when the total amount administered across all the doses is considered. The dosages can range from about 0.1 μg/kg to up to about 1000 mg/kg or more, depending on the factors mentioned above. In certain embodiments, the dosage can range from 0.1 μg/kg up to about 500 mg/kg, or 1 μg/kg up to about 500 mg/kg, 5 μg/kg up to about 500 mg/kg, 0.1 μg/kg up to about 100 mg/kg, or 1 μg/kg up to about 100 mg/kg, 5 μg/kg up to about 100 mg/kg.

CH-RGD NPs address many of the issues concerning both nanomedicines and fluorescent probes for image guided surgery. CH-RGD's functional peptide conjugations prevent excess self-aggregation while preserving the pH-sensitive, self-assembled properties, addressing many barriers that nanomedicines have faced in seeking FDA approval. The pH-sensitive properties of CH-RGD allow it to modify its aggregation morphology when presented with the pathological, low pH tumor environment. CH-RGD transitions from an approximately 80 nm NP to a larger approximately 500 nm aggregate at pH 6.5-6.8, enhancing tumor accumulation. Tumor accumulation and retention is further supplemented by RGD binding of αᵥβ₃ integrin and the EPR effect. CH-RGD's NIR-II properties allow for deeper, higher quality tissue imaging while addressing the issue of traditionally low NIR-II signals through high tumor accumulation and quantum yield. The surprising demonstrated viability of ID injection enabled successful imaging of lymphatic systems, mediated by macrophage uptake of CH-RGD, which led to further increased tumor accumulation. The CH-RGD NIR-II setup allows for live fluorescent image guided surgery without the complicated, prohibitive setups required for NIR-I image guided surgery. Additionally, the antiangiogenic effects of RGD-αᵥβ₃ integrin binding presents possible theranostic benefits of CH-RGD which will be evaluated in the future. These in vivo imaging results hold potential for a clinically translatable, responsive probe for cancer therapy imaging.

One aspect of the disclosure encompasses embodiments of a compound comprising a dye molecule attached to at least one cyclopeptide, wherein the composition can be a monomer when at a first pH and forms a self-aggregate when at a second pH, wherein the second pH is less than the first pH.

In some embodiments of this aspect of the disclosure, the at least one cyclopeptide can be conjugated directly to the dye molecule or is attached thereto via a linker.

In some embodiments of this aspect of the disclosure, the at least one at least one cyclopeptide can be according to Formula I:

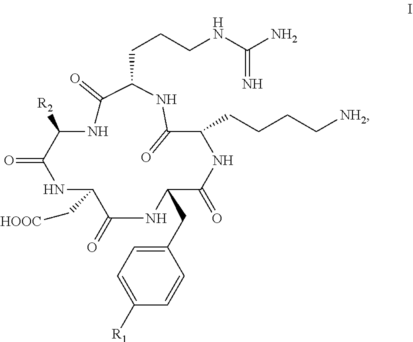

wherein R₁ can be OH or H, and R₂ can be H or CH₃.

In some embodiments of this aspect of the disclosure, the dye molecule can be a NIR-II dye.

In some embodiments of this aspect of the disclosure, the dye molecule can have a formula selected from the group consisting of:

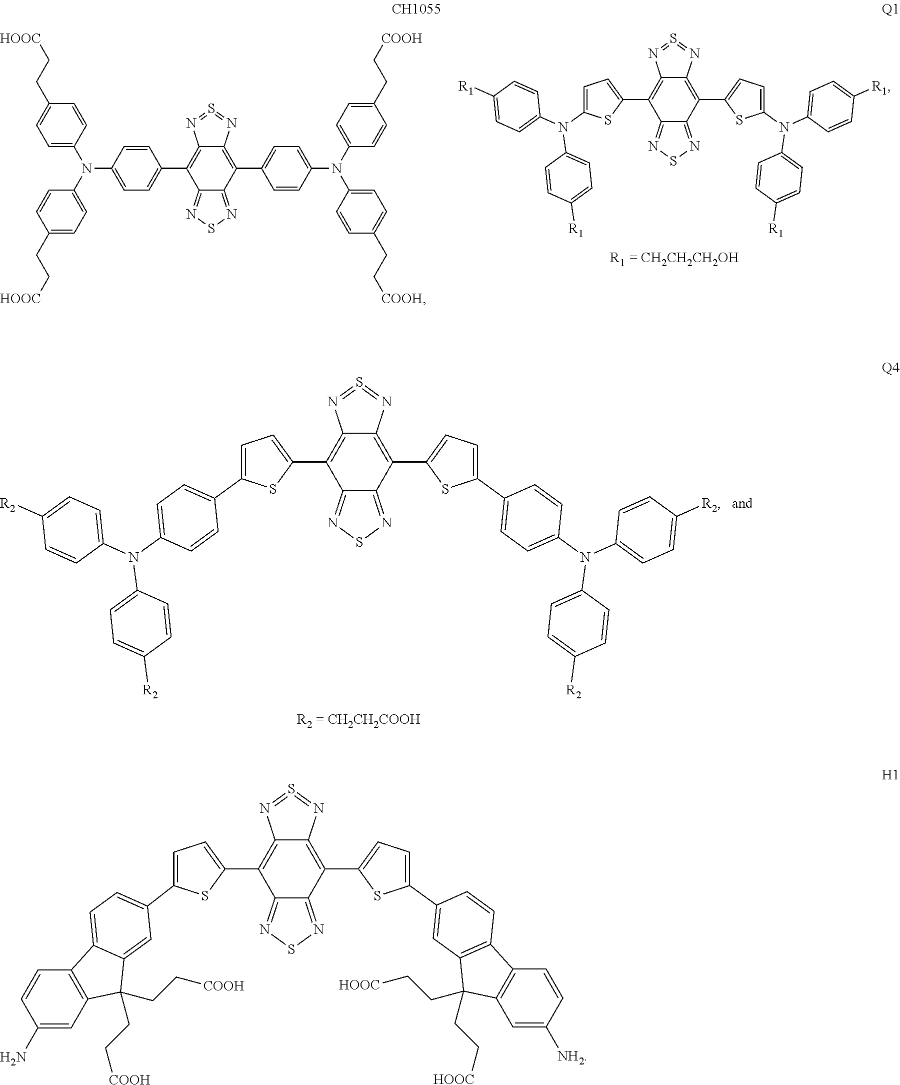

In some embodiments of this aspect of the disclosure, the cyclopeptide can be attached to the dye molecule via a polyethylene glycol linker.

In some embodiments of this aspect of the disclosure, the compound can form a self-aggregate when at a pH ranging from about 6.5 to about 7.0.

In some embodiments of this aspect of the disclosure, the compound can form a self-aggregate when at a pH lower than about 6.5.

Another aspect of the disclosure encompasses embodiments of a composition comprising: a compound comprising a dye molecule attached to at least one cyclopeptide, wherein the composition can be a monomer at a first pH and forms a self-aggregate at a second pH, wherein the second pH is less than the first pH; and a pharmaceutically acceptable carrier.

In some embodiments of this aspect of the disclosure, the at least one cyclopeptide can be conjugated directly to the dye molecule or is attached thereto via a linker.

In some embodiments of this aspect of the disclosure, the at least one cyclopeptide can be according to Formula I:

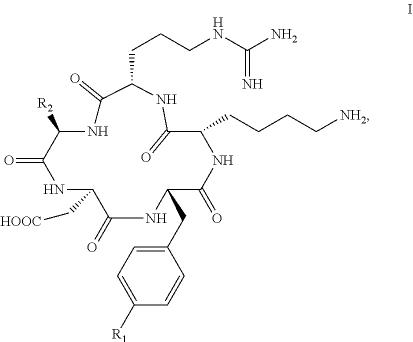

wherein R₁ can be OH or H and R₂ can be H or CH₃.

In some embodiments of this aspect of the disclosure, the dye molecule is a NIR-II dye.

In some embodiments of this aspect of the disclosure, the dye molecule has a formula selected from the group consisting of:

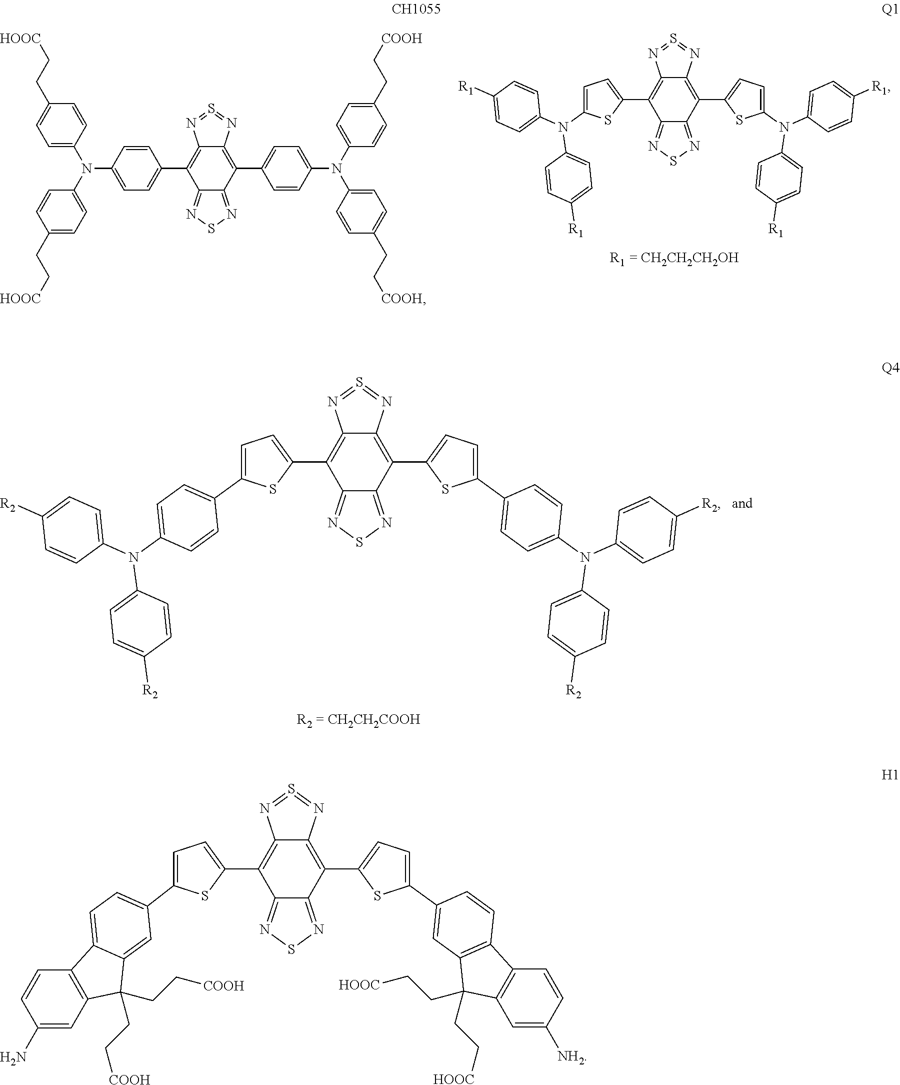

In some embodiments of this aspect of the disclosure, the at least one cyclopeptide can be attached to the dye molecule via a polyethylene glycol linker.

In some embodiments of this aspect of the disclosure, the composition can have a pH greater than the pH at which the compound forms a self-aggregate.

In some embodiments of this aspect of the disclosure, the composition can have a pH at which the compound forms a self-aggregate.

Yet another aspect of the disclosure encompasses embodiments of a method comprising: administering to an animal or human subject a composition comprising: a compound comprising a dye molecule attached to at least one cyclopeptide, wherein the composition can be a monomer at a first pH and forms a self-aggregate at a second pH, wherein the second pH can be less than the first pH; and a pharmaceutically acceptable carrier.

In some embodiments of this aspect of the disclosure, the at least one cyclopeptide is according to Formula I

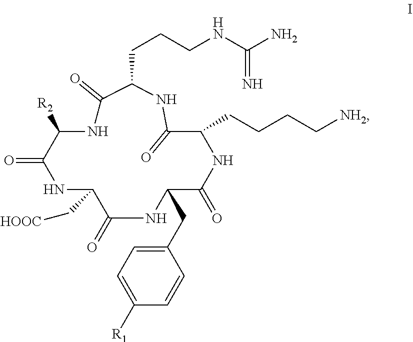

wherein R₁ can be OH or H and R₂ can be H or CH₃, and the dye molecule can have a formula selected from the group consisting of:

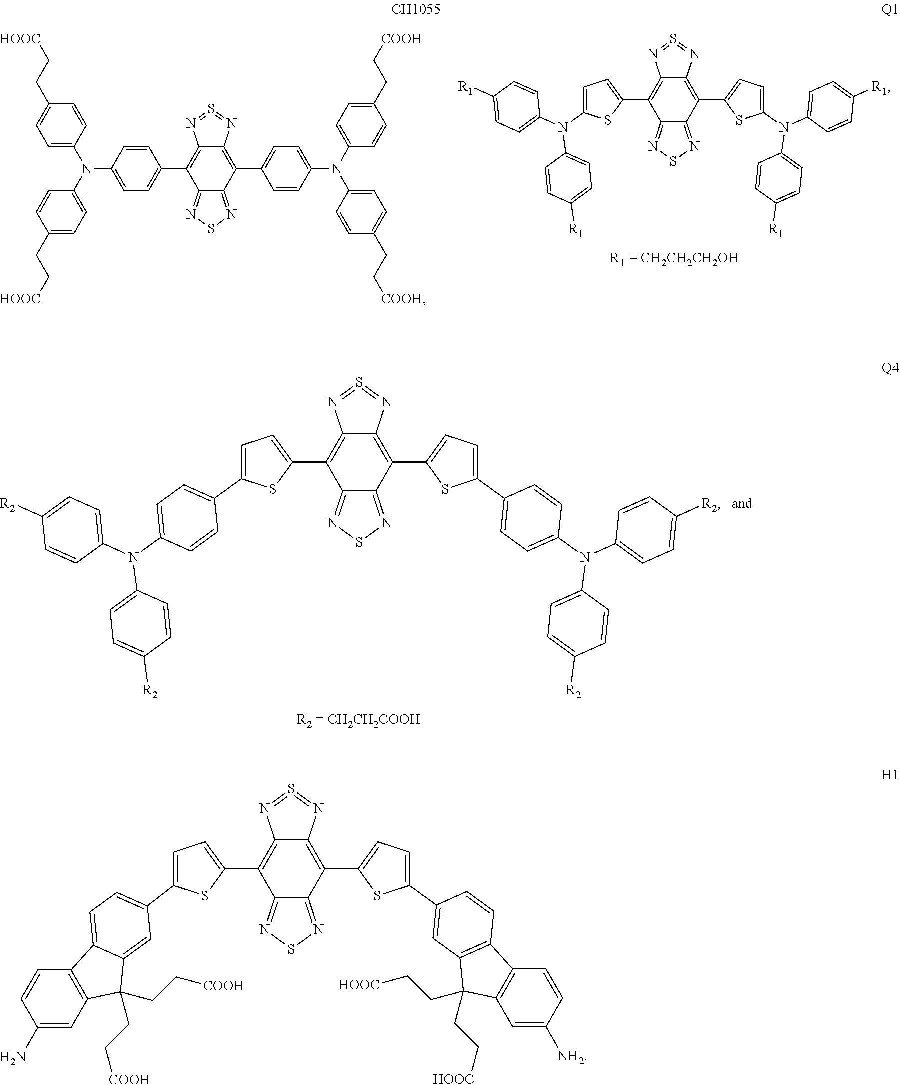

In some embodiments of this aspect of the disclosure, the method can further comprise the step of imaging a portion of the subject using a fluorescent imaging technique.

Now having described the embodiments of the present disclosure, in general, the following examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### EXAMPLES

### Example 1

### Design, Synthesis and Optical Characterization

RGD is an extensively studied self-assembly building block (Cui et al., (2010) Biopolymers 94: 1-18). The hydrophilic carboxy and guanidine groups of RGD can protonate and deprotonate in response to changes in pH, inducing changes in the peptide's noncovalent bond interaction, leading to a conformational and morphological changes to the whole nanoparticle. This gives RGD-peptide based amphiphilic materials pH-sensitive self-assembly properties (Jin et al., (2008) Macromol. Rapid Comm. 29: 1726-1731).

The traditional amphiphilic cyclic RGD/aryl structure was changed by adding weakly hydrophilic propionic acid side chains onto the hydrophobic dye-core (FIG. 1A) to induce pH-sensitive self-assembly properties. The pH induced morphology changes occur when the free carboxylic acid groups of the dye-core overcome π-π stacking interactions. The cyclopeptide composition and conjugation number were controlled to gradually modify the probe's water solubility and isoelectric point. This allows for systematic control over the self-assembly size and pH response point of the probe. The cyclo-RGD and RAD mono-substituted CH1055 (Antaris et al., (2016) Nat. Mater. 15, 235-242) were selected for having sharp self-assembly response at pH 6.8-7.0, taking on different morphologies in normal and tumor tissue milieu (FIG. 1A), as well as showing increased fluorescence for NIR-II in vivo imaging (FIG. 1B).

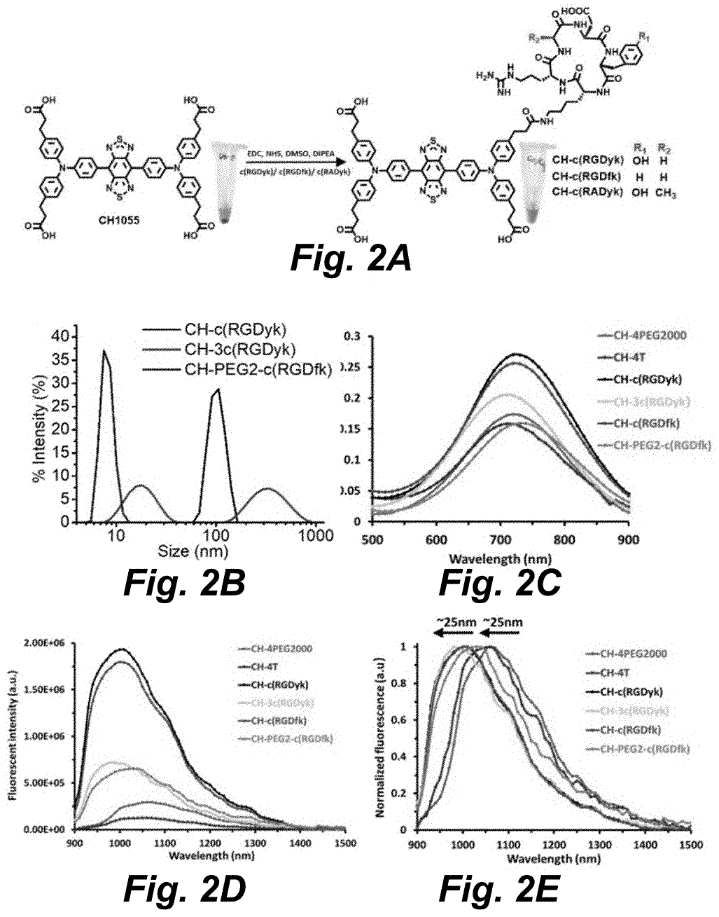

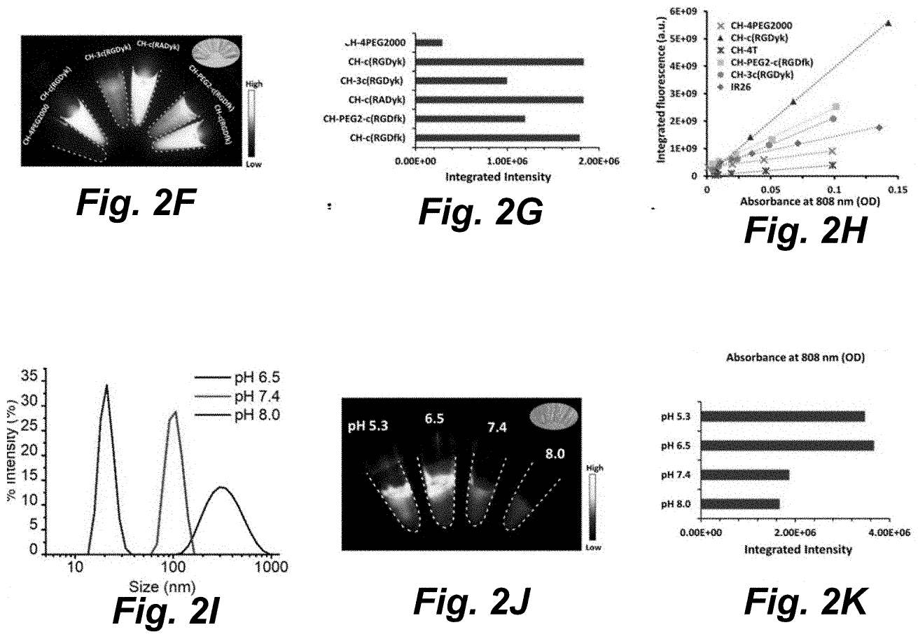

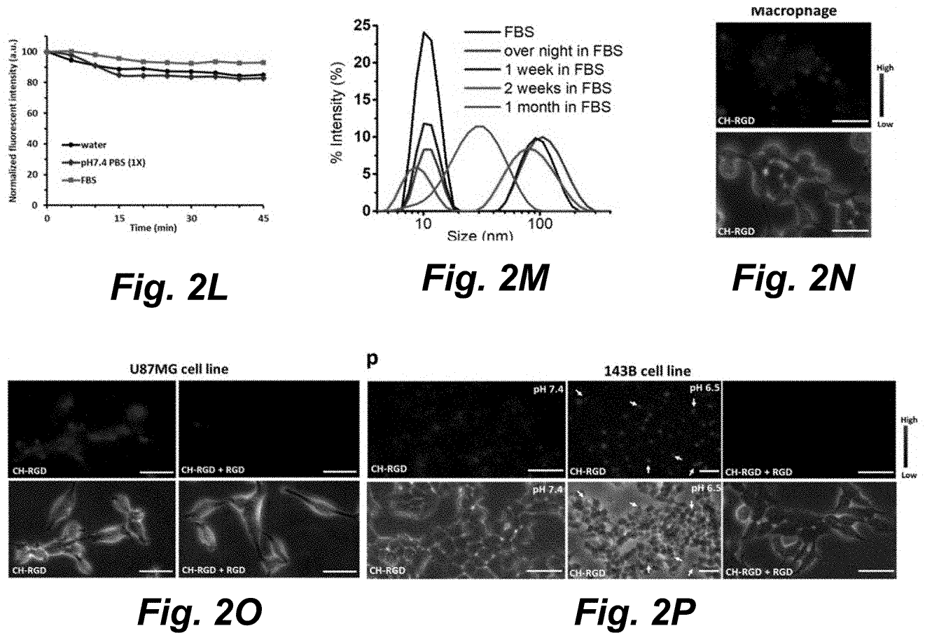

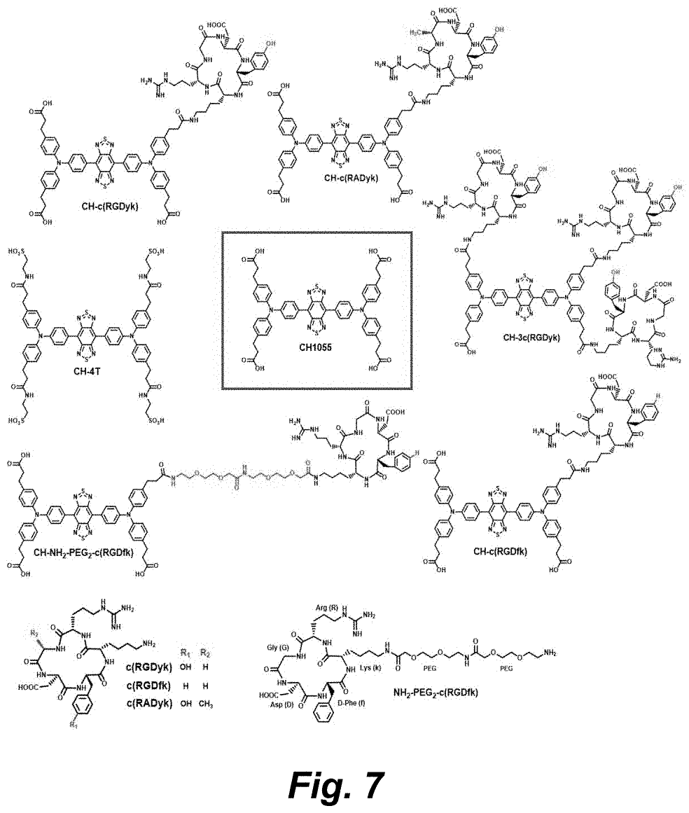

The syntheses of CH-c(RGDyk), CH-c(RGDfk), CH-c(RADyk), CH-PEG₂-c(RGDfk), CH-3c(RGDyk) were accomplished by EDC-mediated dehydration reaction of CH1055 dye and c(RGDyk), c(RGDfk), c(RAGyk), NH₂—PEG₂-c(RGDfk) (FIG. 2A, FIG. 7 for structures). From these candidates, CH-c(RDGyk) NPs and CH-c(RADyk) NPs (referred to as CH-RGD NPs and CH-RAD NPs, respectively) were chosen for further in vivo studies for its self-assembly characteristics and high quantum yield (QY) (FIGS. 2B-H, 8A-D).

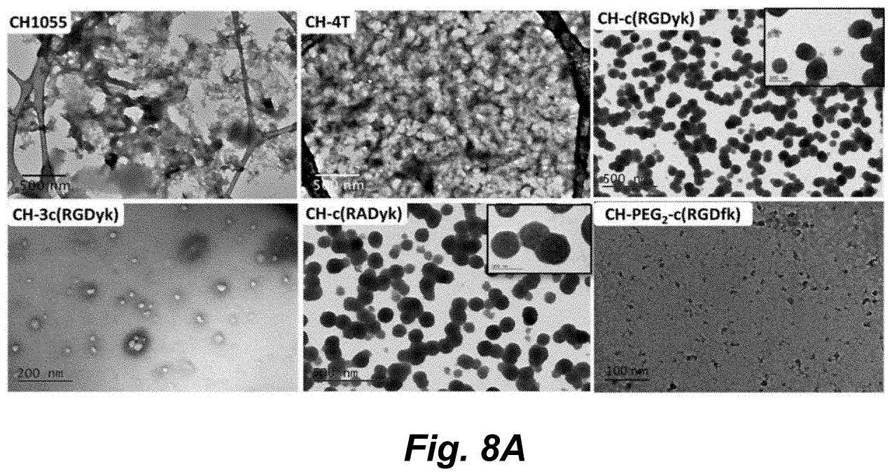

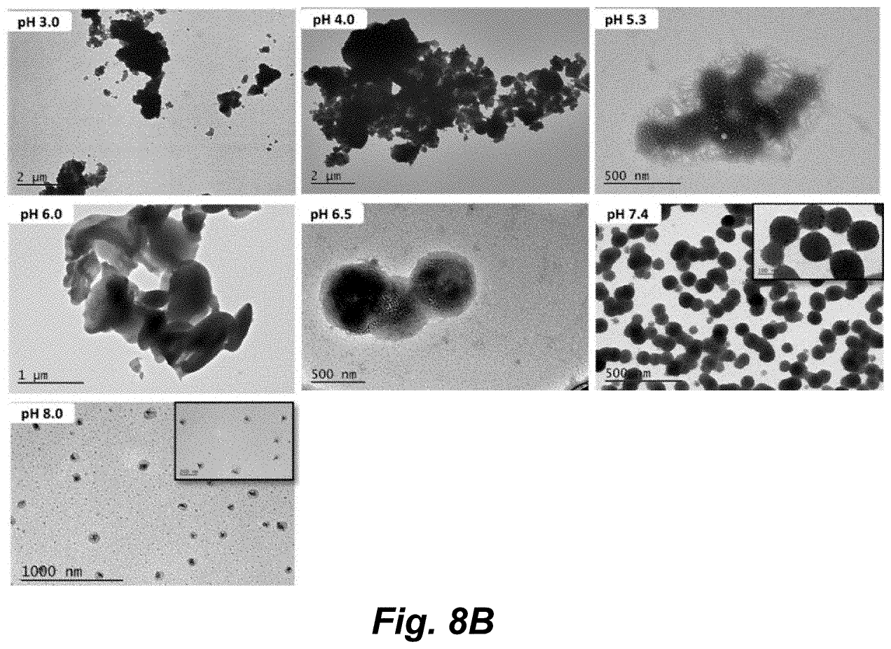

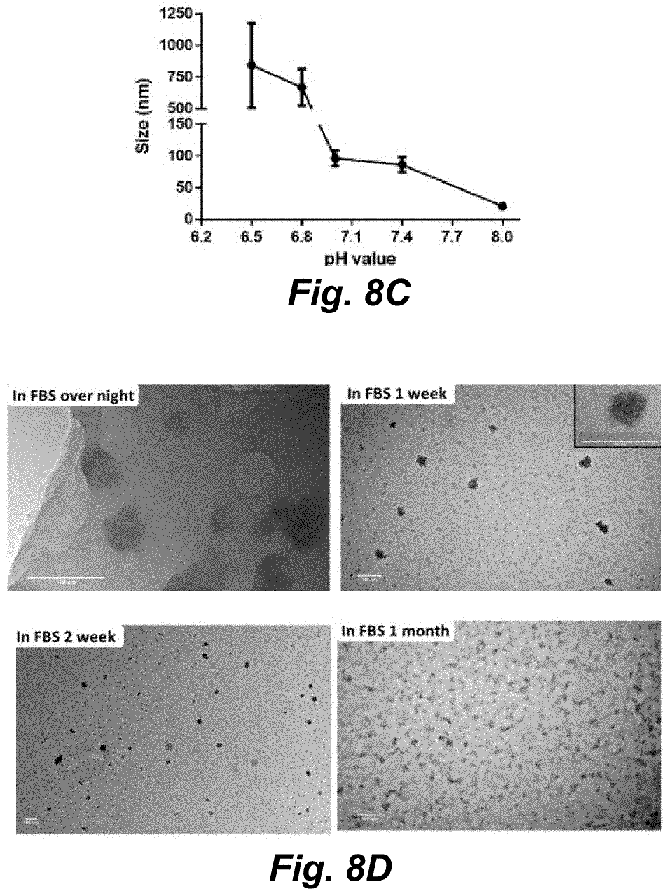

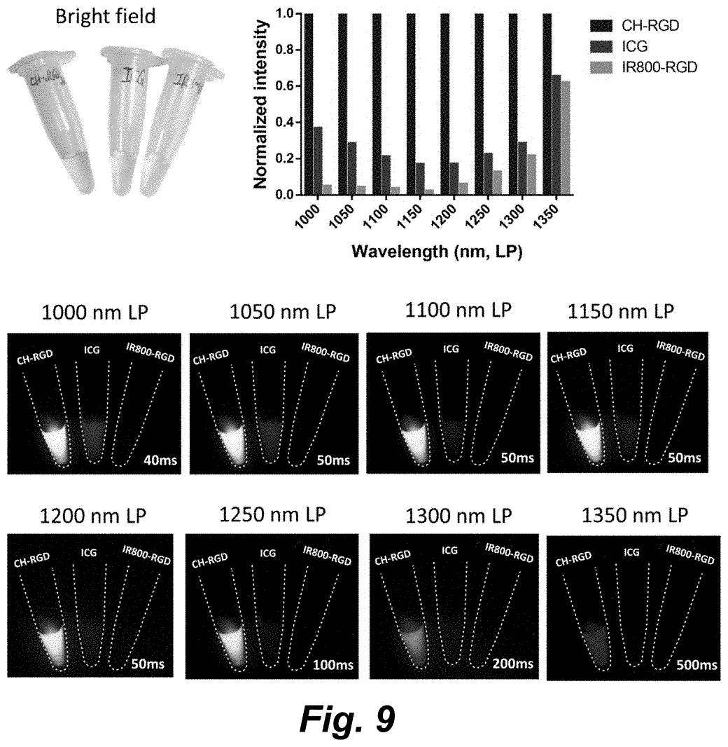

CH-RGD NPs self-assembled into approximately 80 nm solid nanospheres (FIGS. 1B, 8A-8D), as well as having a QY of 0.17% in water (FIG. 2H), several times higher than CH-4T, a previously studied NIR-II probe (Antaris et al. (2017) Nat. Commun. 8: 15269). Recently, ICG and IR800 dye-cores were found to have NIR-II fluorescent signals (Carr et al., (2018) Proc. Natl. Acad. Sci. U.S.A. 115: 4465-4470; Starosolski et al., (2017) PLoS One 12: e0187563). In parallel experiments, under all tested long-pass filters (1000-1350 nm), CH-RGD NPs were found to surpass ICG and IR800-RGD in fluorescence intensity (FIG. 9).

To investigate the pH dependent activity of CH-RGD NPs, 100 μM CH-RGD stock solution was added into pH 3.0, 4.0, 5.3, 6.0, 6.5, 7.4, 8.0 buffers. Self-assembled nanospheres were found at pH 6.5, 7.4, and 8.0 (FIG. 11, FIG. 8B). In slightly acidic solutions (lower than 7.0), CH-RGD further aggregated into larger (greater than 500 nm) NPs. Beyond pH 6.5, CH-RGD NPs aggregated into irregular precipitates, with fluorescence further increased by a factor of 2 (QY approximately 0.34%, FIGS. 1J, 1K, 8B, and 8C).

CH-RGD had a zeta potential of −36.4±1.7 at pH 7.4, dropping to −22.5±1.2 at pH 6.5. CH-RGD remains stable after 45 min of continuous laser excitation (0.33 W/cm², 808 nm) in water, pH 7.4 PBS, and FBS solutions (FIG. 2L). To investigate its long-term serum stability, 1 μM CH-RGD FBS solution was put into a 37° C. incubator for one month. Samples examined with DLS and TEM showed that the CH-RGD NPs were stable in FBS up to 2 weeks before degrading into smaller NPs. After one month, all CH-RGD NPs disassembled into less than 10 nm NPs (FIG. 2M and FIG. 8D).

In vitro bioactivity of CH-RGD was tested with macrophage, U87MG, and 143B cell lines, which all overexpress αᵥβ₃ integrin, to which RGD specifically binds (Antonov. et al., (2011) J. Cell Physiol. 226: 469-476; Huang et al., (2012) Clin. Cancer Res. 18: 5731-5740; Tome et al., (2013) Anticancer Res. 33: 3623-3627). After incubating these cells with 1 μM CH-RGD for 2.5 hours, there was evident cell membrane binding and receptor-mediated endocytosis (FIGS. 2F-2H). This specific binding could be blocked with 20× excess free RGD.

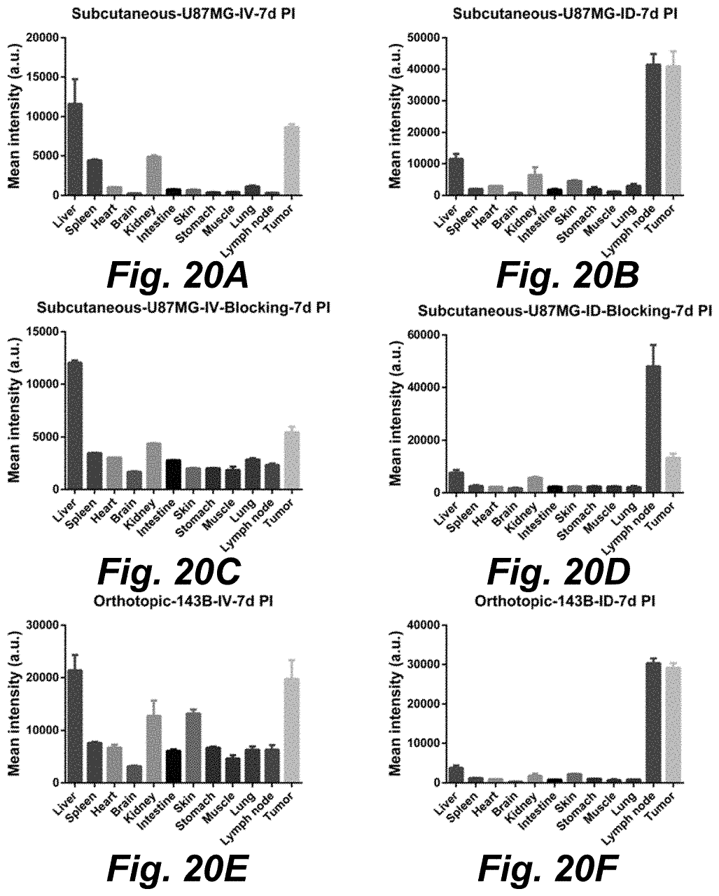

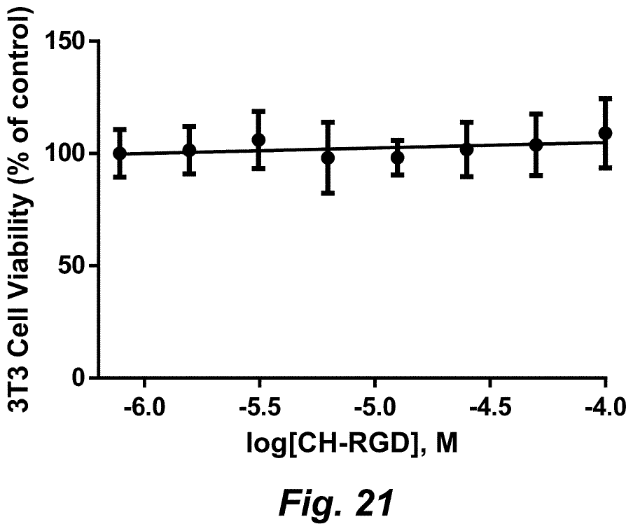

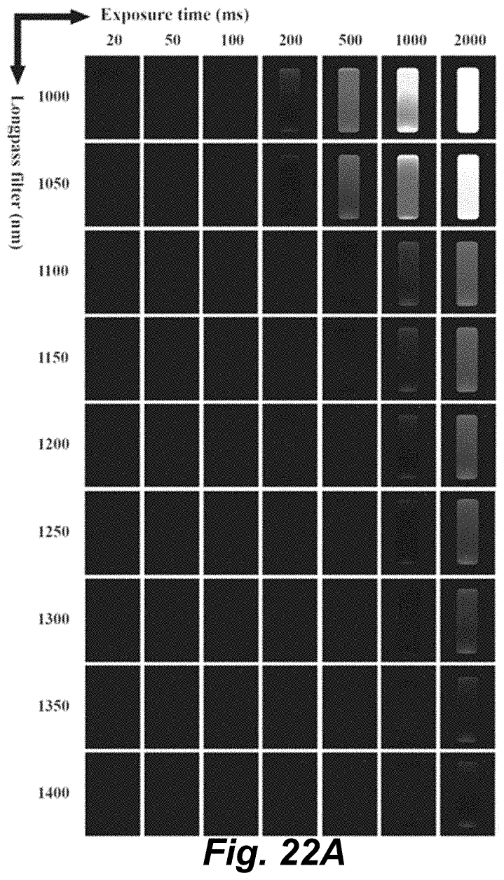

Adjusting the culture media pH from 7.4 to 6.5 resulted in large precipitates forming around the cells (FIG. 2P, white arrows). Similarly, CH-RGD aggregation can be observed in the endocytic organelles, which have a pH of 5.0-6.0 (Wang et al. (2014) Nat. Mater. 13: 204-212) (FIGS. 2N-2P, spots inside the cells). The toxicity of CH-RGD NPs was tested with 3T3 cells (FIG. 20A-20F). No obvious toxicity was found. Further in vivo studies confirmed its safety. Self-assembled CH-RGD NPs demonstrate αᵥβ₃ integrin active targeting and EPR passive targeting effects, which allow for better tumor accumulation. CH-RGD's well-defined chemical structure and simple assembly process significantly increase its clinical translation potential.

### Example 2

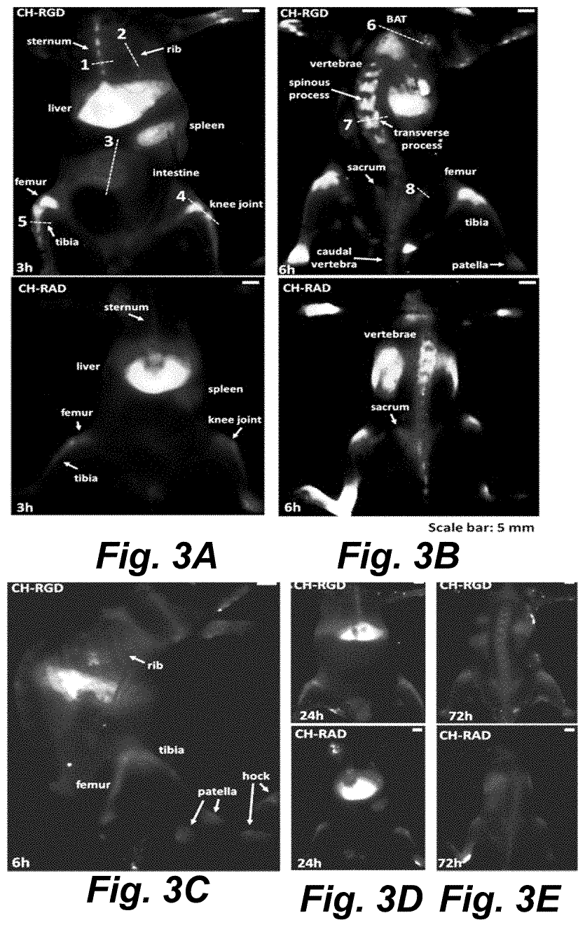

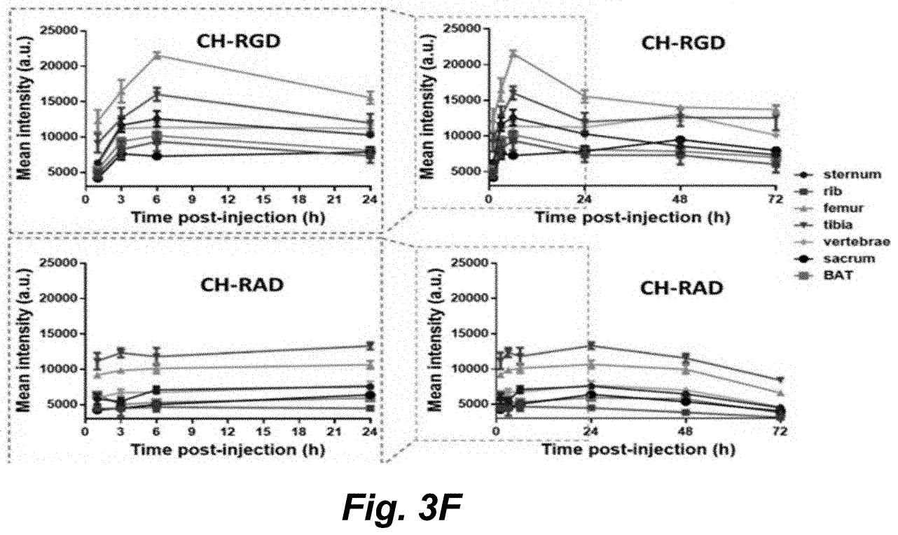

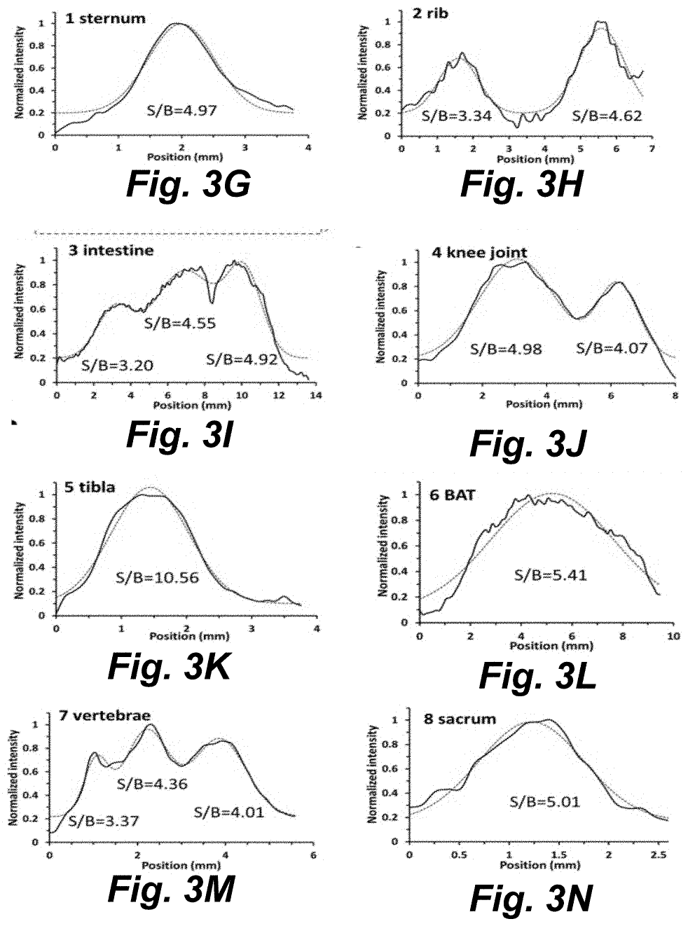

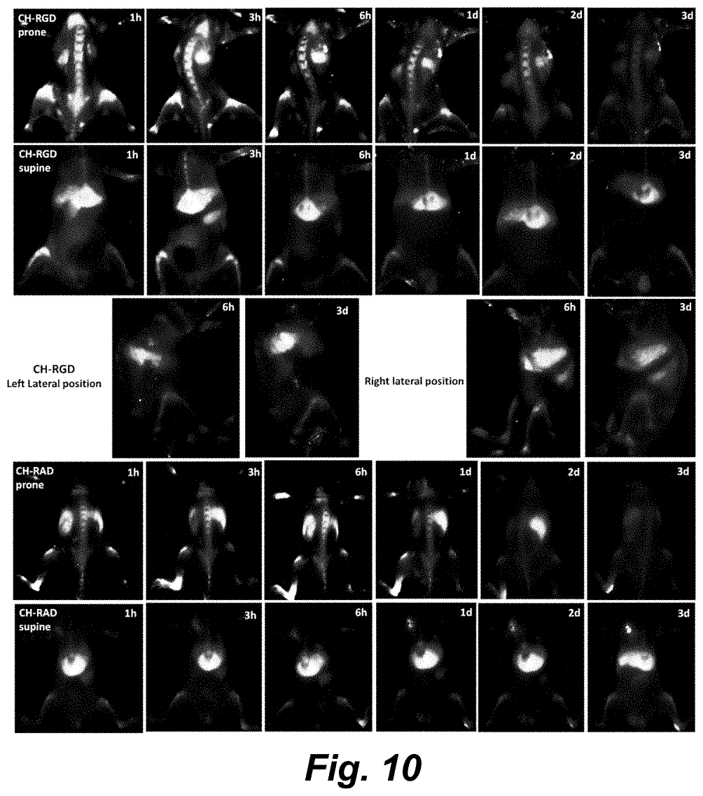

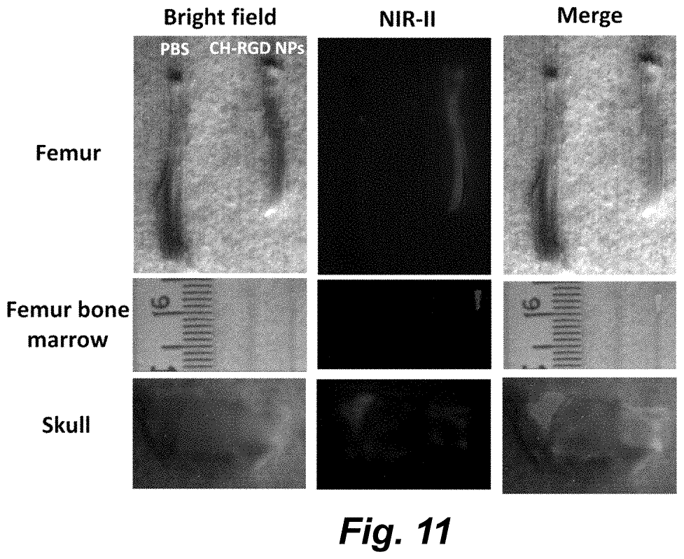

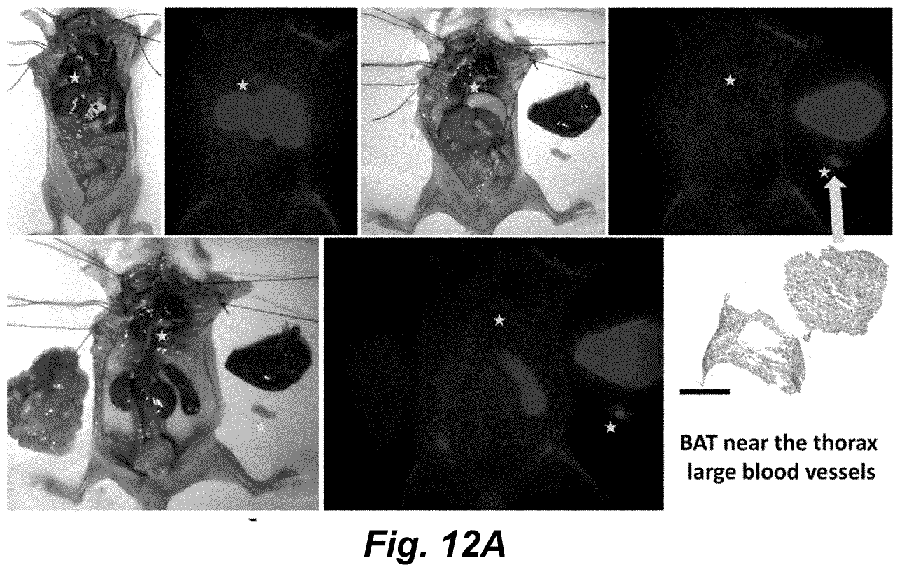

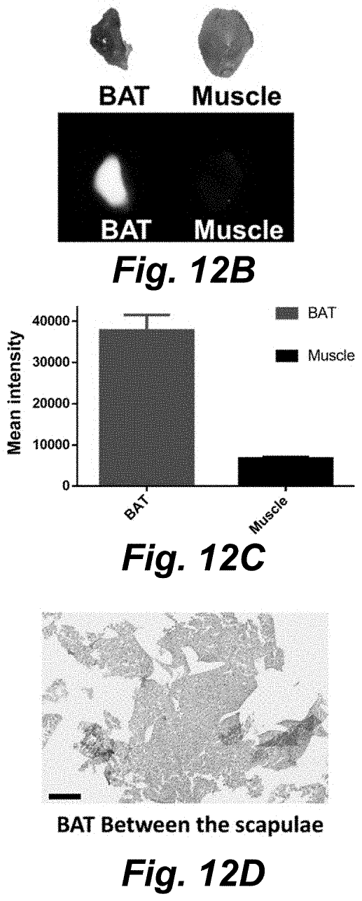

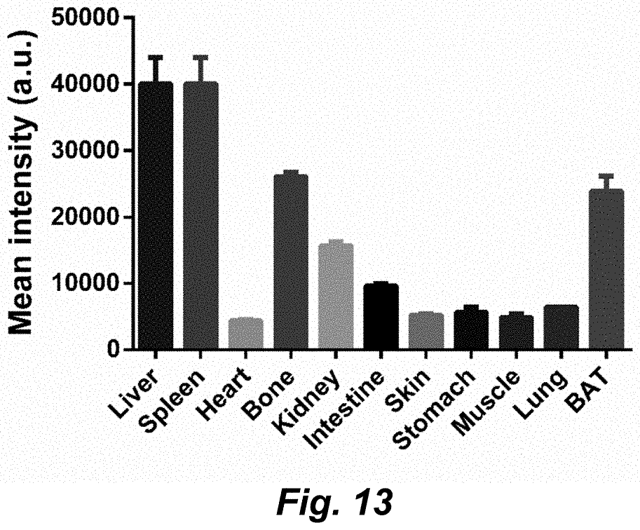

Self-assembly for bone imaging: Similar to macrophage, osteoclasts abundantly express αᵥβ₃ integrin (Nakamura et al., (1999) J. Cell Sci. 112: 3985-3993). CH-RGD NPs were successfully used for bone imaging. Bone NIR-II fluorescent signal was clearly observed 1 h post IV injection of 22 nmol CH-RGD NPs (FIG. 10). The bone signal reached peak intensity 6 h post-injection, (FIG. 3F). Most bones in vivo (FIGS. 3A-3C) and the skull (ex vivo, FIG. 11) can be clearly observed with a signal to background ratio (SBR) between about 3 to about 10 (FIGS. 3G-3N). The bone signal could still be visualized with in vivo imaging at 3 days post-injection, and ex-vivo imaging up to 45 days post-injection (FIG. 11). The intestine could also be visualized with high resolution (FIGS. 3A and 3I). Interestingly, brown adipose tissue (BAT) located between the scapulae (FIG. 3B) and large blood vessels in the thorax (ex-vivo, 3 days post-injection, FIGS. 12A-12D) also uptake CH-RGD NPs with a SBR approximately 5 and could be visualized 10 days post-injection (FIGS. 3B, 3I, and 12A-12D). CH-RGD NPs' eliminated half-life is approximately 7.65 d, calculated using the liver signal decay curve (FIG. 19).

To evaluate the influence of osteoclast αᵥβ₃ integrin targeting on bone imaging, CH-RAD NPs were IV injected into mice. Similar with CH-RGD NPs, signals were detected 1 h post-injection in bones (FIG. 10). Unlike CH-RGD NPs, however, CH-RAD NPs showed no dramatic accumulation in bone after 6 h post-injection (FIG. 3F, bottom curve).

The final concentration of CH-RAD NPs was about half that of CH-RGD NPs, measured by fluorescent intensity. CH-RAD NPs seemed to be heavily swallowed by phagocytic cells, accumulating in bone marrow after the first hour post-injection. Following this initial NP uptake, the fluorescence intensity was almost unchanged for the first 24H post-injection, then gradually excreted. It is possible, therefore, that αᵥβ₃ integrin targeting dramatically improved the probes' accumulation in bone. This provides an alternative to phosphonate (Fischer et al., (1996) Am. J. Physiol. 270: H358-363) or oligopeptide (Hyun et al., (2014) Angew Chem. Int. Edit. 53: 10668-10672; Zhang et al., (2012) Nat. Med. 18: 307-314) related bone imaging methods.

### Example 3

Self-Assembly for Blood, Lymphatic Vessel and Lymph Node Imaging:

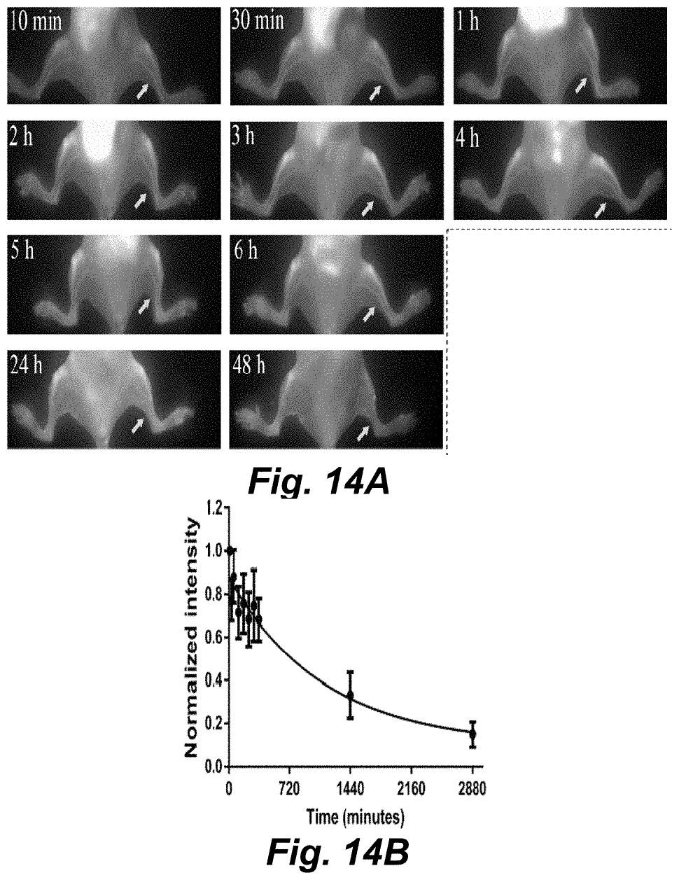

Intraoperative fluorescence vascular imaging is useful in rapid assessment of vascular anastomosis during organ transplantation surgery (Sekijima et al. (2004) Transplant Proc. 36: 2188-2190). A FDA approved NIR-1 dye, ICG is commonly used clinically to accomplish this task (Arichi et al., (2014) Transplant Proc. 46: 342-345), which is limited by a short blood half-life (3-5 mins) (De Gasperi et al., (2016) World J. Hepatol. 8: 355-367) and superficial imaging depth (Antaris et al., (2016) Nat. Mater. 15, 235-242). It was found that NIR-II fluorescent CH-RGD NPs show improved vascular imaging quality and longer half-life (approximately 776 mins, FIG. 14A-14B) compared to ICG. The clinically-used ICG dose is about 645 nmol/kg (0.5 mg/kg) (De Gasperi et al., (2016) World J. Hepatol. 8: 355-367). As a parallel study, the experimental dosage was approximately 640 nmol/kg.

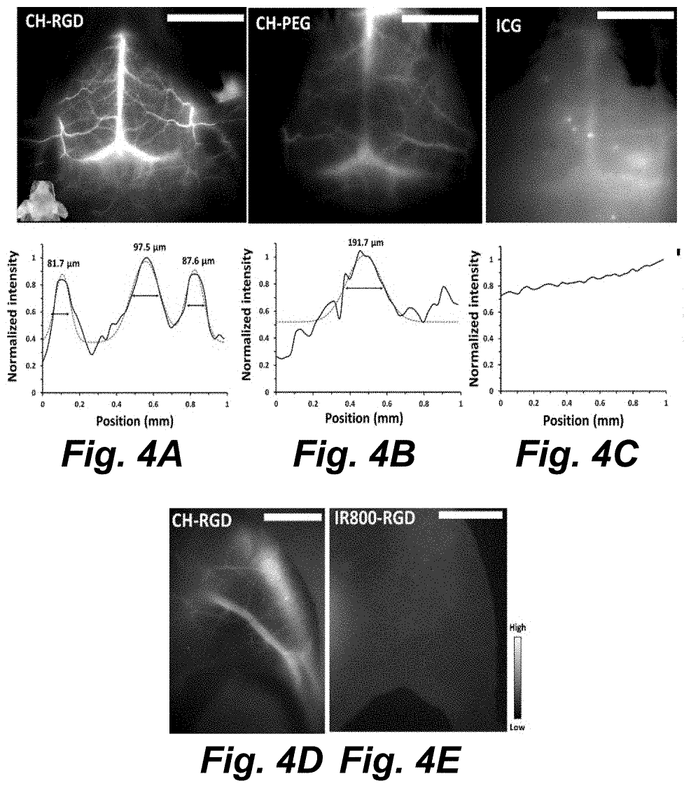

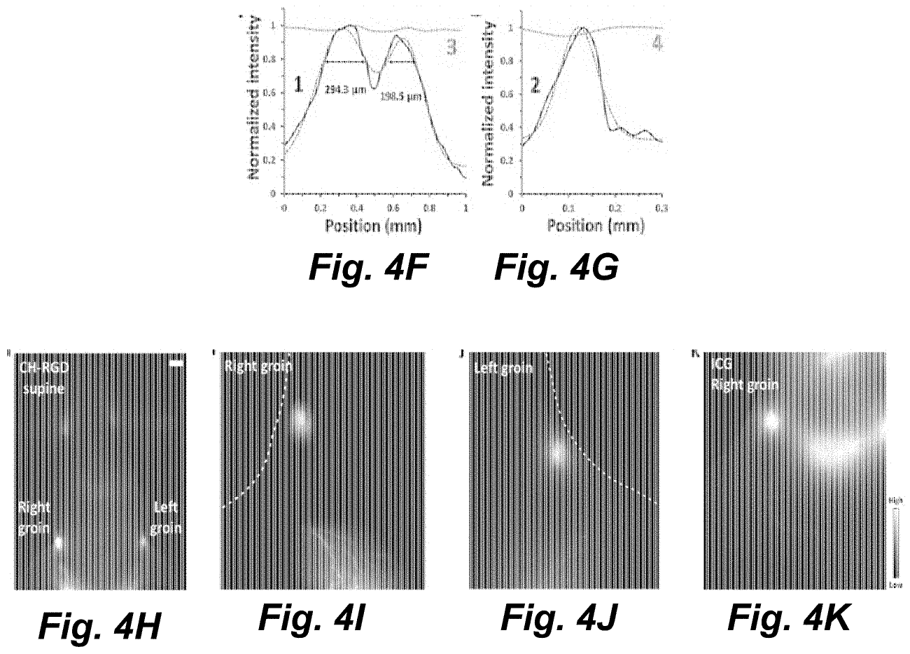

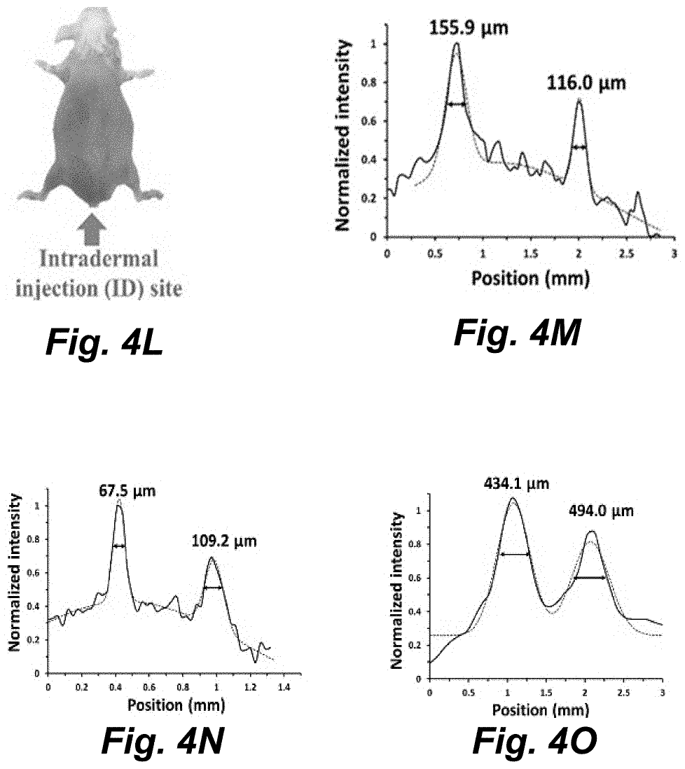

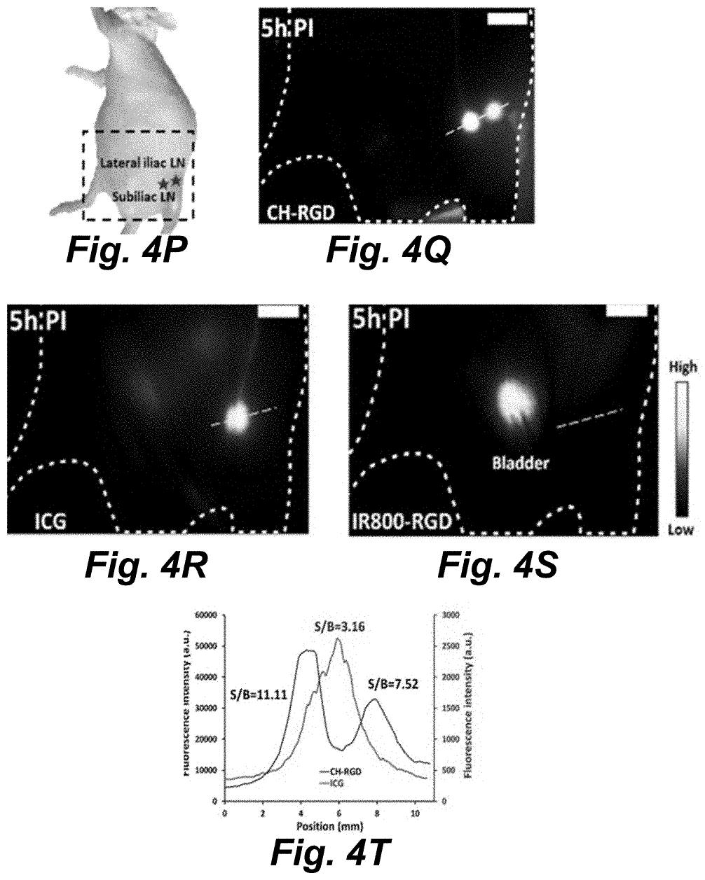

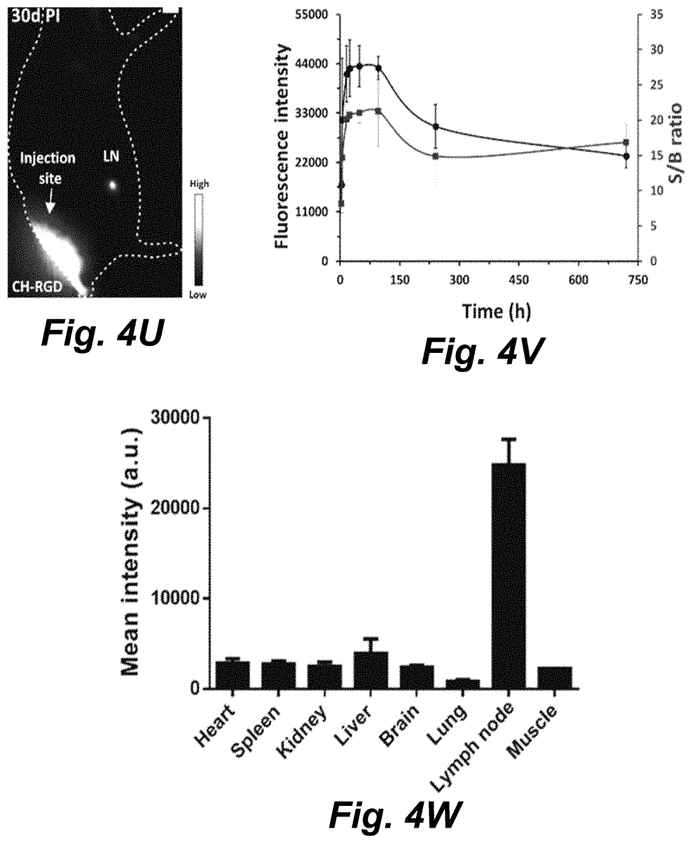

CH-RGD resolution was high enough to identify brain blood vessels (less than 100 μm, 2-4 mm depth), leg capillaries (less than 200 μm), and to distinguish between the close-proximity femoral artery and vein (FIGS. 4A, 4D, 4F, and 4G). With ICG, only the largest brain blood vessels could be detected in NIR-1 (FIG. 4C). With IR800-RGD, no blood vessels could be detected (FIGS. 4E-4G). Moreover, CH-RGD improves on formerly published CH-PEG (Antaris et al., (2016) Nat. Mater. 15, 235-242), which could only detect approximately 200 μm brain blood vessels (FIG. 4B).

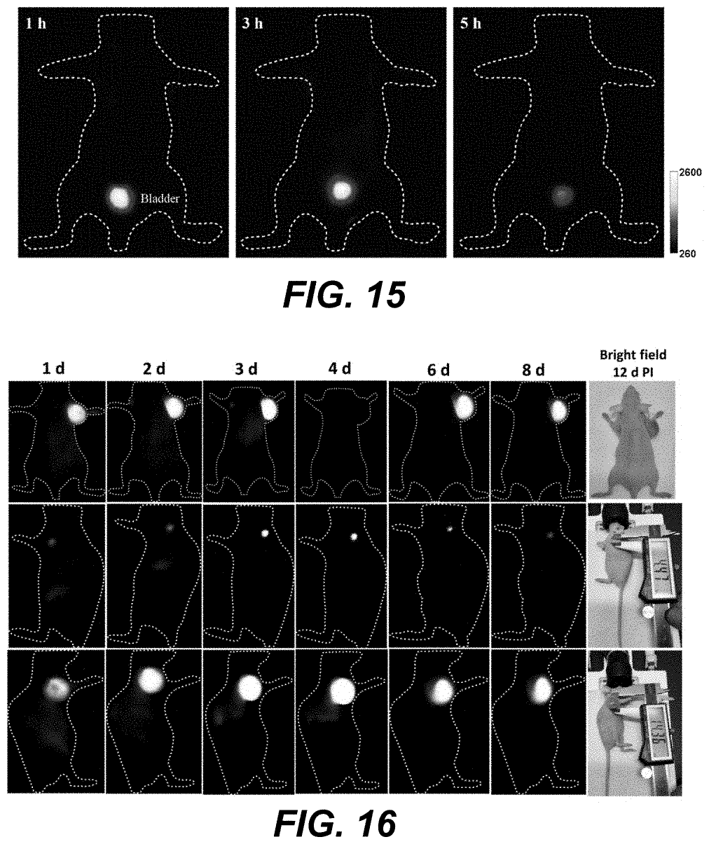

The lymphatic system, despite playing an important role in disease, lacks non-invasive monitoring methods (Adamczyk et al., (2016) Virchows Arch. 469: 3-17). Here, CH-RGD NPs demonstrate effective lymphatic vessel and lymph node imaging, due to its high macrophage uptake and NIR-II fluorescent properties. 67.5 μm lymphatic capillaries (Fischer et al., (1996) Am. J. Physiol. 270: H358-363) in mice groins were detected (FIGS. 4H-4J, 4M, and 4N) after ID injection (FIG. 4L). In comparison, only greater than 400 μm lymphatic vessels could be detected by ICG (FIGS. 4K and 4O), and none were detected by IR800-RGD (FIG. 15).

CH-RGD probes can isolate lymph nodes in older, overweight mice, despite interference by fatty tissue of the fluorescence signal (Mathieu & Labrecque (2012) J. Vis. Exp. e3444). One-year old, male, overweight BALB/c mice were selected for testing. Fluorescent imaging was measured 5 h after ID injection of equal dosages CH-RGD NPs, ICG and IR800-RGD (FIGS. 4I, 4P). CH-RGD NPs and ICG accumulated in mice lymph nodes. However, the deep lateral iliac lymph node (approximately 4 mm under skin, FIGS. 4P, 4Q) can only been seen with CH-RGD NP injected mice, with a full width at half maximum (FWHM) of 2.6 mm and a SBR of approximately 7.5 (FIG. 4Q, 4R, 4T) (Shao et al., (2013) J. Immunol. Methods 389: 69-78). For subiliac lymph node imaging, the FWHM was 1.8 mm with a SBR of approximately 11 and a steeper slope (leftmost peak, FIG. 4T).

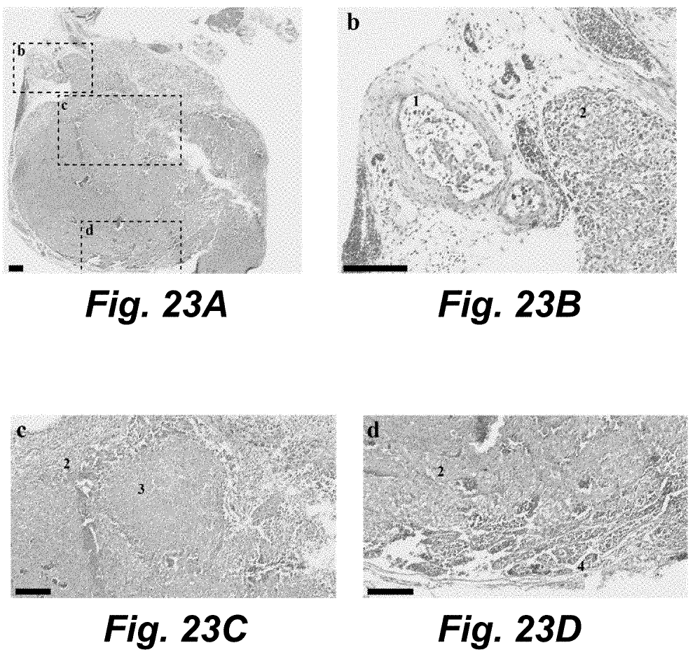

In contrast, ICG gave a 2.8 mm FWHM, SBR of approximately 3 with a broader slope (red curve, FIG. 4T). IR800-RGD, after 1 h post ID injection, accumulated in the bladder, leading to no signal in the lymph node or lymphatic vessels (FIG. 4S). CH-RGD NPs can also be used for long term lymph node labeling. The subiliac lymph node was imaged 30 d post-injection by in vivo NIR-II imaging with a SBR of 16.8±2.6 (FIG. 4V). CH-RGD NPs exhibited the greatest lymph node fluorescence intensity at 4 days post-injection with a SBR of 21.3+5.0, which is far beyond the Rose criterion of SBR=5 for distinguishing image features (Antaris et al., (2016) Nat. Mater. 15, 235-242). This allowed NIR-II fluorescent imaging guided lymph node resection with a short exposure time (100 ms, FIGS. 23A-23D). The mice organs were harvested 30 d post-injection to acquire the biodistribution of CH-RGD NPs, which showed most accumulated in lymph nodes, followed by the liver (FIG. 4W).

### Example 3

Aggregation for Subcutaneous Tumor Imaging:

CH-RGD N Ps demonstrated specific, durable accumulation due to αᵥβ₃ integrin targeting, pH-responsive enhanced aggregation, and the EPR effect. While nanomedicine is thought to benefit from the EPR effect, its impact remains variable (Matsumura & Maeda (1986) Cancer Res. 46: 6387-6392; Nakamura et al., (2016) Bioconjugate Chem. 27: 2225-2238; Kufe, D. W. Cancer Medicine 6 review: Companion to Holland-Frei Cancer Medicine-6. (Decker, 2003)).

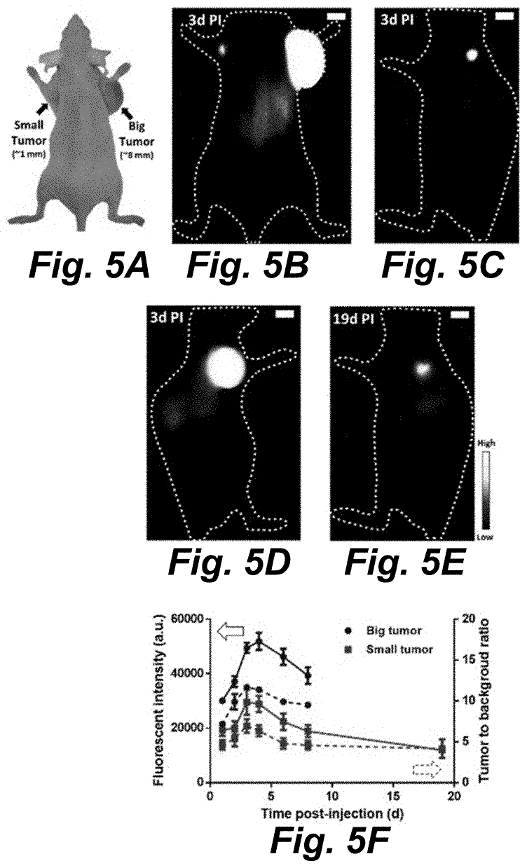

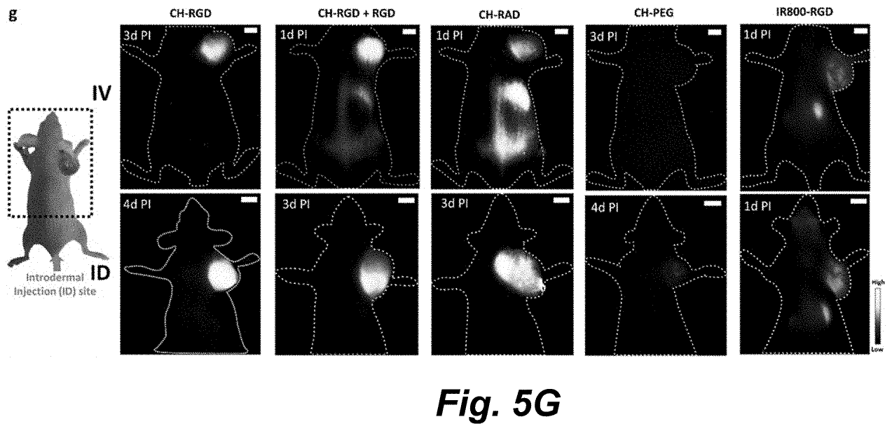

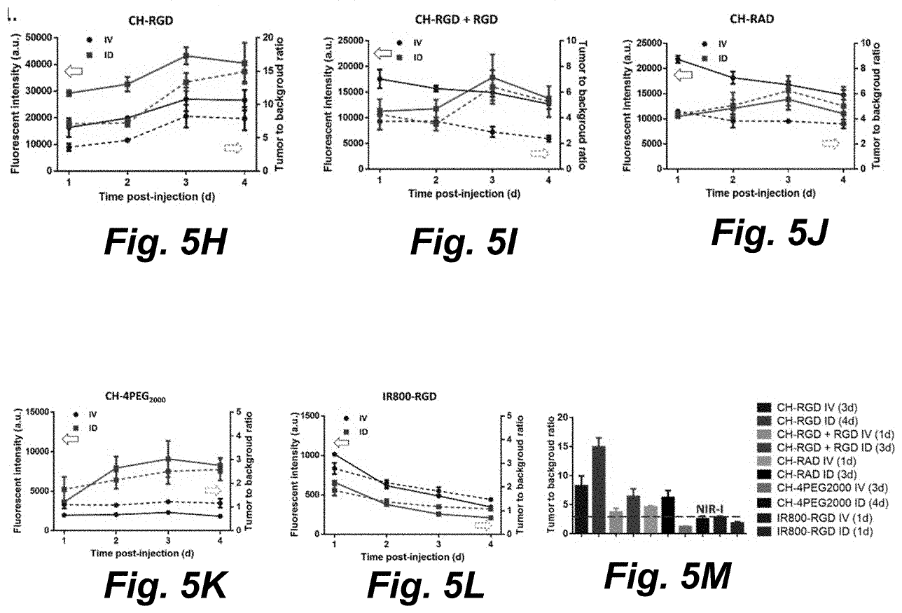

To distinguish the influences of integrin targeting, EPR, and pH-responsive aggregation on tumor accumulation, double tumor models were made (FIG. 5A). 10 nmol CH-RGD NPs were IV-injected into mice when the two tumors reached diameters of approximately 1 mm (tumor volume approximately 0.52 mm³) and approximately 6 mm (tumor volume approximately 113 mm³). Faster tumor accumulation of the CH-RGD NPs was found at the approximately 6 mm tumor 3 days post-injection (FIG. 5F). Tumor fluorescence was detectable approximately 0.5 h post-injection for the larger tumor and 4 h post-injection for the smaller tumor. Average fluorescence intensity decreased between 3 days and 4 days post-injection for the small tumor (FIGS. 5B, 5C, 5F, and 16), but peaked at 4 days post-injection in the large tumor (FIG. 5F), implying extravasation of CH-RGD exceeding uptake in the former. With increasing tumor size, decreasing mean pH is detected (Tannock & Rotin (1989) Cancer Res 49: 4373-4384). Extravasation seems restricted by the increased, pH-responsive aggregation of CH-RGD in the larger tumor. The low pH, increased tumor volume and limited fluorescence enhancement suggest a limited EPR effect, in line with research dissuading the overreliance of nanomedicine on EPR (Nakamura et al., (2016) Bioconjugate Chem. 27: 2225-2238).

The small tumor can still be observed 19 days post injection with a tumor to background ratio (TBR) of approximately 4 (FIG. 5E), a decrease from the highest TBR (approximately 6) at 3 days post-injection. The larger tumor had a TBR of approximately 10 at 4 days post-injection. The pH-dependent aggregation seemed to prolong the probe retention time in the tumor, which can be beneficial for cancer therapy drugs. Overall, due to RGD-mediated integrin targeting, the EPR effect, and pH-sensitive aggregation, CH-RGD demonstrates a higher TBR (approximately 10) than current NIR-I alternatives (approximately 3) (Huang et al., (2012) Clin. Cancer Res. 18: 5731-5740).

Further testing of the EPR effect was done to compare CH-RGD to other fluorescent probes, as well as investigating injection methods (IV vs ID) on u87MG tumors. CH-RAD and CH-RGD NPs with 20× excess free RGD (to block the RGD targeting) were used to determine the probe's reliance on the EPR effect. Through IV administration, both CH-RGD/RGD and CH-RAD reached the tumor 1 day post-injection exclusively through the EPR effect. The maximum tumor fluorescence and TBR occurred at 1 day post-injection (FIGS. 5I and 5J). Comparison of the mean tumor fluorescent intensity of CH-RGD (40 k a.u.) with CH-RGD/RGD (17K a.u.) and CH-RAD (22 k a.u.) (FIGS. 5H-5J) shows that the EPR effect is responsible for about half of tumor accumulation.

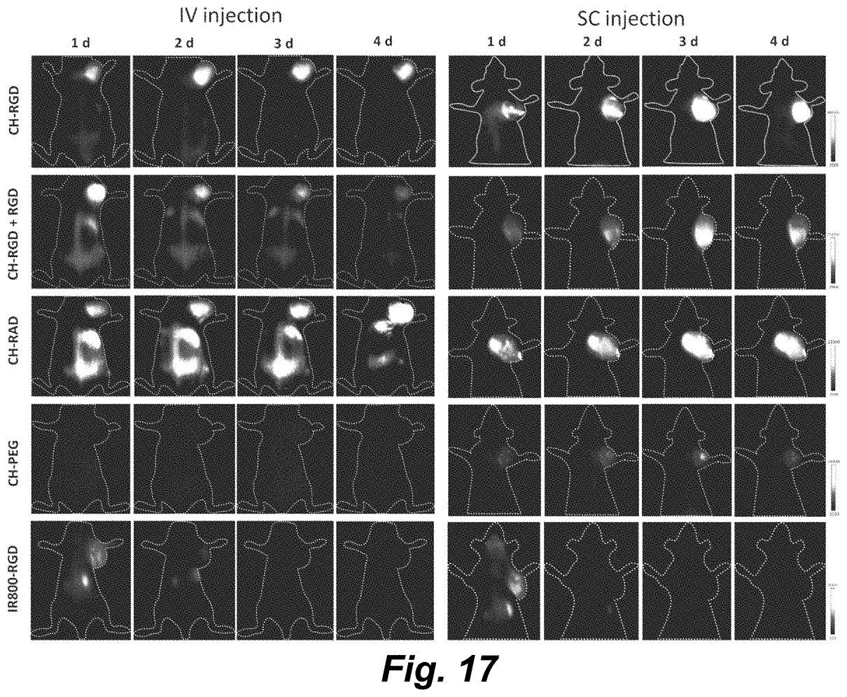

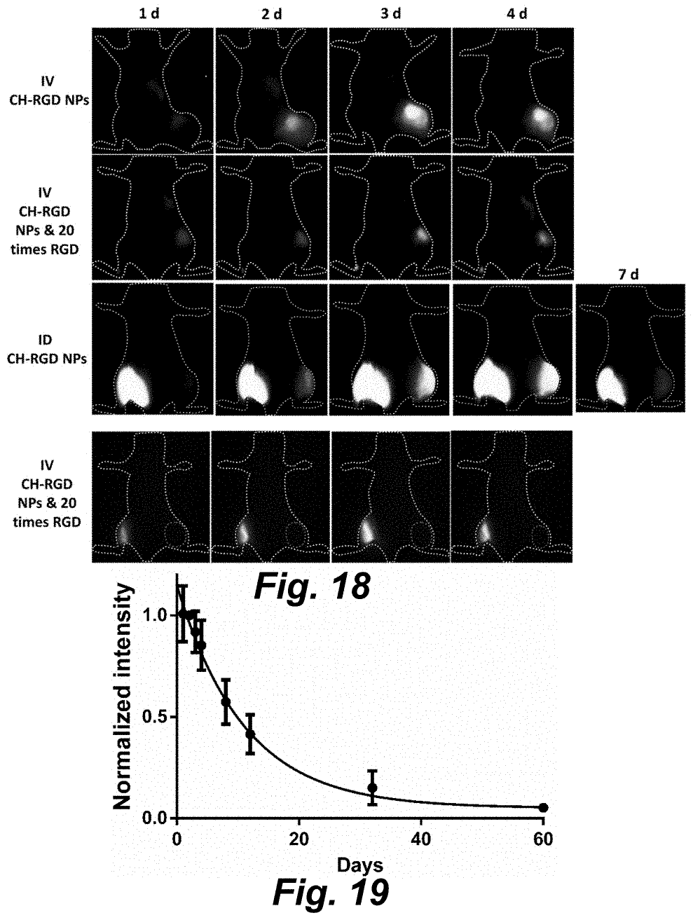

From a dynamic point of view, after 1 day post-injection, all probes achieved similar mean tumor intensities and TBR (FIGS. 5H-5J), but only CH-RGD NPs increased further in the next 3 days. This demonstrates that active binding of RGD to αᵥβ₃ integrin may prolong tumor accumulation time, resulting in better tumor fluorescence. CH-PEG and IR800-RGD were used as parallel control probes. CH-PEG targeted the tumor only through the EPR effect, while IR800-RGD used the αᵥβ₃ integrin targeting effect. A 5 nmol dose was less than half the dosage as used previously for CH-PEG (Antaris et al., (2016) Nat. Mater. 15, 235-242), which results in only a slight tumor signal (FIGS. 5G and 17). IR800-RGD was clearly observed 1 day post-injection with a peak TBR of approximately 2.7, agreeing with previous studies (Huang et al., (2012) Clin Cancer Res 18: 5731-5740). The results show that CH-RGD NPs are the best overall tumor targeting agent with a TBR of approximately 7 after IV injection (FIGS. 5G-5M, and 17).

IR800-RGD showed similar results whether IV or ID injected (FIG. 5L). However, CH-RGD NPs showed higher accumulation in subcutaneous brain tumors (TBR approximately 15, 4 days post-injection, FIG. 5H) with ID injection compared to IV injection. Testing with 20× excess of free RGD and CH-RAD showed that αᵥβ₃ integrin binding contributed to accumulation (FIG. 5G, CH-RGD+RGD, 51). All CH1055 derivatives, CH-RGD, CH-RAD and CH-PEG-showed better TBR through ID injection. This may be caused by the high macrophage uptake of CH1055 probes, evidenced by the cell imaging results (FIG. 2N).

ID injection presents the probe to immune cells including macrophages, prompting uptake to attempt to remove the foreign material (Chong et al., (2013) Front. Immunol. 4). Given the longer absorption time of ID injection, the same therapeutic effect can be had at a lesser dose (Kenney et al., (2004) New Engl. J. Med. 351: 2295-2301). ID-injected CH-RGD NPs demonstrated the highest yet TBR of approximately 15, ultra-bright tumor fluorescent signals (FIGS. 5G and 5H) and low organ signals (FIG. 20A-20F). The low liver signal implies few free CH-RGD NPs reach the blood without being engulfed. Ultra-bright lymphatic system signals indicated that most CH-RGD NPs were engulfed by immune cells, which eventually circulate in the blood, accumulating at the tumor site due to an immune response (Adam et al., (2003) Pharmacol. Therapeut. 99: 113-132) (FIG. 1B). Since accumulation of CH-RGD NPs relies in part on an immune response, they can potentially be used as a low cost way of monitoring cancer immunotherapies (Sharma, P. (2010) Proc. Natl. Acad. Sci. U.S.A. 107: 13977-13978).

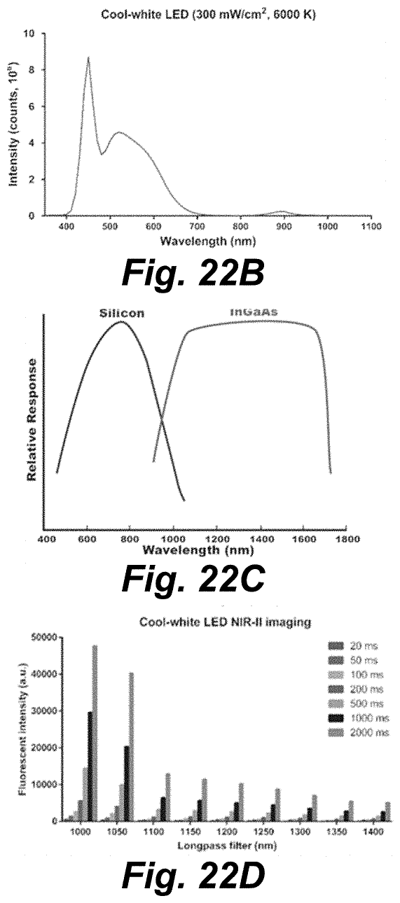

Traditional NIR-I fluorescent imaging (Si detector) guided surgery need low visible light environments to enhance the S/B. To keep brighter surgery environments, complicated hardware and software are added to the imaging guided surgery setup (DSouza et al., (2016) J. Biomed. Opt. 21: 80901). While conventional LED light (Pimputkar et al., (2009) Nat. Photonics 3: 179-181) (less than 800 nm) has a negligible effect on NIR-II imaging (InGaAs detector) (FIG. 22A-22D), allowing NIR-II image-guided surgery to occur under bright light with simpler setup than that of NIR-I. Due to CH-RGD NPs high TBR and tumor fluorescence, the first ID injection mediated NIR-II fluorescent imaging guided tumor resection was performed with short exposure time (100 ms).

### Example 4

Aggregation for Orthotopic and Metastatic Tumor Imaging:

CH-RGD was further tested with orthotopic tumor models. Orthotopic glioblastomas (U87MG) were used to test IV injection while orthotopic and metastatic bone cancer (143B) models were applied to test the ID injection route.

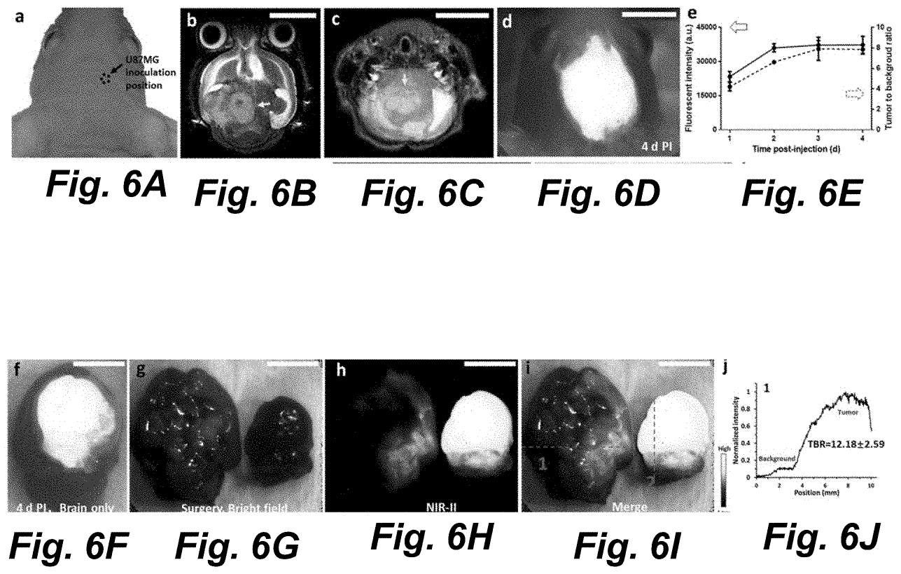

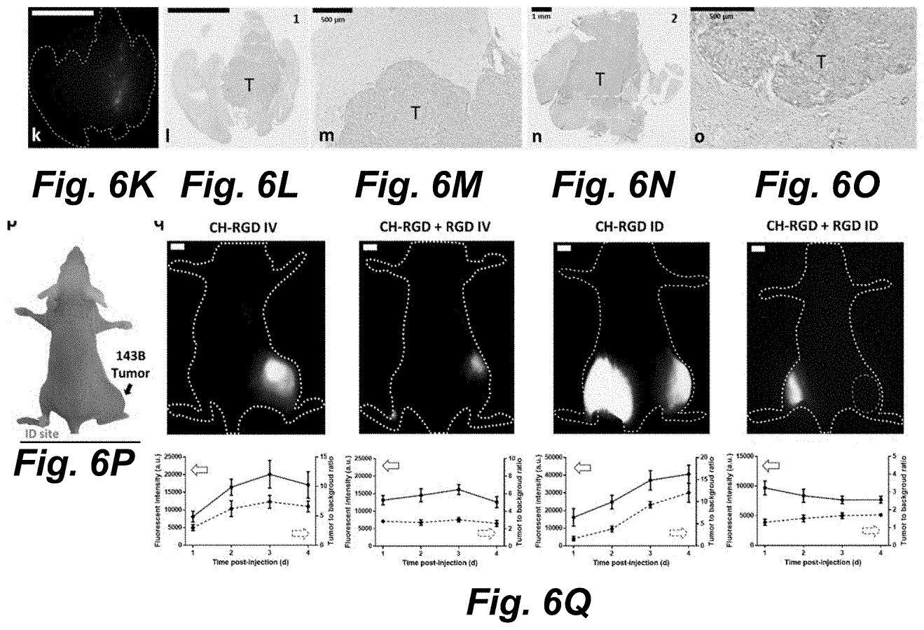

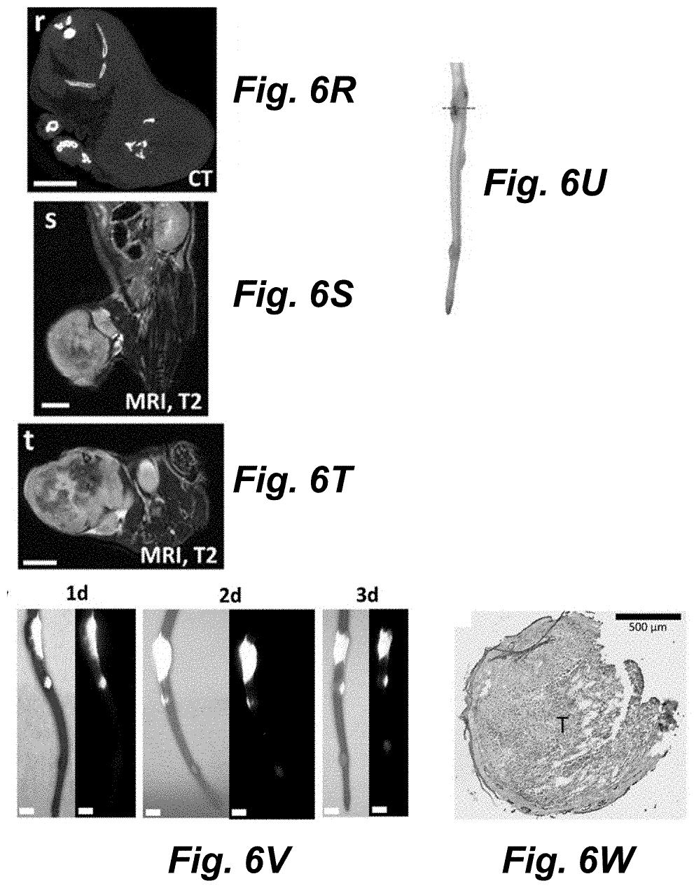

The peak glioblastoma fluorescence signal was measured 3 days post IV injection with a TBR of approximately 8 (FIGS. 6D, 6E), surpassing the previously reported approximately 3 TBR with IR800-RGD (Huang et al., (2012) Clin. Cancer Res. 18: 5731-5740). MRI imaging confirmed the glioblastoma shape and position (FIGS. 6B and 6C). Ex-vivo glioblastoma imaging was done to confirm the accumulation of CH-RGD NPs. The ex-vivo brain imaging clearly shows the shape of the tumor (FIGS. 6F-6I) with a TBR of approximately 12 (FIG. 6J). Aggregation of CH-RGD NPs can be observed in histological sections as micron-sized red spots (FIG. 6K). Pathological analysis (FIG. 6L-6O) supported the NIR-II fluorescent imaging results (FIG. 6I), showing the fluorescent tumors as distinguishable from the background (FIG. 6J). Based on its high accumulation in glioblastomas, CH-RGD NPs show potential for image-guided brain tumor surgery.

Cancer metastases to bone are common and severe, prompting investigation into applying CH-RGD NPs, based on their high subcutaneous tumor accumulation when ID injected (Ratanatharathorn et al., (1999) Int. J. Radiat. Oncol. 44: 1-18). 143B cells were inoculated in the right tibia of mice. Micro-CT and MRI were used to identify the orthotopic bone tumors (FIGS. 6R-6T). CH-RGD was ID injected, with CH-RGD IV injection and 20× excess free RGD tests applied as parallel controls. ID injection showed the greatest tumor accumulation with the highest fluorescence intensity and TBR (approximately 12) at 4 days post-injection (FIG. 6Q, CH-RGD ID) and was still identifiable 7 days post-injection. The high fluorescence intensity enabled a 100 ms exposure time, allowing for NIR-II imaging guided bone cancer surgery.

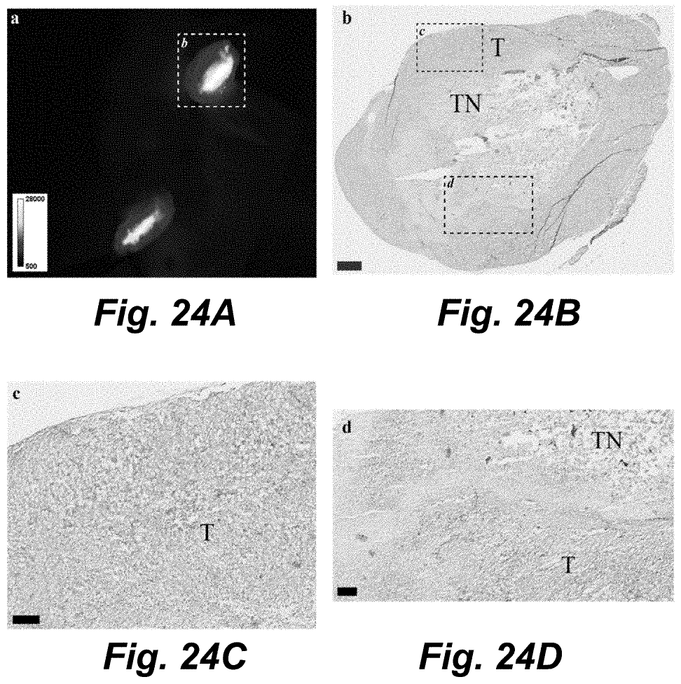

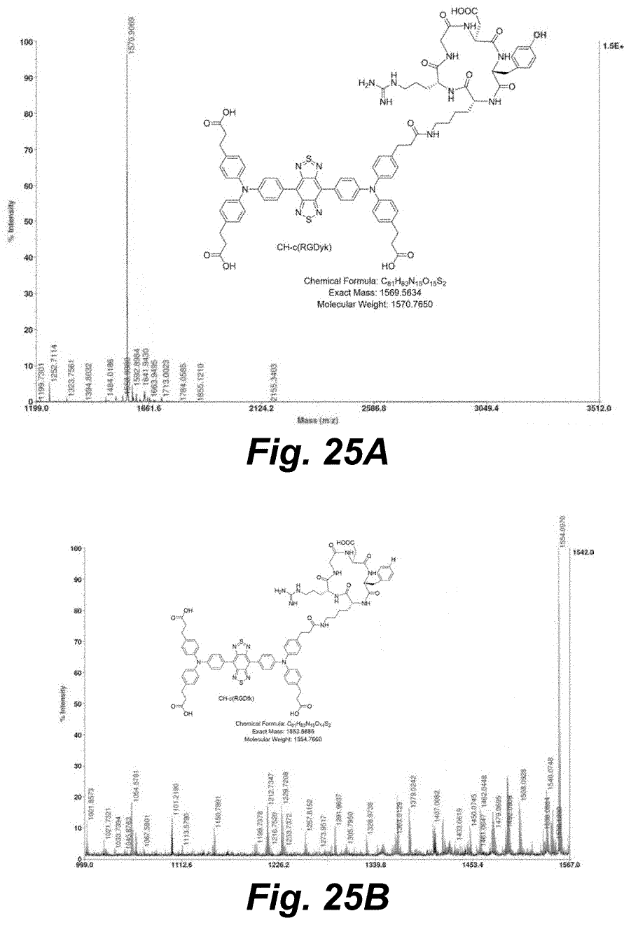

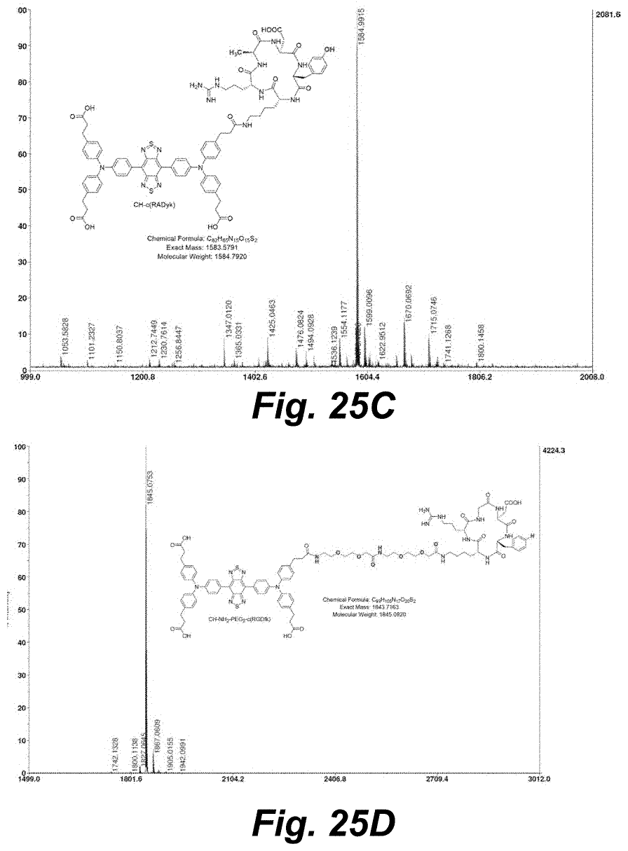

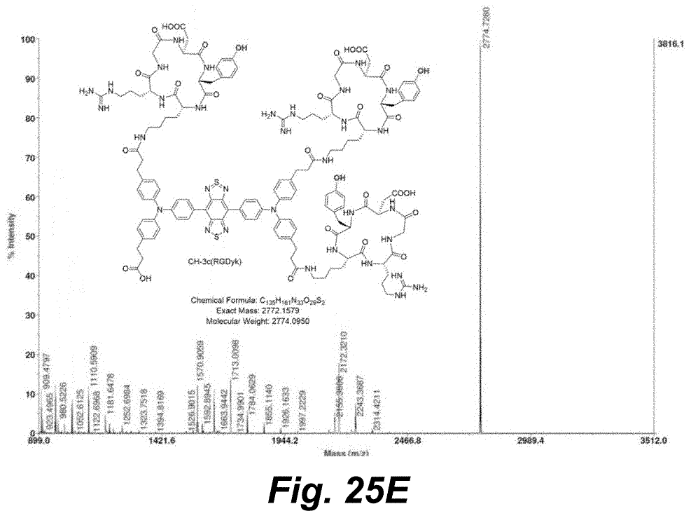

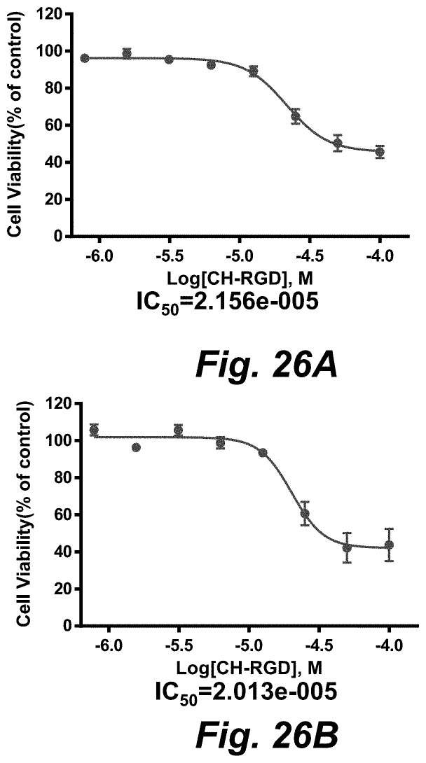

Strong NIR-II fluorescence was found at the tumor necrosis area, showing pH-responsive aggregation of CH-RGD NPs (FIG. 24A-24D). IV injection results (FIG. 6Q) were similar to that of the subcutaneous tumor model (FIG. 5G). Orthotopic bone tumors showed dim imaging quality due to the tumor depth. After incision, the bone tumors (FIG. 6Q, CH-RGD+RGD IV or ID) had similar fluorescence to the subcutaneous brain tumors (FIG. 5G, CH-RGD+RGD IV or ID). The versatility of ID injected CH-RGD NPs was further tested in metastatic bone cancer models. CH-RGD NPs were ID injected into the mouse left hind limb when the metastatic tail bone tumor grew to approximately 1 mm. The tumors were identified 1 day post-injection with similar intensity and TBR as the subcutaneous U87MG tumors (FIG. 5G, CH-RGD, ID), supporting the generalizability of CH-RGD NPs for metastatic bone tumor mapping.

### Example 5

CH-c(RGDyk), CH-c(RGDfk), CH-c(RADyk), CH-PEG₂-c(RGDfk) Synthesis:

CH1055 (1 equiv.), NHS (2.0 equiv.), EDC (1.2 equiv.) dissolve in dry DMSO, room temperature reacting 1.5 h. Then add RGD (c(RGDyk), c(RGDfk), c(RADyk), NH₂—PEG₂-c(RGDfk)) (1.5 equiv.), DIPEA (20 equiv.), room temperature reacting overnight. Dissolve the reaction mixture in water, quench for 0.5 h. Then use HPLC for purification: C18 column, acetonitrile and water as mobile phase. Gradient: 5% acetonitrile to 95% acetonitrile in 35 mins. MALDI-TOF-MS was used to identify the product. MALDI-TOF-MA calculated for: CH-c(RGDyk) [C₈₁H₈₃N₁₅O₁₅S₂] (M.W.): 1,570.8. Found: 1,570.9. Yield 81%; CH-c(RGDfk) [C₈₁H₈₃N₁₅O₁₄S₂] (M.W.): 1,554.7. Found: 1,554.1. Yield 73%; CH-c(RADyk) [C₈₂H₈₅N₁₅O₁₅S₂] (M.W.): 1,584.8. Found: 1,584.9. Yield 77%; CH-PEG₂-c(RGDfk) [C₉₃H₁₀₅N₁₇O₂₀S₂] (M.W.): 1,845.1. Found: 1,845.1. Yield 80%.

### Example 6

CH-3c(RGDyk) Synthesis:

CH1055 (1 equiv.), NHS (4.0 equiv.), EDC (3.3 equiv.) dissolve in dry DMSO, room temperature reacting 2 h. Then add c(RGDyk) (3.5 equiv.), DIPEA (60 equiv.), room temperature reacting overnight. Dissolve the reaction mixture in water, quench for 0.5 h. Then use HPLC for purification: C18 column, acetonitrile and water as mobile phase. Gradient: 5% acetonitrile to 95% acetonitrile in 35 mins. MALDI-TOF-MS was used to identify the product. MALDI-TOF-MA calculated for: CH-3c(RGDyk) [C₁₃₅H₁₆₁N₃₃O₂₉S₂] (M.W.): 2,774.1. Found: 2,774.7. Yield 22.3%.

### Example 7

Self-Assembled Cyclopeptide-CH1055 NPs Preparation:

CH-c(RGDyk), CH-c(RGDfk), CH-c(RADyk), CH-PEG₂-c(RGDfk), CH-3c(RGDyk), CH-4PEG₂₀₀₀ and CH-4T were dissolved in distilled water at the concentration of 637 μM (1 mg/mL) as stock solutions. Then the stock solutions were dilute into 500, 250, 125, 62.5, 31.25, 15.625, 7.8125, 1 μM solutions and placed in dark overnight at room temperature for sufficient self-assembly. Then all solutions include stock solutions were diluted into appropriate concentrations for DLS and TEM analysis study. All other analysis: absorption, fluorescent spectrum measurement etc. used the corresponding diluted stock solutions.

100 μM CH-c(RGDyk) (CH-RGD) was diluted into 10, 5, 1 μM solution by different pH buffers for pH responds self-assembly study. The DLS, TEM and fluorescence measurements were also performed in the relevant pH buffer.

CH-RGD was dissolved in 1×PBS pH 7.4 buffer with the concentration of 637 μM as the stock solution for in vivo imaging study. Specified volume of this stock solution was diluted into 200 μL (IV injection) or 50 μL (IP injection) solution (diluted by 1×PBS pH 7.4 buffer) for mice in vivo studies.

### Example 8

In Vitro Serum Stability:

Fetal Bovine Serum (FBS, Gibco® Catalog #26140) was used to dilute the CH-RGD PBS stock solution into 2 mL, 10 μM mixture solution. And the mixture was incubated at 37° C. in a cell culture incubator for one month. At each designated time point, 200 μL serum mixture were collected for DLS and TEM analysis.

### Example 9

Animal Handling:

Teen-week old BALB/c mice (n=3 male and 3 female per group) were obtained from Charles River for bone imaging studies, teen-week old C57BL/6 female mice (n=4 per group) were obtained from Charles River for brain vessel studies, teen-week old NU/NU female mice (n=4 per group) were obtained from Charles River for leg vessel studies, teen-week old BALB/c male mice (n=4 per group) were obtained from Charles River and housed for another 42 weeks for lymph node and lymphatic vessel studies, eight-week old female NU/NU mice (n=3 per group) were obtained from Charles River for tumor targeting studies. Before vessel or tumor imaging, all mice were anaesthetized in a rodent anesthesia machine with 2 l min⁻¹ O₂ gas mixed with 3% Isoflurane. Tail vein injection of contrast agents were carried with a catheter and synchronized with a camera that started continuous image acquisition simultaneously. The IV injected dose was a 200 μL bolus in a 1×PBS solution at specified concentrations. The ID injected dose was a 50 μL bolus in a 1×PBS solution at specified concentrations. During the time course of imaging the mouse was kept anaesthetized by a nose cone delivering 2 l min⁻¹ 02 gas mixed with 3% isoflurane. The sample sizes of mice were selected based on previously reported studies. Mice were randomly selected from cages for all experiments. No blinding was performed.

### Example 10

Optical Characterization:

The NIR fluorescence spectrum was taken using a home-built NIR spectroscopy setup. The excitation source was a 0.25 W 808 nm laser. The excitation laser was filtered with a combination of an 850/1,000/1,100/1,200/1,300 nm short-pass filters (Thorlabs). The excitation light was allowed to pass through the solution sample in a 1-cm-path-length cuvette (Starna Cells) and the resulting emission filtered through a 900 nm long-pass filter (Thorlabs) to reject the incident excitation laser light. The emitted light was directed into a spectrometer (Princeton Instruments, IsoPlane 160) equipped with a two-dimensional InGaAs array (Princeton Instruments, NIRvana TE 640). Spectra were corrected post-collection to account for the sensitivity of the detector and extinction feature of the filter. Photostability was determined by spiking CH-RGD NPs into FBS, 1× pH 7.4 PBS and DI water, and exposing it in a 1-cm-path-length cuvette (Starna Cells) to continuous 808 nm excitation at a power density of 0.33 W cm⁻² and taking images every 5 mins for approximately 45 min. Stability was determined by measuring the ROI and comparing the fluorescence intensity against the starting fluorescence signal.

Fluorescence enhancement is defined as

(∫₉₀₀¹⁵⁰⁰I_{CH-RGD}d_{λ})/(∫₉₀₀¹⁵⁰⁰Iₓd_{λ}),

where Iₓ corresponds to the fluorescent emission spectrum of other CH1055 derivatives. All fluorescent enhancement values were derived from measurements on the wavelength-corrected photospectrometer unless specifically stated otherwise.

### Example 11

### Xenograft Tumor Implantation

### Single Tumor Model:

Nude mice were purchased from Charles River. U87MG cells were cultured in Dulbecco's modified Eagle medium containing high glucose (Gibco), which were supplemented with 10% fetal bovine serum and 1% penicillin-streptomycin. The cell was expanded in tissue culture dish and kept in a humidified atmosphere of 5% CO₂ at 37° C. The medium was changed every other day. A confluent monolayer was detached with 0.5% trypsin, and dissociated into a single-cell suspension for inoculation. When the mice reached eight weeks in age, mice were inoculated with 1×10⁶ U87MG cells on the right front shoulder. The tumor was allowed to grow for approximately 30 days before imaging, at which point they were approximately 30-120 mm³ in volume.

Double Tumors Model:

The first right shoulder big tumor implantation is the same as above description. The difference is allowed the tumor grow for 2 to 3 weeks. When the right tumor diameter (ø) was between about 1 to 2 mm, 1×10⁶ U87MG cells were injected into the mice left shoulder. After about 1 to 2 weeks, when the left shoulder tumor had grown to a diameter of about 1 mm and the right shoulder tumor was approximately 6 mm in diameter, the mouse was used for imaging.

### Example 12

U87MG Orthotopic Brain Tumor Implantation:

Eight-week old female nude mice (Charles River) were anaesthetized using 2.0% isoflurane and positioned in a Benchmark (Leica) stereotactic instrument. The top of the mouse head was cleaned with 70% ethanol and betadine. Ophthalmic ointment was applied. A sagittal skin incision of 0.5 cm was made over the bregma and the skull was exposed. A burr hole in the right hemisphere was drilled according to the coordinate 0.5 mm anterior and 2.0 mm lateral to the bregma. A 10 μl gas-tight syringe (Hamilton) with a 26-gauge needle (Cat 800010) was inserted to the striatum and lowered to a depth of 2.5 mm from the dural surface. U87 MG-Luc cells (5 μl, 2×10⁴ cells in PBS) were injected into the striatum over 15 min using a microsyringe pump controller (World Precision Instruments). The same amount of PBS was also injected as an experimental control. The needle was left for 10 min before being withdrawn. The burr hole was sealed with bone wax (Cat CPB31A) to prevent leakage of cerebrospinal fluid, and the scalp was closed with a tissue adhesive (Surgibond®). Animals were used for experiments after ten days, when tumors had reached a size of approximately 2 mm diameter as determined by MRI.

### Example 13

143B Orthotopic Osteosarcoma Tumor Implantation:

Eight-week old female nude mice (Charles River) were anaesthetized using 2.0% isoflurane and positioned on a heating pad (42° C.). The shoulder was cleaned with 70% ethanol and betadine. 1×10⁶ 143B cells were first injected subcutaneously into the shoulder mice and the tumor allowed to grow to a diameter 5-8 mm solid tumor. The tumors were surgical resected and cut into 1×1×1 mm³ pieces.

The right tibia of 8-week old NU/NU mice was opened in the central part by drilling with a dental drill for a 0.5 mm diameter hole. One tumor fragment was insert into the hole and contact with the bone marrow. Then the surgical wound was sutured. Mice were observed daily for tumor growth. Palpation, micro CT and 3T MRI were used to monitor the tumor growth. The mouse was used for imaging experiment, when the tumor size had reached to 3-5 mm diameter.

### Example 14

Bone Metastasis Animal Models:

When the orthotopic 143B tumor grows to 3-4 mm diameter, surgery was performed to resect the tumor. Tumor was cut into pieces, and the visible tumor fragments were taken out. Then the surgical wound was sutured. Although the metastasis was not frequently occurring in mice tail, mice tail metastasis is the easiest diagnostic metastasis. By visual inspection, 143B tail metastasis mouse was chosen out. The mouse was used for imaging experiment when the tumor size reach to approximately 1 mm diameter.

### Example 15

NIR-II Imaging:

Mice were placed on a stage with a venous catheter for IV injection of contrast and imaging agents. 0.5 mL syringe was used directly for ID injection. All NIR-II images were collected on a 640×512 pixel two-dimensional InGaAs array (Princeton Instruments, NIRvana TE 640). The excitation laser was an 808 nm laser diode at a power density of approximately 140 mW cm⁻². Emission was typically collected with 1000, 1050, 1100, 1150, 1200, 1250, 1300, 1350, 1400 nm LP filter (Thorlabs). 50 mm and 75 mm prime lens (Edmund optics) was used to obtain magnifications ranging of 1× (whole body) to 2.5× (high magnification) magnification by changing the relative position of camera, lens and animals. A binning of 1 and a variable exposure time were used for the InGaAs camera (640×512 pixel) to capture images in the NIR-II window. Images were processed with ImageJ.

### Example 16

NIR-I Imaging:

All NIR-I images were collected A 1,344×1,024-pixel Hamamatsu silicon CCD detector (Hamamatsu C8484-03G02; pixel number: 1,344×1,024; readout noise: approximately 7 electron r.m.s.; dark current: 0.01 electrons per second per pixel) with an 814-851 nm bond pass filter (Edmund, #84-107). The excitation laser was a 785 nm laser diode at a power density of approximately 140 mW cm⁻² with an 800 nm short pass filter (Thorlabs). 50 mm and 75 mm prime lens (Edmund optics) was used to obtain magnifications ranging of 1× (whole body) and 2.5× (high magnification) magnification by changing the relative position of camera, lens and animals. A binning of 1 and a variable exposure time were used for the silicon CCD camera (1,344×1,024 pixel) to capture images in the NIR-I window. Images were processed with ImageJ.

### Example 17

Brain Tumor NIR-II Imaging:

Brain tumor imaging was performed using the high-magnification NIR-II set-ups. NIR-II imaging was performed with a variety of filters and exposure times. For brain tumor imaging, a 1200 LP filter was employed with an exposure time ranging from 200 to 2000 ms. Mice were imaged 1, 2, 4, 8, 24, 48, 72, and 96 hours post-injection.

### Example 18

Cytotoxicity of CH-RGD NPs:

The CH-RGD NPs toxicity was determined in vitro with a MTT assay (3-(4,5-dimethylthiazol-2-yl)-2,5-diphenyl tetrazolium bromide, Sigma-Aldrich, St. Louis, Mo.) on NIH/3T3 cells. Approximately 5,000 cells were incubated per well with 200 μL of DMEM growth medium and serially diluted CH-RGD NPs solution (n=6 for each concentration). The cells were kept at 37° C. in a humidified atmosphere containing 5% CO₂ for 48 h in the presence of CH-RGD NPs at different concentrations. Immediately before addition of 20 μl of MTT (5 mg/mL), a colorimetric indicator of cell viability, the CH-RGD NPs-spiked medium was removed from each well plate and replaced with 180 μL fresh medium. After 4 h, the medium was carefully removed, 100 μL DMSO was added. Shaking for 10 mins, the color change was quantified using a microplate reader (TECAN Infinite M100) and taking absorbance readings at 570 nm with a reference wavelength of 650 nm. Cell viability was plotted as a fraction of the absorbance of control wells incubated without CH-RGD NPs.

### Example 19

Synthesis of CH-c(RGDyk):

Dissolve 1 mg CH1055 (1.03 μmol) in 100 μl of dry DMSO. Then 0.24 mg NHS (2.06 μmol), 0.24 mg EDC (1.236 μmol) were added and the mixture reacted for 1.5 h at room temperature. Then 0.96 mg c(RGDyk) (1.545 μmol), 3.6 μl DIPEA (2.66 mg, 20.6 μmol) were added. The reaction solution was stirred overnight at room temperature under a nitrogen atmosphere. After the reaction finished, 100 μl of water was added and stirred for 1 h to quench the reaction. Finally, Dionex Summit high-performance liquid chromatography (HPLC) system (Dionex Corporation, Sunnyvale, Calif., USA), 340U four-channel ultraviolet-visible absorbance detector, Dionex C4, 9.4 mm×250 mm semi-preparative column, gradient elution starting from 5% acetonitrile and ending up with 95% acetonitrile (in water) at 35 min, 3 ml min⁻¹ flow rate, 254 nm and 650 nm detection wavelength was used to purify the reaction. Overall, 1.31 mg CH-c(RGDyk) (yield 81%) was produced as a green solid. MALDI-TOF-MS was used to identify the product. MALDI-TOF-MS calculated for: CH-c(RGDyk) [C₈₁H₈₃N₁₅O₁₅S₂] (M.W.): 1,570.8. Found: 1,570.9.

### Example 20

Synthesis of CH-c(RGDfk):

Dissolve 1 mg CH1055 (1.03 μmol) in 100 μl of dry DMSO. Then 0.24 mg NHS (2.06 μmol), 0.24 mg EDC (1.236 μmol) were added and the mixture reacted for 1.5 h at room temperature. Then 0.93 mg c(RGDfk) (1.545 μmol), 3.6 μl DIPEA (2.66 mg, 20.6 μmol) were added. The reaction solution was stirred overnight at room temperature under a nitrogen atmosphere. After the reaction finished, 100 μl of water was added and stirred for 1 h to quench the reaction. Finally, Dionex Summit high-performance liquid chromatography (HPLC) system (Dionex Corporation, Sunnyvale, Calif., USA), 340U four-channel ultraviolet-visible absorbance detector, Dionex C4, 9.4 mm×250 mm semi-preparative column, gradient elution starting from 5% acetonitrile and ending up with 95% acetonitrile (in water) at 35 min, 3 ml min⁻¹ flow rate, 254 nm and 650 nm detection wavelength was used to purify the reaction. Overall, 1.17 mg CH-c(RGDfk) (yield 73%) was produced as a green solid. MALDI-TOF-MS was used to identify the product. MALDI-TOF-MS calculated for: CH-c(RGDfk) [C₈₁H₈₃N₁₅O₁₄S₂](M.W.): 1,554.7. Found: 1,554.1.

### Example 21

Synthesis of CH-c(RADyk):

Dissolve 1 mg CH1055 (1.03 μmol) in 100 μl of dry DMSO. Then 0.24 mg NHS (2.06 μmol), 0.24 mg EDC (1.236 μmol) were added and the mixture reacted for 1.5 h at room temperature. Then 0.98 mg c(RADyk) (1.545 μmol), 3.6 μl DIPEA (2.66 mg, 20.6 μmol) were added. The reaction solution was stirred overnight at room temperature under a nitrogen atmosphere. After the reaction finished, 100 μl of water was added and stirred for 1 h to quench the reaction. Finally, Dionex Summit high-performance liquid chromatography (HPLC) system (Dionex Corporation, Sunnyvale, Calif., USA), 340U four-channel ultraviolet-visible absorbance detector, Dionex C4, 9.4 mm×250 mm semi-preparative column, gradient elution starting from 5% acetonitrile and ending up with 95% acetonitrile (in water) at 35 min, 3 ml min⁻¹ flow rate, 254 nm and 650 nm detection wavelength was used to purify the reaction. Overall, 1.26 mg CH-c(RADyk) (yield 77%) was produced as a green solid. MALDI-TOF-MS was used to identify the product. MALDI-TOF-MS calculated for: CH-c(RADyk) [C₈₂H₈₅N₁₅O₁₅S₂] (M.W.): 1,584.8. Found: 1,584.9.

### Example 22

Synthesis of CH-PEG₂-c(RGDfk):

Dissolve 1 mg CH1055 (1.03 μmol) in 100 μl of dry DMSO. Then 0.24 mg NHS (2.06 μmol), 0.24 mg EDC (1.236 μmol) were added and the mixture reacted for 1.5 h at room temperature. Then 1.38 mg NH₂—PEG₂-c(RGDfk) (1.545 μmol), 3.6 μl DIPEA (2.66 mg, 20.6 μmol) were added. The reaction solution was stirred overnight at room temperature under a nitrogen atmosphere. After the reaction finished, 100 μl of water was added and stirred for 1 h to quench the reaction. Finally, Dionex Summit high-performance liquid chromatography (HPLC) system (Dionex Corporation, Sunnyvale, Calif., USA), 340U four-channel ultraviolet-visible absorbance detector, Dionex C4, 9.4 mm×250 mm semi-preparative column, gradient elution starting from 5% acetonitrile and ending up with 95% acetonitrile (in water) at 35 min, 3 ml min⁻¹ flow rate, 254 nm and 650 nm detection wavelength was used to purify the reaction. Overall, 1.52 mg CH-PEG₂-c(RGDfk) (yield 80%) was produced as a green solid. MALDI-TOF-MS was used to identify the product. MALDI-TOF-MS calculated for: CH-PEG₂-c(RGDfk) [C₉₃H₁₀₅N₁₇O₂₀S₂] (M.W.): 1,845.1. Found: 1,845.1.

### Example 23

Synthesis of CH-3c(RGDyk):

Dissolve 1 mg CH1055 (1.03 μmol) in 100 μl of dry DMSO. Then 0.48 mg NHS (4.12 μmol), 0.65 mg EDC (3.4 μmol) were added and the mixture reacted for 2 h at room temperature. Then 2.23 mg c(RGDyk) (3.605 μmol), 10.8 μl DIPEA (7.99 mg, 61.8 μmol) were added. The reaction solution was stirred overnight at room temperature under a nitrogen atmosphere. After the reaction finished, 100 μl of water was added and stirred for 1 h to quench the reaction. Finally, Dionex Summit high-performance liquid chromatography (HPLC) system (Dionex Corporation, Sunnyvale, Calif., USA), 340U four-channel ultraviolet-visible absorbance detector, Dionex C4, 9.4 mm×250 mm semi-preparative column, gradient elution starting from 5% acetonitrile and ending up with 95% acetonitrile (in water) at 35 min, 3 ml min⁻¹ flow rate, 254 nm and 650 nm detection wavelength was used to purify the reaction. Overall, 0.64 mg CH-3c(RGDyk) (yield 22.3%) was produced as a green solid. MALDI-TOF-MS was used to identify the product. MALDI-TOF-MS calculated for CH-3c(RGDyk) [C₁₃₅H₁₆₁N₃₃O₂₉S₂] (M.W.): 2,774.1. Found: 2,774.7.

### Example 24

Measuring NIR-II Quantum Yield:

The fluorescence quantum yield of CH-RGD NPs was measured in a similar manner as described in previous publications (Antaris et al., (2016) Nat. Mater. 15, 235-242; Antaris et al. (2017) Nat. Commun. 8: 15269). Briefly, IR-26 was utilized as a reference fluorophore (quantum yield of 0.05% in DCE). A serial dilution of five solutions of CH1055 derivatives as well as IR26 with an OD less than 0.1 at 808 nm was measured to confirm absorbance values at 808 nm and the fluorescent emission spectrum was collected on a wavelength-corrected NIR-II spectrometer in a 1 cm quartz cuvette (Starna) in the manner specified at mean paper methods. The fluorescent emission spectrum was integrated and plotted against the OD value at 808 nm and a linear fit was applied to verify the linearity between fluorescent brightness and concentration. For the brightest samples, inter-filter effects were seen approximately OD 0.1 at 808 nm, thus lower concentration ranges were utilized. By comparing the slope of the linear fit between IR26 and CH1055 derivatives, the quantum yield was determined based on the following supporting equation (1):

$${QY}_{sample} = {QY}_{ref} \times \frac{{slope}_{sample}}{{slope}_{ref}} \times {( \frac{{n}_{sample}}{{n}_{ref}} )}^{2} \qquad ( 1 )$$

where QYₛₐₘₚₗₑ is the QY of CH1055 derivatives, QY_{ref} is the QY of IR26 in DCE (0.05%), nₛₐₘₚₗₑ and n_{ref} are the refractive indices of IR26, CH1055 derivatives which are DCE (1.44) and water (1.33).

We claim:

1. A compound comprising a dye molecule attached to at least one cyclopeptide, wherein the composition at a first pH is a monomer and is a self-aggregate when at a second pH, wherein the second pH is less than the first pH.

2. The compound of claim 1, wherein the at least one cyclopeptide is conjugated directly to the dye molecule or is attached thereto via a linker.

3. The compound of claim 1, wherein the at least one cyclopeptide is according to Formula I 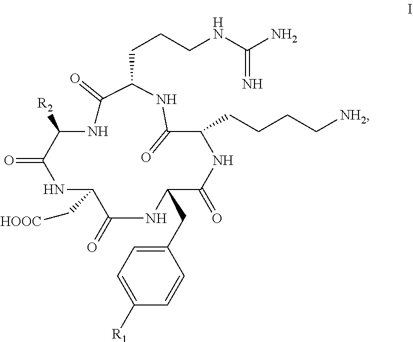
wherein R₁ is OH or H and R₂ is H or CH₃.

4. The compound of claim 1, wherein the dye molecule is a NIR-II dye.

5. The compound of claim 1, wherein the dye molecule has a formula selected from the group consisting of:

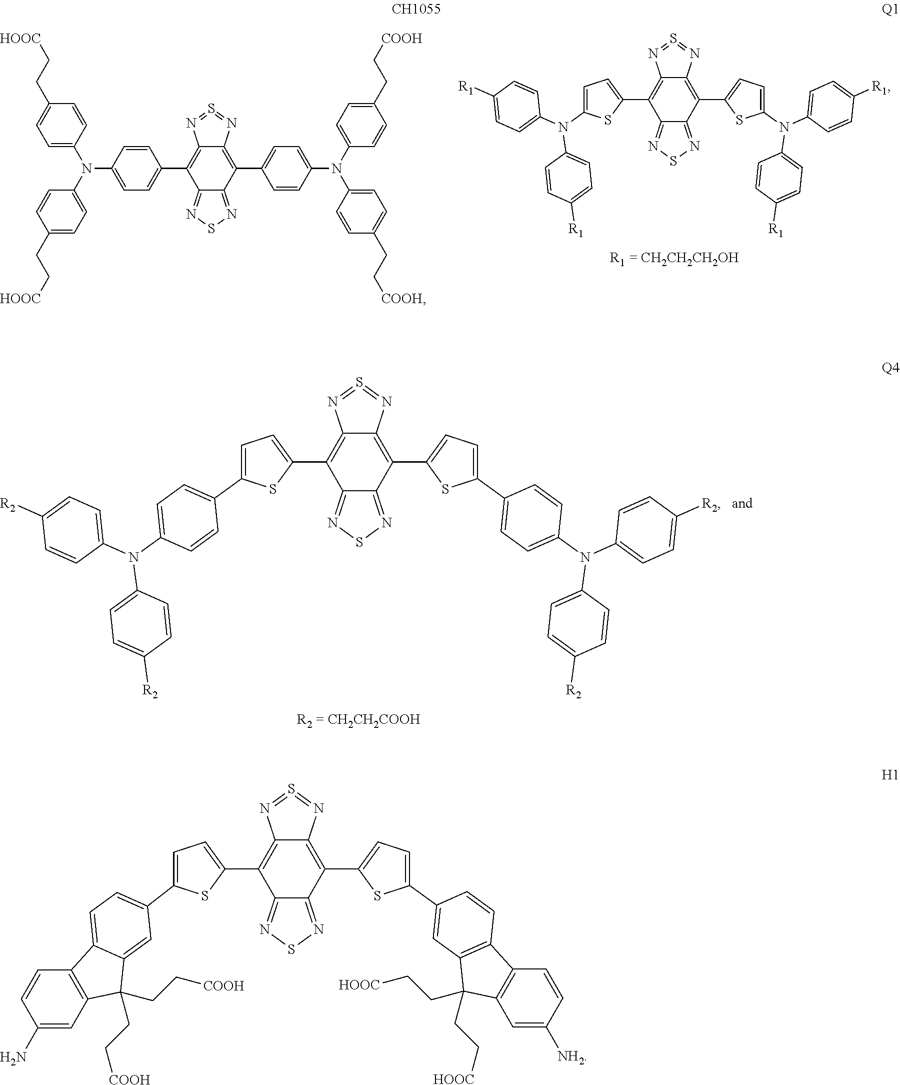

6. The compound of claim 1, wherein the cyclopeptide is attached to the dye molecule via a polyethylene glycol linker.

7. The compound of claim 1, wherein the compound forms a self-aggregate when at a pH ranging from about 6.5 to about 7.0.

8. The compound of claim 1, wherein the compound forms a self-aggregate when at a pH lower than about 6.5.

9. A composition comprising:
a compound comprising a dye molecule attached to at least one cyclopeptide, wherein the composition is a monomer at a first pH and forms a self-aggregate at a second pH, wherein the second pH is less than the first pH; and
a pharmaceutically acceptable carrier.

10. The composition of claim 9, wherein the at least one cyclopeptide is conjugated directly to the dye molecule or is attached thereto via a linker.

11. The composition of claim 9, wherein the at least one cyclopeptide is according to Formula I 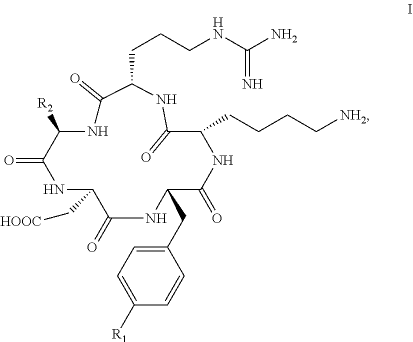
wherein R₁ is OH or H and R₂ is H or CH₃.

13. The composition of claim 9, wherein the dye molecule is a NIR-II dye.

14. The composition of claim 9, wherein the dye molecule has a formula selected from the group consisting of:

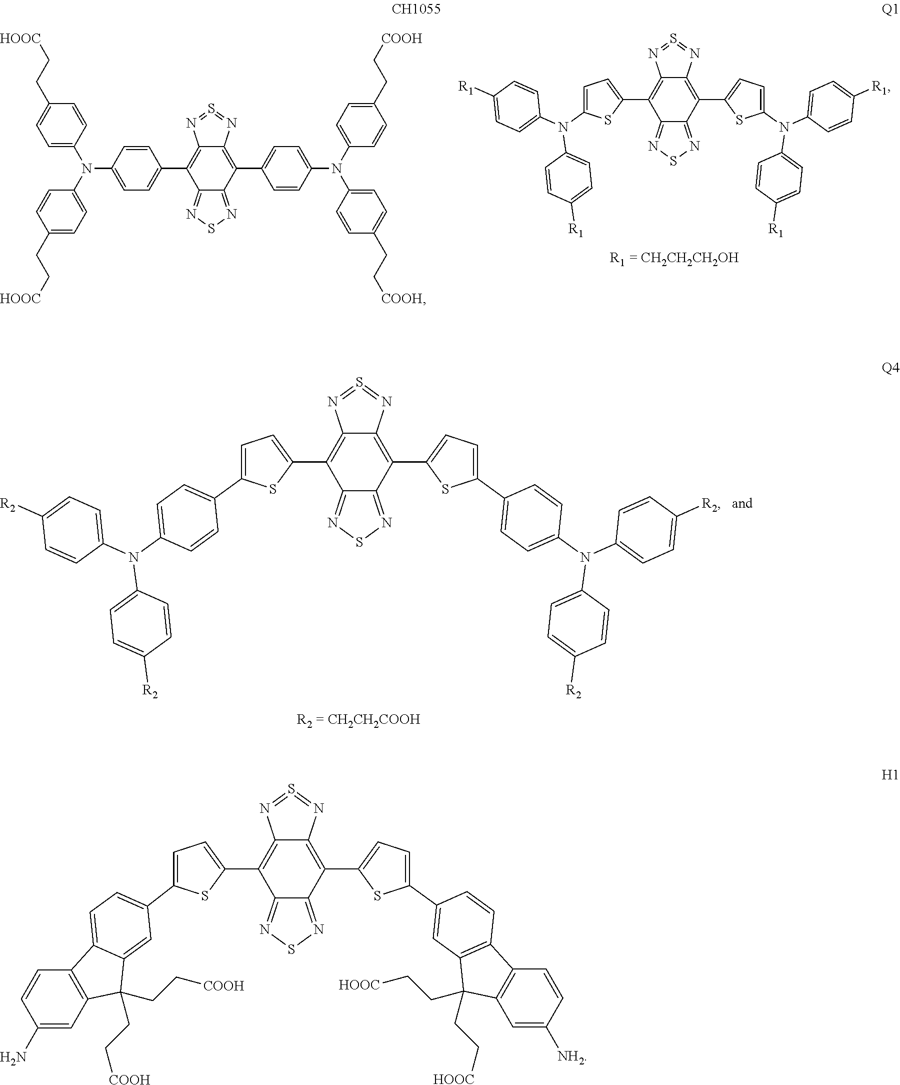

15. The composition of claim 9, wherein the at least one cyclopeptide is attached to the dye molecule via a polyethylene glycol linker.

16. The composition of claim 9, wherein the composition has a pH greater than the pH at which the compound forms a self-aggregate.

17. The composition of claim 9, wherein the composition has a pH at which the compound forms a self-aggregate.

18. A method comprising:
administering to an animal or human subject a composition comprising:
a compound comprising a dye molecule attached to at least one cyclopeptide, wherein the composition is a monomer at a first pH and forms a self-aggregate at a second pH, wherein the second pH is less than the first pH; and
a pharmaceutically acceptable carrier.

19. The method of claim 12, wherein the at least one cyclopeptide is according to Formula I 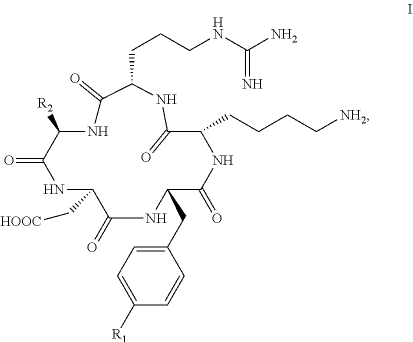
wherein R₁ is OH or H and R₂ is H or CH₃, and the dye molecule has a formula selected from the group consisting of:

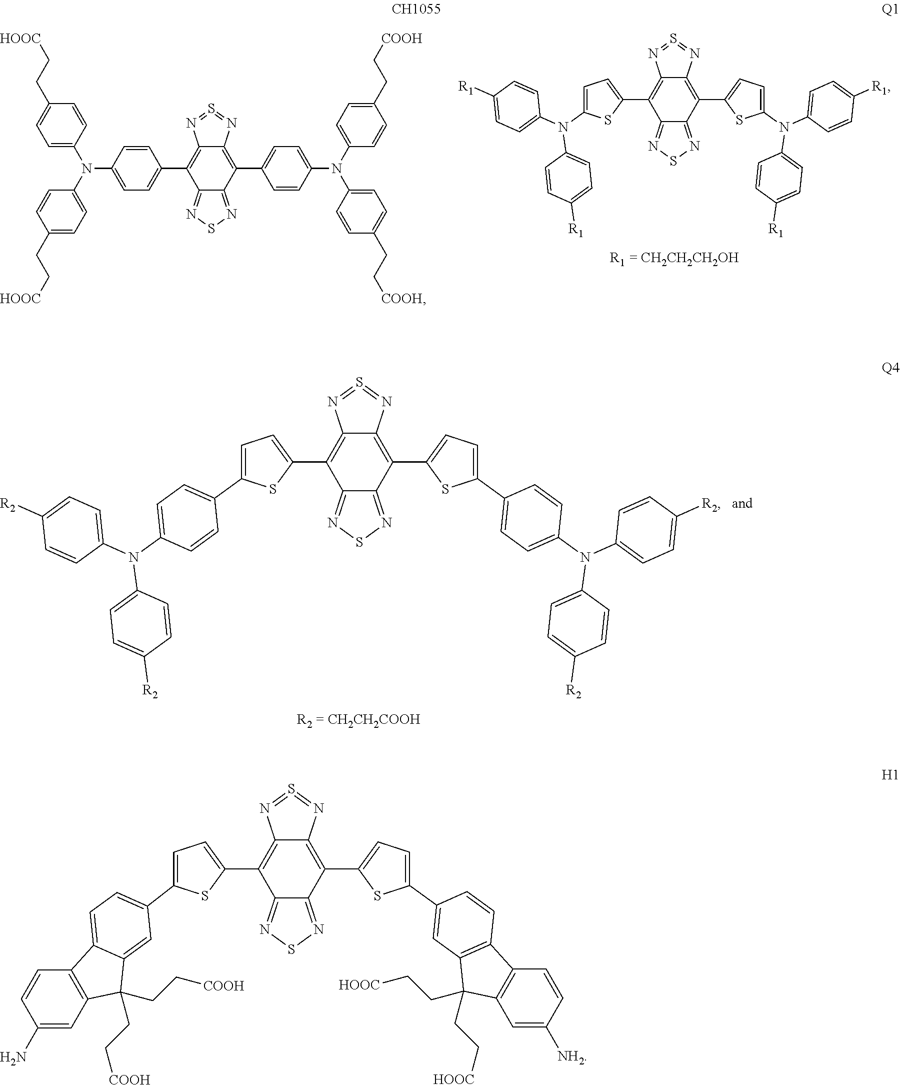

20. The method of claim 12, further comprising the step of imaging a portion of the subject using a fluorescent imaging technique.
